SEQUENCE LISTING (19 sequences)

SEQ ID NO: 1 - DNA, 13 bp, Artificial Sequence
  based on Homo Sapiens
nntggaaaannnn

SEQ ID NO: 2 - DNA, 11 bp, Artificial Sequence
  based on Homo Sapiens
tggaaannyny

SEQ ID NO: 3 - DNA, 13 bp, Artificial Sequence
  based on Homo Sapiens
ntggaaattttcn

SEQ ID NO: 4 - DNA, 2986 bp, Homo sapiens
agcaggaagctcgcgccgccgtcgccgccgccgctcagcttccccgggcgcgtccaggacccgctgcgccaggcgcgccgtccccggacccggcgtgcgtccctacgaggaaagggaccccgccgctcgagccgcctccgccagccccactgcgaggggtcccagagccagccgcgcccgccctcgcccccggccccgcagccttcccgccctgcgcgccatgaacgcccccgagcggcagccccaacccgacggcggggacgccccaggccacgagcctgggggcagcccccaagacgagcttgacttctccatcctcttcgactatgagtatttgaatccgaacgaagaagagccgaatgcacataaggtcgccagcccaccctccggacccgcataccccgatgatgtaatggactatggcctcaagccatacagcccccttgctagtctctctggcgagccccccggccgattcggagagccggatagggtagggccgcagaagtttctgagcgcggccaagccagcaggggcctcgggcctgagccctcggatcgagatcactccgtcccacgaactgatccaggcagtggggcccctccgcatgagagacgcgggcctcctggtggagcagcctcccctggccggggtggccgccagcccgaggttcaccctgcccgtgcccggcttcgagggctaccgcgagccgctttgcttgagccccgctagcagcggctcctctgccagcttcatttctgacaccttctccccctacacctcgccctgcgtctcgcccaataacggcgggcccgacgacctgtgtccgcagtttcaaaacatccctgctcattattcccccagaacctcgccaataatgtcacctcgaaccagcctcgccgaggacagctgcctgggccgccactcgcccgtgccccgtccggcctcccgctcctcatcgcctggtgccaagcggaggcattcgtgcgccgaggccttggttgccctgccgcccggagcctcaccccagcgctcccggagcccctcgccgcagccctcatctcacgtggcaccccaggaccacggctccccggctgggtacccccctgtggctggctctgccgtgatcatggatgccctgaacagcctcgccacggactcgccttgtgggatcccccccaagatgtggaagaccagccctgacccctcgccggtgtctgccgccccatccaaggccggcctgcctcgccacatctacccggccgtggagttcctggggccctgcgagcagggcgagaggagaaactcggctccagaatccatcctgctggttccgcccacttggcccaagccgctggtgcctgccattcccatctgcagcatcccagtgactgcatccctccctccacttgagtggccgctgtccagtcagtcaggctcttacgagctgcggatcgaggtgcagcccaagccacatcaccgggcccactatgagacagaaggcagccgaggggctgtcaaagctccaactggaggccaccctgtggttcagctccatggctacatggaaaacaagcctctgggacttcagatcttcattgggacagctgatgagcggatccttaagccgcacgccttctaccaggtgcaccgaatcacggggaaaactgtcaccaccaccagctatgagaagatagtgggcaacaccaaagtcctggagatccccttggagcccaaaaacaacatgagggcaaccatcgactgtgcggggatcttgaagcttagaaacgccgacattgagctgcggaaaggcgagacggacattggaagaaagaacacgcgggtgagactggttttccgagttcacatcccagagtccagtggcagaatcgtctctttacagactgcatctaaccccatcgagtgctcccagcgatctgctcacgagctgcccatggttgaaagacaagacacagacagctgcctggtctatggcggccagcaaatgatcctcacggggcagaactttacatccgagtccaaagttgtgtttactgagaagaccacagatggacagcaaatttgggagatggaagccacggtggataaggacaagagccagcccaacatgctttttgttgagatccctgaatatcggaacaagcatatccgcacacctgtaaaagtgaacttctacgtcatcaatgggaagagaaaacgaagtcagcctcagcactttacctaccacccagtcccagccatcaagacggagcccacggatgaatatgaccccactctgatctgcagccccacccatggaggcctggggagccagccttactacccccagcacccgatggtggccgagtccccctcctgcctcgtggccaccatggctccctgccagcagttccgcacggggctctcatcccctgacgcccgctaccagcaacagaacccagcggccgtactctaccagcggagcaagagcctgagccccagcctgctgggctatcagcagccggccctcatggccgccccgctgtcccttgcggacgctcaccgctctgtgctggtgcacgccggctcccagggccagagctcagccctgctccacccctctccgaccaaccagcaggcctcgcctgtgatccactactcacccaccaaccagcagctgcgctgcggaagccaccaggagttccagcacatcatgtactgcgagaatttcgcaccaggcaccaccagacctggcccgcccccggtcagtcaaggtcagaggctgagcccgggttcctaccccacagtcattcagcagcagaatgccacgagccaaagagccgccaaaaacggacccccggtcagtgaccaaaaggaagtattacctgcgggggtgaccattaaacaggagcagaacttggaccagacctacttggatgatgagctgatagacacacaccttagctggatacaaaacatattatga

SEQ ID NO: 5 - protein, 921 aa, Homo sapiens [sequence omitted]

SEQ ID NO: 6 - DNA, 3969 bp, Homo sapiens
cggctgcggttcctggtgctgctcggcgcgcggccagctttcggaacggaacgctcggcgtcgcgggccccgcccggaaagtttgccgtggagtcgcgacctcttggcccgcgcggcccggcatgaagcggcgttgaggagctgctgccgccgcttgccgctgccgccgccgccgcctgaggaggagctgcagcaccctgggccacgccgatgactactgcaaactgtggcgcccacgacgagctcgacttcaaactcgtctttggcgaggacggggcgccggcgccgccgcccccgggctcgcggcctgcagatcttgagccagatgattgtgcatccatttacatctttaatgtagatccacctccatctactttaaccacaccactttgcttaccacatcatggattaccgtctcactcttctgttttgtcaccatcgtttcagctccaaagtcacaaaaactatgaaggaacttgtgagattcctgaatctaaatatagcccattaggtggtcccaaaccctttgagtgcccaagtattcaaattacatctatctctcctaactgtcatcaagaattagatgcacatgaagatgacctacagataaatgacccagaacgggaatttttggaaaggccttctagagatcatctctatcttcctcttgagccatcctaccgggagtcttctcttagtcctagtcctgccagcagcatctcttctaggagttggttctctgatgcatcttcttgtgaatcgctttcacatatttatgatgatgtggactcagagttgaatgaagctgcagcccgatttacccttggatcccctctgacttctcctggtggctctccagggggctgccctggagaagaaacttggcatcaacagtatggacttggacactcattatcacccaggcaatctccttgccactctcctagatccagtgtcactgatgagaattggctgagccccaggccagcctcaggaccctcatcaaggcccacatccccctgtgggaaacggaggcactccagtgctgaagtttgttatgctgggtccctttcaccccatcactcacctgttccttcacctggtcactcccccaggggaagtgtgacagaagatacgtggctcaatgcttctgtccatggtgggtcaggccttggccctgcagtttttccatttcagtactgtgtagagactgacatccctctcaaaacaaggaaaacttctgaagatcaagctgccatactaccaggaaaattagagctgtgttcagatgaccaagggagtttatcaccagcccgggagacttcaatagatgatggccttggatctcagtatcctttaaagaaagattcatgtggtgatcagtttctttcagttccttcaccctttacctggagcaaaccaaagcctggccacacccctatatttcgcacatcttcattacctccactagactggcctttaccagctcattttggacaatgtgaactgaaaatagaagtgcaacctaaaactcatcatcgagcccattatgaaactgaaggtagccgaggggcagtaaaagcatctactgggggacatcctgttgtgaagctcctgggctataacgaaaagccaataaatctacaaatgtttattgggacagcagatgatcgatatttacgacctcatgcattttaccaggtgcatcgaatcactgggaagacagtcgctactgcaagccaagagataataattgccagtacaaaagttctggaaattccacttcttcctgaaaataatatgtcagccagtattgattgtgcaggtattttgaaactccgcaattcagatatagaacttcgaaaaggagaaactgatattggcagaaagaatactagagtacgacttgtgtttcgtgtacacatcccacagcccagtggaaaagtcctttctctgcagatagcctctatacccgttgagtgctcccagcggtctgctcaagaacttcctcatattgagaagtacagtatcaacagttgttctgtaaatggaggtcatgaaatggttgtgactggatctaattttcttccagaatccaaaatcatttttcttgaaaaaggacaagatggacgacctcagtgggaggtagaagggaagataatcagggaaaaatgtcaaggggctcacattgtccttgaagttcctccatatcataacccagcagttacagctgcagtgcaggtgcacttttatctttgcaatggcaagaggaaaaaaagccagtctcaacgttttacttatacaccagttttgatgaagcaagaacacagagaagagattgatttgtcttcagttccatctttgcctgtgcctcatcctgctcagacccagaggccttcctctgattcagggtgttcacatgacagtgtactgtcaggacagagaagtttgatttgctccatcccacaaacatatgcatccatggtgacctcatcccatctgccacagttgcagtgtagagatgagagtgttagtaaagaacagcatatgattccttctccaattgtacaccagccttttcaagtcacaccaacacctcctgtggggtcttcctatcagcctatgcaaactaatgttgtgtacaatggaccaacttgtcttcctattaatgctgcctctagtcaagaatttgattcagttttgtttcagcaggatgcaactctttctggtttagtgaatcttggctgtcaaccactgtcatccataccatttcattcttcaaattcaggctcaacaggacatctcttagcccatacacctcattctgtgcataccctgcctcatctgcaatcaatgggatatcattgttcaaatacaggacaaagatctctttcttctccagtggctgaccagattacaggtcagccttcgtctcagttacaacctattacatatggtccttcacattcagggtctgctacaacagcttccccagcagcttctcatcccttggctagttcaccgctttctgggccaccatctcctcagcttcagcctatgccttaccaatctcctagctcaggaactgcctcatcaccgtctccagccaccagaatgcattctggacagcactcaactcaagcacaaagtacgggccaggggggtctttctgcaccttcatccttaatatgtcacagtttgtgtgatccagcgtcatttccacctgatggggcaactgtgagcattaaacctgaaccagaagatcgagagcctaactttgcaaccattggtctgcaggacatcactttagatgatgaccaatttatatctgacttggaacaccagccatcaggttcagcagagaaatggcctaaccacagtgtgctctcatgtccagctcctttctggagaatctagaggtgaacgagataattgggagagacatgtcccagatttctgtttcccaaggagcaggggtgagcaggcaggctcccctcccgagtcctgagtccctggatttaggaagatctgatgggctctaacagtgcttactgcagccttgtgtccaccaccaacttctcagcatgtttctctccttggaccttgggtttccaactctgcagccttcaggtctggggccaggagtgggacccaccatttgtggggaaagtagcattcctccacctcaggccttgggtagatttggcaaaagaacaggagcagcataggctgtttgagctttggggaaatgaactttgctttttatatttaactaggatacttttatatgatgggtgctttgagtgtgaatgcagcaggctctcttgtttccgaggtgctgcttttgcaggtgacctggttacttagctaggattggtgatttgtactgctttatggtcatttgaagggccctttagtttttatgataatttttaaaataggaacttttgataagaccttctagaagcc

SEQ ID NO: 7 - protein, 1068 aa, Homo sapiens
MTTANCGAHDELDFKLVFGEDGAPAPPPPGSRPADLEPDDCASIYIFNVDPPPSTLTTPLCLPHHGLPSHSSVLSPSFQLQSHKNYEGTCEIPESKYSPLGGPKPFECPSIQITSISPNCHQELDAHEDDLQINDPEREFLERPSRDHLYLPLEPSYRESSLSPSPASSISSRSWFSDASSCESLSHIYDDVDSELNEAAARFTLGSPLTSPGGSPGGCPGEETWHQQYGLGHSLSPRQSPCHSPRSSVTDENWLSPRPASGPSSRPTSPCGKRRHSSAEVCYAGSLSPHHSPVPSPGHSPRGSVTEDTWLNASVHGGSGLGPAVFPFQYCVETDIPLKTRKTSEDQAAILPGKLELCSDDQGSLSPARETSIDDGLGSQYPLKKDSCGDQFLSVPSPFTWSKPKPGHTPIFRTSSLPPLDWPLPAHFGQCELKIEVQPKTHHRAHYETEGSRGAVKASTGGHPVVKLLGYNEKPINLQMFIGTADDRYLRPHAFYQVHRITGKTVATASQEIIIASTKVLEIPLLPENNMSASIDCAGILKLRNSDIELRKGETDIGRKNTRVRLVFRVHIPQPSGKVLSLQIASIPVECSQRSAQELPHIEKYSINSCSVNGGHEMVVTGSNFLPESKIIFLEKGQDGRPQWEVEGKIIREKCQGAHIVLEVPPYHNPAVTAAVQVHFYLCNGKRKKSQSQRFTYTPVLMKQEHREEIDLSSVPSLPVPHPAQTQRPSSDSGCSHDSVLSGQRSLICSIPQTYASMVTSSHLPQLQCRDESVSKEQHMIPSPIVHQPFQVTPTPPVGSSYQPMQTNVVYNGPTCLPINAASSQEFDSVLFQQDATLSGLVNLGCQPLSSIPFHSSNSGSTGHLLAHTPHSVHTLPHLQSMGYHCSNTGQRSLSSPVADQITGQPSSQLQPITYGPSHSGSATTASPAASHPLASSPLSGPPSPQLQPMPYQSPSSGTASSPSPATRMHSGQHSTQAQSTGQGGLSAPSSLICHSLCDPASFPPDGATVSIKPEPEDREPNFATIGLQDITLDDDQFISDLEHQPSGSAEKWPNHSVLSCPAPFWRI

SEQ ID NO: 8 - DNA, 4455 bp, Homo sapiens
atgagtcagacaagcggtggtgaggcaggctcgcctcctccagctgttgttgctgctgatgcttcttcagctccctcctcttcctccatgggcggtgcttgcagctcctttaccacctcttccagccctaccatttattctacctcagtcaccgacagcaaggctatgcaagtggagagctgctcctcagccgtgggggtaagtaacagaggggtaagtgaaaagcagttaaccagtaacacagttcagcagcatccatcaacaccgaagaggcacacagtcttgtacatctcaccaccacctgaggacttgctggataacagtcggatgtcctgccaggatgaggggtgtggattggaatctgagcagagctgcagtatgtggatggaggattccccctccaacttcagtaacatgagcaccagttcctacaatgataacactgaggtacctcgtaaatcacgaaaacgaaatccaaagcagaggccgggggtcaaacgacgagattgtgaagaatctaatatggatatatttgatgccgacagtgccaaagcacctcactatgtgctttctcagcttaccacggacaacaaaggcaactcaaaagcgggaaatggaacattggaaaaccaaaaaggaactggagtaaagaagagccctatgttgtgtggacaatatcctgttaaaagtgagggaaaggagctgaagatagttgtacaacctgagacacagcaccgagctcggtacctgactgagggcagccgtggctcagtgaaagatagaacacagcaaggctttcctacagtaaagctggaaggccataatgaacctgtagtgttgcaagtgtttgtgggcaacgactctggacgagtgaaaccacatggattttatcaggcctgcagagtaactggacgaaatacaactccttgcaaagaagtggacattgaaggcactactgttatagaagtcggccttgatcctagcaacaacatgacactggcggtggactgcgtagggatattgaaattgaggaatgctgatgtcgaagccagaataggaattgctggttccaagaagaaaagcactcgtgccagattggtttttcgagttaatatcatgaggaaagatggctccactttgacactgcaaacaccctcttctccaattttgtgtactcagccagcaggagtgccagaaatcttaaagaaaagcttgcatagctgttcagtgaaaggagaagaagaagtgtttttaatcggcaagaactttctgaaaggaactaaagttattttccaagaaaatgtttctgatgaaaactcttggaagtcagaagctgaaattgatatggaactatttcatcagaatcatcttattgtgaaggttcctccctatcatgaccaacatataactttgcctgtgtcagtgggaatatatgtagtgacaaatgctggaagatctcatgatgttcaaccattcacttacactccagacccagcagcagctggtgctttgaatgtaaatgtgaagaaggaaatatctagtccagcaagaccttgctcttttgaagaggccatgaaagcaatgaaaactactggatgtaatttagataaggtaaatattatccctaatgccctgatgactccactcataccaagcagtatgattaagagtgaagatgttactccaatggaagtaacagcagaaaaaagatcttccactatttttaagactacaaagtctgttggatcaactcagcaaacattagaaaacatctcaaacatagcaggaaatggctctttttcatcaccatcatcttcccacctaccttctgaaaatgaaaaacagcagcagattcagcccaaggcatacaacccagagaccctgacaactattcaaacccaggacatctcacagcctggtacttttccagcagtttctgcttctagtcagctgcccaacagcgatgcactattgcagcaggctacacagtttcagacaagagaaactcagtctagagagatattacagtcagatggtacagtggttaatttgtcacaactgactgaggcatcacaacaacagcagcagtcaccactacaagaacaagcacagactttacagcagcagatttcatcaaatatttttccatcaccaaatagtgtgagtcagcttcagaatactattcagcagctgcaagcagggagtttcacaggcagtactgctagtggcagcagtggaagtgttgacttggtccaacaagttttagaggcacagcagcagttatcttcagttttattttctgctccagatggtaatgagaatgttcaagagcagcttagtgcagatatttttcaacaagtcagtcaaattcagagtggtgtaagccctggaatgttttcctcaacagagccaacagtccataccagaccagataatttattacctggaagagctgaaagtgttcatccacagtctgaaaacacgttatctaatcaacagcagcagcagcagcagcaacagcaagtgatggaatcttcagccgcaatggtgatggagatgcaacagagtatctgccaggcagctgcccagattcagtcagagttattcccttcaactgcttcagcaaatggaaaccttcagcaatcgccagtttaccagcagacttctcacatgatgagtgcattgtctaccaatgaggatatgcaaatgcagtgtgaattgttttcttctcctcctgcagtttctggaaatgaaacttctacaactaccacacagcaggttgcaacccctggcactaccatgtttcagacatcaagttcaggagatggagaagaaactggaacacaagcaaaacagattcagaacagtgtctttcagaccatggtccaaatgcaacatagtggggacaatcaacctcaagttaaccttttttcatccacaaaaagtatgatgagtgttcagaatagtggtacccaacaacaaggtaatggtttattccagcaagggaatgagatgatgtcacttcaatctggaaattttttgcagcagtcttctcattcacaggcccaactttttcatcctcaaaatcctattgccgatgctcagaacctttcccaggaaactcaaggttctctctttcatagtccaaatcctattgtccacagtcagacttctacaacctcctctgaacaaatgcagcctccaatgtttcactctcaaagtaccattgctgtgttacagggctcttcagttcctcaagaccagcagtcaaccaacatatttctttcccagagtcccatgaataatcttcagactaacacagtagcccaagaagcattttttgcagcaccgaactcaatttctccacttcagtcaacatcaaacagtgaacaacaagctgctttccaacagcaagctccaatatcacacatccagactcctatgctttcccaagaacaggcacaacccccgcagcagggtttatttcagcctcaggtggccctgggctcccttccacctaatccaatgcctcaaagccaacaaggaaccatgttccagtcacagcactcaatagttgccatgcagagtaactctccatcccaggaacagcagcagcagcagcaacagcagcagcaacagcagcagcaacaacaacagagcattttattcagtaatcagaataccatggctacaatggcgtctccaaagcaaccaccaccaaacatgatattcaacccaaatcaaaatccaatggctaatcaggagcaacagaaccagtcaatttttcaccaacaaagtaacatggccccaatgaatcaagagcaacagcccatgcaatttcagagtcagtccacagtttcctcacttcagaacccaggtcctacccagtcggaatcatcacagacccccttgttccatagctctcctcagattcagttggtacaagggtcacctagttctcaagagcagcaagtaactctcttcttatctccagcatccatgtctgccttgcagaccagtataaatcaacaagatatgcaacagtctcctctttattcccctcagaacaacatgcctggaattcaaggagccacatcttcgcctcaaccacaggctactttatttcacaacacagcaggaggcacaatgaaccaactgcagaattctcctggctcatctcagcagacatcaggaatgttcttatttggcattcaaaataactgtagtcagcttttaacctctggaccagctacattgcctgatcagttgatggccataagtcagccaggccaaccacaaaacgagggccagccacctgtgacaacacttctttctcagcaaatgccagagaattctccactggcatcctctataaacaccaaccagaacatcgaaaagattgatttgcttgtttcattgcaaaaccaagggaacaacttgactggctccttttaa

SEQ ID NO: 9 - protein, 1484 aa, Homo sapiens
MSQTSGGEAGSPPPAVVAADASSAPSSSSMGGACSSFTTSSSPTIYSTSVTDSKAMQVESCSSAVGVSNRGVSEKQLTSNTVQQHPSTPKRHTVLYISPPPEDLLDNSRMSCQDEGCGLESEQSCSMWMEDSPSNFSNMSTSSYNDNTEVPRKSRKRNPKQRPGVKRRDCEESNMDIFDADSAKAPHYVLSQLTTDNKGNSKAGNGTLENQKGTGVKKSPMLCGQYPVKSEGKELKIVVQPETQHRARYLTEGSRGSVKDRTQQGFPTVKLEGHNEPVVLQVFVGNDSGRVKPHGFYQACRVTGRNTTPCKEVDIEGTTVIEVGLDPSNNMTLAVDCVGILKLRNADVEARIGIAGSKKKSTRARLVFRVNIMRKDGSTLTLQTPSSPILCTQPAGVPEILKKSLHSCSVKGEEEVFLIGKNFLKGTKVIFQENVSDENSWKSEAEIDMELFHQNHLIVKVPPYHDQHITLPVSVGIYVVTNAGRSHDVQPFTYTPDPAAAGALNVNVKKEISSPARPCSFEEAMKAMKTTGCNLDKVNIIPNALMTPLIPSSMIKSEDVTPMEVTAEKRSSTIFKTTKSVGSTQQTLENISNIAGNGSFSSPSSSHLPSENEKQQQIQPKAYNPETLTTIQTQDISQPGTFPAVSASSQLPNSDALLQQATQFQTRETQSREILQSDGTVVNLSQLTEASQQQQQSPLQEQAQTLQQQISSNIFPSPNSVSQLQNTIQQLQAGSFTGSTASGSSGSVDLVQQVLEAQQQLSSVLFSAPDGNENVQEQLSADIFQQVSQIQSGVSPGMFSSTEPTVHTRPDNLLPGRAESVHPQSENTLSNQQQQQQQQQQVMESSAAMVMEMQQSICQAAAQIQSELFPSTASANGNLQQSPVYQQTSHMMSALSTNEDMQMQCELFSSPPAVSGNETSTTTTQQVATPGTTMFQTSSSGDGEETGTQAKQIQNSVFQTMVQMQHSGDNQPQVNLFSSTKSMMSVQNSGTQQQGNGLFQQGNEMMSLQSGNFLQQSSHSQAQLFHPQNPIADAQNLSQETQGSLFHSPNPIVHSQTSTTSSEQMQPPMFHSQSTIAVLQGSSVPQDQQSTNIFLSQSPMNNLQTNTVAQEAFFAAPNSISPLQSTSNSEQQAAFQQQAPISHIQTPMLSQEQAQPPQQGLFQPQVALGSLPPNPMPQSQQGTMFQSQHSIVAMQSNSPSQEQQQQQQQQQQQQQQQQQSILFSNQNTMATMASPKQPPPNMIFNPNQNPMANQEQQNQSIFHQQSNMAPMNQEQQPMQFQSQSTVSSLQNPGPTQSESSQTPLFHSSPQIQLVQGSPSSQEQQVTLFLSPASMSALQTSINQQDMQQSPLYSPQNNMPGIQGATSSPQPQATLFHNTAGGTMNQLQNSPGSSQQTSGMFLFGIQNNCSQLLTSGPATLPDQLMAISQPGQPQNEGQPPVTTLLSQQMPENSPLASSINTNQNIEKIDLLVSLQNQGNNLTGSF

SEQ ID NO: 10 - DNA, 5575 bp, Homo sapiens
gcgcgacccgtcccgggggtggggccgggcgcagcggcgagaggaggcgaaggtggctgcggtagcagcagcgcggcagcctcggacccagcccggagcgcagggcggccgctgcaggtccccgctcccctccccgtgcgtccgcccatggccgccgccgggcagctgtgcttgctctacctgtcggcggggctcctgtcccggctcggcgcagccttcaacttggacactcgggaggacaacgtgatccggaaatatggagaccccgggagcctcttcggcttctcgctggccatgcactggcaactgcagcccgaggacaagcggctgttgctcgtgggggccccgcgggcagaagcgcttccactgcagagagccaacagaacgggagggctgtacagctgcgacatcaccgcccgggggccatgcacgcggatcgagtttgataacgatgctgaccccacgtcagaaagcaaggaagatcagtggatgggggtcaccgtccagagccaaggtccagggggcaaggtcgtgacatgtgctcaccgatatgaaaaaaggcagcatgttaatacgaagcaggaatcccgagacatctttgggcggtgttatgtcctgagtcagaatctcaggattgaagacgatatggatgggggagattggagcttttgtgatgggcgattgagaggccatgagaaatttggctcttgccagcaaggtgtagcagctacttttactaaagactttcattacattgtatttggagccccgggtacttataactggaaagggattgttcgtgtagagcaaaagaataacactttttttgacatgaacatctttgaagatgggccttatgaagttggtggagagactgagcatgatgaaagtctcgttcctgttcctgctaacagttacttaggtttttctttggactcagggaaaggtattgtttctaaagatgagatcacttttgtatctggtgctcccagagccaatcacagtggagccgtggttttgctgaagagagacatgaagtctgcacatctcctccctgagcacatattcgatggagaaggtctggcctcttcatttggctatgatgtggcggtggtggacctcaacaaggatgggtggcaagatatagttattggagccccacagtattttgatagagatggagaagttggaggtgcagtgtatgtctacatgaaccagcaaggcagatggaataatgtgaagccaattcgtcttaatggaaccaaagattctatgtttggcattgcagtaaaaaatattggagatattaatcaagatggctacccagatattgcagttggagctccgtatgatgacttgggaaaggtttttatctatcatggatctgcaaatggaataaataccaaaccaacacaggttctcaagggtatatcaccttattttggatattcaattgctggaaacatggaccttgatcgaaattcctaccctgatgttgctgttggttccctctcagattcagtaactattttcagatcccggcctgtgattaatattcagaaaaccatcacagtaactcctaacagaattgacctccgccagaaaacagcgtgtggggcgcctagtgggatatgcctccaggttaaatcctgttttgaatatactgctaaccccgctggttataatccttcaatatcaattgtgggcacacttgaagctgaaaaagaaagaagaaaatctgggctatcctcaagagttcagtttcgaaaccaaggttctgagcccaaatatactcaagaactaactctgaagaggcagaaacagaaagtgtgcatggaggaaaccctgtggctacaggataatatcagagataaactgcgtcccattcccataactgcctcagtggagatccaagagccaagctctcgtaggcgagtgaattcacttccagaagttcttccaattctgaattcagatgaacccaagacagctcatattgatgttcacttcttaaaagagggatgtggagacgacaatgtatgtaacagcaaccttaaactagaatataaattttgcacccgagaaggaaatcaagacaaattttcttatttaccaattcaaaaaggtgtaccagaactagttctaaaagatcagaaggatattgctttagaaataacagtgacaaacagcccttccaacccaaggaatcccacaaaagatggcgatgacgcccatgaggctaaactgattgcaacgtttccagacactttaacctattctgcatatagagaactgagggctttccctgagaaacagttgagttgtgttgccaaccagaatggctcgcaagctgactgtgagctcggaaatccttttaaaagaaattcaaatgtcactttttatttggttttaagtacaactgaagtcacctttgacaccccagatctggatattaatctgaagttagaaacaacaagcaatcaagataatttggctccaattacagctaaagcaaaagtggttattgaactgcttttatcggtctcgggagttgctaaaccttcccaggtgtattttggaggtacagttgttggcgagcaagctatgaaatctgaagatgaagtgggaagtttaatagagtatgaattcagggtaataaacttaggtaaacctcttacaaacctcggcacagcaaccttgaacattcagtggccaaaagaaattagcaatgggaaatggttgctttatttggtgaaagtagaatccaaaggattggaaaaggtaacttgtgagccacaaaaggagataaactccctgaacctaacggagtctcacaactcaagaaagaaacgggaaattactgaaaaacagatagatgataacagaaaattttctttatttgctgaaagaaaataccagactcttaactgtagcgtgaacgtgaactgtgtgaacatcagatgcccgctgcgggggctggacagcaaggcgtctcttattttgcgctcgaggttatggaacagcacatttctagaggaatattccaaactgaactacttggacattctcatgcgagccttcattgatgtgactgctgctgccgaaaatatcaggctgccaaatgcaggcactcaggttcgagtgactgtgtttccctcaaagactgtagctcagtattcgggagtaccttggtggatcatcctagtggctattctcgctgggatcttgatgcttgctttattagtgtttatactatggaagtgtggtttcttcaagagaaataagaaagatcattatgatgccacatatcacaaggctgagatccatgctcagccatctgataaagagaggcttacttctgatgcatagtattgatctacttctgtaattgtgtggattctttaaacgctctaggtacgatgacagtgttccccgataccatgctgtaaggatccggaaagaagagcgagagatcaaagatgaaaagtatattgataaccttgaaaaaaaacagtggatcacaaagtggaacgaaaatgaaagctactcatagcgggggcctaaaaaaaaaaagcttcacagtacccaaactgctttttccaactcagaaattcaatttggatttaaaagcctgctcaatccctgaggactgatttcagagtgactacacacagtacgaacctacagttttaactgtggatattgttacgtagcctaaggctcctgttttgcacagccaaatttaaaactgttggaatggatttttctttaactgccgtaatttaactttctgggttgcctttatttttggcgtggctgacttacatcatgtgttggggaagggcctgcccagttgcactcaggtgacatcctccagatagtgtagctgaggaggcacctacactcacctgcactaacagagtggccgtcctaacctcgggcctgctgcgcagacgtccatcacgttagctgtcccacatcacaagactatgccattggggtagttgtgtttcaacggaaagtgctgtcttaaactaaatgtgcaatagaaggtgatgttgccatcctaccgtcttttcctgtttcctagctgtgtgaatacctgctcacgtcaaatgcatacaagtttcattctccctttcactaaaacacacaggtgcaacagacttgaatgctagttatacttatttgtatatggtatttattttttcttttctttacaaaccattttgttattgactaacaggccaaagagtctccagtttacccttcaggttggtttaatcaatcagaattagagcatgggaggtcatcactttgacctaaattatttactgcaaaaagaaaatctttataaatgtaccagagagagttgttttaataacttatctataaactataacctctccttcatgacagcctccaccccacaacccaaaaggtttaagaaatagaattataactgtaaagatgtttatttcaggcattggatattttttactttagaagcctgcataatgtttctggatttcatactgtaacattcaggaattcttggagaaaatgggtttattcactgaactctagtgcggtttactcactgctgcaaatactgtatattcaggacttgaaagaaatggtgaatgcctatggtggatccaaactgatccagtataagactactgaatctgctaccaaaacagttaatcagtgagtcgatgttctattttttgttttgtttcctcccctatctgtattcccaaaaattactttggggctaatttaacaagaactttaaattgtgttttaattgtaaaaatggcagggggtggaattattactctatacattcaacagagactgaatagatatgaaagctgattttttttaattaccatgcttcacaatgttaagttatatggggagcaacagcaaacaggtgctaatttgttttggatatagtataagcagtgtctgtgttttgaaagaatagaacacagtttgtagtgccactgttgttttgggggggcttttttcttttcggaaatcttaaaccttaagatactaaggacgttgttttggttgtactttggaattcttagtcacaaaatatattttgtttacaaaaatttctgtaaaacaggttataacagtgtttaaagtctcagtttcttgcttggggaacttgtgtccctaatgtgtttagattgctagattgctaaggagctgatactttgacagtgtttttagacctgtgttactaaaaaaaagatgaatgtcctgaaaagggtgttgggagggtggttcaacaaagaaacaaagatgttatggtgtttagatttatggttgttaaaaatgtcatctcaagtcaagtcactggtctgtttgcatttgatacatttttgtactaactagcattgtaaaattatttcatgattagaaattacctgtggatatttgtataaaagtgtgaaataaattttttataaaagtgttcattgtttcgtaacacagcattgtatatgtgaagcaaactctaaaattataaatgacaacctgaattatctatttcatc

SEQ ID NO: 11 - protein, 1073 aa, Homo sapiens
MAAAGQLCLLYLSAGLLSRLGAAFNLDTREDNVIRKYGDPGSLFGFSLAMHWQLQPEDKRLLLVGAPRAEALPLQRANRTGGLYSCDITARGPCTRIEFDNDADPTSESKEDQWMGVTVQSQGPGGKVVTCAHRYEKRQHVNTKQESRDIFGRCYVLSQNLRIEDDMDGGDWSFCDGRLRGHEKFGSCQQGVAATFTKDFHYIVFGAPGTYNWKGIVRVEQKNNTFFDMNIFEDGPYEVGGETEHDESLVPVPANSYLGFSLDSGKGIVSKDEITFVSGAPRANHSGAVVLLKRDMKSAHLLPEHIFDGEGLASSFGYDVAVVDLNKDGWQDIVIGAPQYFDRDGEVGGAVYVYMNQQGRWNNVKPIRLNGTKDSMFGIAVKNIGDINQDGYPDIAVGAPYDDLGKVFIYHGSANGINTKPTQVLKGISPYFGYSIAGNMDLDRNSYPDVAVGSLSDSVTIFRSRPVINIQKTITVTPNRIDLRQKTACGAPSGICLQVKSCFEYTANPAGYNPSISIVGTLEAEKERRKSGLSSRVQFRNQGSEPKYTQELTLKRQKQKVCMEETLWLQDNIRDKLRPIPITASVEIQEPSSRRRVNSLPEVLPILNSDEPKTAHIDVHFLKEGCGDDNVCNSNLKLEYKFCTREGNQDKFSYLPIQKGVPELVLKDQKDIALEITVTNSPSNPRNPTKDGDDAHEAKLIATFPDTLTYSAYRELRAFPEKQLSCVANQNGSQADCELGNPFKRNSNVTFYLVLSTTEVTFDTPDLDINLKLETTSNQDNLAPITAKAKVVIELLLSVSGVAKPSQVYFGGTVVGEQAMKSEDEVGSLIEYEFRVINLGKPLTNLGTATLNIQWPKEISNGKWLLYLVKVESKGLEKVTCEPQKEINSLNLTESHNSRKKREITEKQIDDNRKFSLFAERKYQTLNCSVNVNCVNIRCPLRGLDSKASLILRSRLWNSTFLEEYSKLNYLDILMRAFIDVTAAAENIRLPNAGTQVRVTVFPSKTVAQYSGVPWWIILVAILAGILMLALLVFILWKCGFFKRNKKDHYDATYHKAEIHAQPSDKERLTSDA

SEQ ID NO: 12 - DNA, 5642 bp, Homo sapiens
cgcccgcgcgctgcagccccatctcctagcggcagcccaggcgcggagggagcgagtccgccccgaggtaggtccaggacgggcgcacagcagcagccgaggctggccgggagagggaggaagaggatggcagggccacgccccagcccatgggccaggctgctcctggcagccttgatcagcgtcagcctctctgggaccttggcaaaccgctgcaagaaggccccagtgaagagctgcacggagtgtgtccgtgtggataaggactgcgcctactgcacagacgagatgttcagggaccggcgctgcaacacccaggcggagctgctggccgcgggctgccagcgggagagcatcgtggtcatggagagcagcttccaaatcacagaggagacccagattgacaccaccctgcggcgcagccagatgtccccccaaggcctgcgggtccgtctgcggcccggtgaggagcggcattttgagctggaggtgtttgagccactggagagccccgtggacctgtacatcctcatggacttctccaactccatgtccgatgatctggacaacctcaagaagatggggcagaacctggctcgggtcctgagccagctcaccagcgactacactattggatttggcaagtttgtggacaaagtcagcgtcccgcagacggacatgaggcctgagaagctgaaggagccctggcccaacagtgacccccccttctccttcaagaacgtcatcagcctgacagaagatgtggatgagttccggaataaactgcagggagagcggatctcaggcaacctggatgctcctgagggcggcttcgatgccatcctgcagacagctgtgtgcacgagggacattggctggcgcccggacagcacccacctgctggtcttctccaccgagtcagccttccactatgaggctgatggcgccaacgtgctggctggcatcatgagccgcaacgatgaacggtgccacctggacaccacgggcacctacacccagtacaggacacaggactacccgtcggtgcccaccctggtgcgcctgctcgccaagcacaacatcatccccatctttgctgtcaccaactactcctatagctactacgagaagcttcacacctatttccctgtctcctcactgggggtgctgcaggaggactcgtccaacatcgtggagctgctggaggaggccttcaatcggatccgctccaacctggacatccgggccctagacagcccccgaggccttcggacagaggtcacctccaagatgttccagaagacgaggactgggtcctttcacatccggcggggggaagtgggtatataccaggtgcagctgcgggcccttgagcacgtggatgggacgcacgtgtgccagctgccggaggaccagaagggcaacatccatctgaaaccttccttctccgacggcctcaagatggacgcgggcatcatctgtgatgtgtgcacctgcgagctgcaaaaagaggtgcggtcagctcgctgcagcttcaacggagacttcgtgtgcggacagtgtgtgtgcagcgagggctggagtggccagacctgcaactgctccaccggctctctgagtgacattcagccctgcctgcgggagggcgaggacaagccgtgctccggccgtggggagtgccagtgcgggcactgtgtgtgctacggcgaaggccgctacgagggtcagttctgcgagtatgacaacttccagtgtccccgcacttccgggttcctctgcaatgaccgaggacgctgctccatgggccagtgtgtgtgtgagcctggttggacaggcccaagctgtgactgtcccctcagcaatgccacctgcatcgacagcaatgggggcatctgtaatggacgtggccactgtgagtgtggccgctgccactgccaccagcagtcgctctacacggacaccatctgcgagatcaactactcggcgatccacccgggcctctgcgaggacctacgctcctgcgtgcagtgccaggcgtggggcaccggcgagaagaaggggcgcacgtgtgaggaatgcaacttcaaggtcaagatggtggacgagcttaagagagccgaggaggtggtggtgcgctgctccttccgggacgaggatgacgactgcacctacagctacaccatggaaggtgacggcgcccctgggcccaacagcactgtcctggtgcacaagaagaaggactgccctccgggctccttctggtggctcatccccctgctcctcctcctcctgccgctcctggccctgctactgctgctatgctggaagtactgtgcctgctgcaaggcctgcctggcacttctcccgtgctgcaaccgaggtcacatggtgggctttaaggaagaccactacatgctgcgggagaacctgatggcctctgaccacttggacacgcccatgctgcgcagcgggaacctcaagggccgtgacgtggtccgctggaaggtcaccaacaacatgcagcggcctggctttgccactcatgccgccagcatcaaccccacagagctggtgccctacgggctgtccttgcgcctggcccgcctttgcaccgagaacctgctgaagcctgacactcgggagtgcgcccagctgcgccaggaggtggaggagaacctgaacgaggtctacaggcagatctccggtgtacacaagctccagcagaccaagttccggcagcagcccaatgccgggaaaaagcaagaccacaccattgtggacacagtgctgatggcgccccgctcggccaagccggccctgctgaagcttacagagaagcaggtggaacagagggccttccacgacctcaaggtggcccccggctactacaccctcactgcagaccaggacgcccggggcatggtggagttccaggagggcgtggagctggtggacgtacgggtgcccctctttatccggcctgaggatgacgacgagaagcagctgctggtggaggccatcgacgtgcccgcaggcactgccaccctcggccgccgcctggtaaacatcaccatcatcaaggagcaagccagagacgtggtgtcctttgagcagcctgagttctcggtcagccgcggggaccaggtggcccgcatccctgtcatccggcgtgtcctggacggcgggaagtcccaggtctcctaccgcacacaggatggcaccgcgcagggcaaccgggactacatccccgtggagggtgagctgctgttccagcctggggaggcctggaaagagctgcaggtgaagctcctggagctgcaagaagttgactccctcctgcggggccgccaggtccgccgtttccacgtccagctcagcaaccctaagtttggggcccacctggccagccccactccaccaccatcatcatcagggacccagatgaactggaccggagcttcacgagtcagatgttgtcatcacagccaccccctcacggcgacctgggcgccccgcagaaccccaatgctaaggccgctgggtccaggaagatccatttcaactggctgcccccttctggcaagccaatggggtacagggtaaagtactggattcagggtgactccgaatccgaagcccacctgctcgacagcaaggtgccctcagtggagctcaccaacctgtacccgtattgcgactatgagatgaaggtgtgcgcctacggggctcagggcgagggaccctacagctccctggtgtcctgccgcacccaccaggaagtgcccagcgagccagggcgtctggccttcaatgtcgtctcctccacggtgacccagctgagctgggctgagccggctgagaccaacggtgagatcacagcctacgaggtctgctatggcctggtcaacgatgacaaccgacctattgggcccatgaagaaagtgctggttgacaaccctaagaaccggatgctgcttattgagaaccttcgggagtcccagccctaccgctacacggtgaaggcgcgcaacggggccggctgggggcctgagcgggaggccatcatcaacctggccacccagcccaagaggcccatgtccatccccatcatccctgacatccctatcgtggacgcccagagcggggaggactacgacagcttccttatgtacagcgatgacgttctacgctctccatcgggcagccagaggcccagcgtctccgatgacactgagcacctggtgaatggccggatggactttgccttcccgggcagcaccaactccctgcacaggatgaccacgaccagtgctgctgcctatggcacccacctgagcccacacgtgccccaccgcgtgctaagcacatcctccaccctcacacgggactacaactcactgacccgctcagaacactcacactcgaccacactgcccagggactactccaccctcacctccgtctcctcccacgctctcgcctgactgctggtgtgcccgacacgcccacccgcctggtgttctctgccctggggcccacatctctcagagtgagctggcaggagccgcggtgcgagcggccgctgcagggctacagtgtggagtaccagctgctgaacggcggtgagctgcatcggctcaacatccccaaccctgcccagacctcggtggtggtggaagacctcctgcccaaccactcctacgtgttccgcgtgcgggcccagagccaggaaggctggggccgagagcgtgagggtgtcatcaccattgaatcccaggtgcacccgcagagcccactgtgtcccctgccaggctccgccttcactttgagcactcccagtgccccaggcccgctggtgttcactgccctgagcccagactcgctgcagctgagctgggagcggccacggaggcccaatggggatatcgtcggctacctggtgacctgtgagatggcccaaggaggagggccagccaccgcattccgggtggatggagacagccccgagagccggctgaccgtgccgggcctcagcgagaacgtgccctacaagttcaaggtgcaggccaggaccactgagggcttcgggccagagcgcgagggcatcatcaccatagagtcccaggatggaggacccttcccgcagctgggcagccgtgccgggctcttccagcacccgctgcaaagcgagtacagcagcatcaccaccacccacaccagcgccaccgagcccttcctagtggatgggctgaccctgggggcccagcacctggaggcaggcggctccctcacccggcatgtgacccaggagtttgtgagccggacactgaccaccagcggaacccttagcacccacatggaccaacagttcttccaaacttgaccgcaccctgccccacccccgccacgtcccactaggcgtcctcccgactcctctcccggagcctcctcagctactccatccttgcacccctggggcccagcccacccgcatgcacagagcaggggctaggtgtctcctgggaggcatgaagggggcaaggtccgtcctctgtgggcccaaacctatttgtaaccaaagagctgggagcagcacaaggacccagcctttgttctgcacttaataaatggttttgctactg

SEQ ID NO: 13 - protein, 1158 aa, Homo sapiens
MAGPRPSPWARLLLAALISVSLSGTLANRCKKAPVKSCTECVRVDKDCAYCTDEMFRDRRCNTQAELLAAGCQRESIVVMESSFQITEETQIDTTLRRSQMSPQGLRVRLRPGEERHFELEVFEPLESPVDLYILMDFSNSMSDDLDNLKKMGQNLARVLSQLTSDYTIGFGKFVDKVSVPQTDMRPEKLKEPWPNSDPPFSFKNVISLTEDVDEFRNKLQGERISGNLDAPEGGFDAILQTAVCTRDIGWRPDSTHLLVFSTESAFHYEADGANVLAGIMSRNDERCHLDTTGTYTQYRTQDYPSVPTLVRLLAKHNIIPIFAVTNYSYSYYEKLHTYFPVSSLGVLQEDSSNIVELLEEAFNRIRSNLDIRALDSPRGLRTEVTSKMFQKTRTGSFHIRRGEVGIYQVQLRALEHVDGTHVCQLPEDQKGNIHLKPSFSDGLKMDAGIICDVCTCELQKEVRSARCSFNGDFVCGQCVCSEGWSGQTCNCSTGSLSDIQPCLREGEDKPCSGRGECQCGHCVCYGEGRYEGQFCEYDNFQCPRTSGFLCNDRGRCSMGQCVCEPGWTGPSCDCPLSNATCIDSNGGICNGRGHCECGRCHCHQQSLYTDTICEINYSAIHPGLCEDLRSCVQCQAWGTGEKKGRTCEECNFKVKMVDELKRAEEVVVRCSFRDEDDDCTYSYTMEGDGAPGPNSTVLVHKKKDCPPGSFWWLIPLLLLLLPLLALLLLLCWKYCACCKACLALLPCCNRGHMVGFKEDHYMLRENLMASDHLDTPMLRSGNLKGRDVVRWKVTNNMQRPGFATHAASINPTELVPYGLSLRLARLCTENLLKPDTRECAQLRQEVEENLNEVYRQISGVHKLQQTKFRQQPNAGKKQDHTIVDTVLMAPRSAKPALLKLTEKQVEQRAFHDLKVAPGYYTLTADQDARGMVEFQEGVELVDVRVPLFIRPEDDDEKQLLVEAIDVPAGTATLGRRLVNITIIKEQARDVVSFEQPEFSVSRGDQVARIPVIRRVLDGGKSQVSYRTQDGTAQGNRDYIPVEGELLFQPGEAWKELQVKLLELQEVDSLLRGRQVRRFHVQLSNPKFGAHLASPTPPPSSSGTQMNWTGASRVRCCHHSHPLTATWAPRRTPMLRPLGPGRSISTGCPLLASQWGTG

SEQ ID NO: 14 - protein, 297 aa, Homo sapiens
GNGTLENQKGTGVKKSPMLCGQYPVKSEGKELKIVVQPETQHRARYLTEGSRGSVKDRTQQGFPTVKLEGHNEPVVLQVFVGNDSGRVKPHGFYQACRVTGRNTTPCKEVDIEGTTVIEVGLDPSNNMTLAVDCVGILKLRNADVEARIGIAGSKKKSTRARLVFRVNIMRKDGSTLTLQTPSSPILCTQPAGVPEILKKSLHSCSVKGEEEVFLIGKNFLKGTKVIFQENVSDENSWKSEAEIDMELFHQNHLIVKVPPYHDQHITLPVSVGIYVVTNAGRSHDVQPFTYTPDPAA

SEQ ID NO: 15 - protein, 1455 aa, Homo sapiens
MGGACSSFTTSSSPTIYSTSVTDSKAMQVESCSSAVGVSNRGVSEKQLTSNTVQQHPSTPKRHTVLYISPPPEDLLDNSRMSCQDEGCGLESEQSCSMWMEDSPSNFSNMSTSSYNDNTEVPRKSRKRNPKQRPGVKRRDCEESNMDIFDADSAKAPHYVLSQLTTDNKGNSKAGNGTLENQKGTGVKKSPMLCGQYPVKSEGKELKIVVQPETQHRARYLTEGSRGSVKDRTQQGFPTVKLEGHNEPVVLQVFVGNDSGRVKPHGFYQACRVTGRNTTPCKEVDIEGTTVIEVGLDPSNNMTLAVDCVGILKLRNADVEARIGIAGSKKKSTRARLVFRVNIMRKDGSTLTLQTPSSPILCTQPAGVPEILKKSLHSCSVKGEEEVFLIGKNFLKGTKVIFQENVSDENSWKSEAEIDMELFHQNHLIVKVPPYHDQHITLPVSVGIYVVTNAGRSHDVQPFTYTPDPAAAGALNVNVKKEISSPARPCSFEEAMKAMKTTGCNLDKVNIIPNALMTPLIPSSMIKSEDVTPMEVTAEKRSSTIFKTTKSVGSTQQTLENISNIAGNGSFSSPSSSHLPSENEKQQQIQPKAYNPETLTTIQTQDISQPGTFPAVSASSQLPNSDALLQQATQFQTRETQSREILQSDGTVVNLSQLTEASQQQQQSPLQEQAQTLQQQISSNIFPSPNSVSQLQNTIQQLQAGSFTGSTASGSSGSVDLVQQVLEAQQQLSSVLFSAPDGNENVQEQLSADIFQQVSQIQSGVSPGMFSSTEPTVHTRPDNLLPGRAESVHPQSENTLSNQQQQQQQQQQVMESSAAMVMEMQQSICQAAAQIQSELFPSTASANGNLQQSPVYQQTSHMMSALSTNEDMQMQCELFSSPPAVSGNETSTTTTQQVATPGTTMFQTSSSGDGEETGTQAKQIQNSVFQTMVQMQHSGDNQPQVNLFSSTKSMMSVQNSGTQQQGNGLFQQGNEMMSLQSGNFLQQSSHSQAQLFHPQNPIADAQNLSQETQGSLFHSPNPIVHSQTSTTSSEQMQPPMFHSQSTIAVLQGSSVPQDQQSTNIFLSQSPMNNLQTNTVAQEAFFAAPNSISPLQSTSNSEQQAAFQQQAPISHIQTPMLSQEQAQPPQQGLFQPQVALGSLPPNPMPQSQQGTMFQSQHSIVAMQSNSPSQEQQQQQQQQQQQQQQQQQSILFSNQNTMATMASPKQPPPNMIFNPNQNPMANQEQQNQSIFHQQSNMAPMNQEQQPMQFQSQSTVSSLQNPGPTQSESSQTPLFHSSPQIQLVQGSPSSQEQQVTLFLSPASMSALQTSINQQDMQQSPLYSPQNNMPGIQGATSSPQPQATLFHNTAGGTMNQLQNSPGSSQQTSGMFLFGIQNNCSQLLTSGPATLPDQLMAISQPGQPQNEGQPPVTTLLSQQMPENSPLASSINTNQNIEKIDLLVSLQNQGNNLTGSF

SEQ ID NO: 16 - DNA, 14239 bp, Homo sapiens
tgcaacggaaacttttggctccacgaacagaaaagcaagagcaaaaactcagttaaatgcgttcttcctccatctttgcctagtccaaaaggaaaaaaaaagaaacagaaaaaagaaaaactgtttggaagagtagcgttgaggtttgctttttacctgtttttcctctagaacagccaggtggattcacatgatccaattttttaattgtcttctatgtcccactccgaataacccggatgccccagcacaatcagagagatcgttttttttaaaaaaaatttcagcctccaaaggtaggaggggagtgttaggggagaaagtactttaattaaaaatcaataactcgaggtttttgggatacggtttgccatttcctaattaagaaatgggtttgacagtccctttgtacacactgctatgcaaaccccaaagggttggcggctgtccgggcgatgacactccggtcccctgcgagaccccgggccagccaggcccgtccgccgccggcctctggggtccgtccccggctcgcgcagacctctcgcttctctcggctctgtctcctgcgctcagctctgctcggggccggccgcctcaggctcgccgccaccaggtcgttgcaaataccttttccctccccggggccccagcgcgcggccacctcccagcctccccccctcccaccctggcagcggggccctttcccggctcaggaacagcagcagcccgggccgcgccggcaggaagcgaggcccatgttgctgctgttccctggcgcgcctccccgccctccgggggccgccacggctcttccgcgctcccgggcacccccctccgcgcctgcgctgtgccccacgggggcggggctcagattcctgtcagcggcggcggcggtggcggcgaccgtcagttttcgctgaggagaaacacgaaacggaccctttggctctcccccttccccttccccgtcctgaacccctctcctggtcaccgagaatcagtccccgtggagttccccctccacctcgccatcgtttcctcggtcctcggcccagtggaagtcactaccctcgaggaggaggcagcggcagccgccctcgcgtcgccgcccccggttcggtgcccgcggtcccggagaggaggtgccgccgccaccgccgctccccccctcccgctgccctcgggccgggctgggtcgagctgcgatgccctcggacttcatctcattgctcagcgcggacctagacctggaatcgcccaagtccctctactcgcgagattctctgaagttacacccatcacagaattttcatagagctggactattggaagaatctgtctatgatcttctcccaaaggagttacagttacctccatctagagaaacatctgtagcatcaatgagtcagacaagcggtggtgaggcaggctcgcctcctccagctgttgttgctgctggatttgcctctgaagcagggagtgtctgcattaaaaatgacctgtagttctctgcttcatagatgcttcttcagctccctcctcttcctccatgggcggtgcttgcagctcctttaccacctcttccagccctaccatttattctacctcagtcaccgacagcaaggctatgcaagtggagagctgctcctcagccgtgggggtaagtaacagaggggtaagtgaaaagcagttaaccagtaacacagttcagcagcatccatcaacaccgaagaggcacacagtcttgtacatctcaccaccacctgaggacttgctggataacagtcggatgtcctgccaggatgaggggtgtggattggaatctgagcagagctgcagtatgtggatggaggattccccctccaacttcagtaacatgagcaccagttcctacaatgataacactgaggtacctcgtaaatcacgaaaacgaaatccaaagcagaggccgggggtcaaacgacgagattgtgaagaatctaatatggatatatttgatgccgacagtgccaaagcacctcactatgtgctttctcagcttaccacggacaacaaaggcaactcaaaagcgggaaatggaacattggaaaaccaaaaaggaactggagtaaagaagagccctatgttgtgtggacaatatcctgttaaaagtgagggaaaggagctgaagatagttgtacaacctgagacacagcaccgagctcggtacctgactgagggcagccgtggctcagtgaaagatagaacacagcaaggctttcctacagtaaagctggaaggccataatgaacctgtagtgttgcaagtgtttgtgggcaacgactctggacgagtgaaaccacatggattttatcaggcctgcagagtaactggacgaaatacaactccttgcaaagaagtggacattgaaggcactactgttatagaagtcggccttgatcctagcaacaacatgacactggcggtggactgcgtagggatattgaaattgaggaatgctgatgtcgaagccagaataggaattgctggttccaagaagaaaagcactcgtgccagattggtttttcgagttaatatcatgaggaaagatggctccactttgacactgcaaacaccctcttctccaattttgtgtactcagccagcaggagtgccagaaatcttaaagaaaagcttgcatagctgttcagtgaaaggagaagaagaagtgtttttaatcggcaagaactttctgaaaggaactaaagttattttccaagaaaatgtttctgatgaaaactcttggaagtcagaagctgaaattgatatggaactatttcatcagaatcatcttattgtgaaggttcctccctatcatgaccaacatataactttgcctgtgtcagtgggaatatatgtagtgacaaatgctggaagatctcatgatgttcaaccattcacttacactccagacccagcagcagctggtgctttgaatgtaaatgtgaagaaggaaatatctagtccagcaagaccttgctcttttgaagaggccatgaaagcaatgaaaactactggatgtaatttagataaggtaaatattatccctaatgccctgatgactccactcataccaagcagtatgattaagagtgaagatgttactccaatggaagtaacagcagaaaaaagatcttccactatttttaagactacaaagtctgttggatcaactcagcaaacattagaaaacatctcaaacatagcaggaaatggctctttttcatcaccatcatcttcccacctaccttctgaaaatgaaaaacagcagcagattcagcccaaggcatacaacccagagaccctgacaactattcaaacccaggacatctcacagcctggtacttttccagcagtttctgcttctagtcagctgcccaacagcgatgcactattgcagcaggctacacagtttcagacaagagaaactcagtctagagagatattacagtcagatggtacagtggttaatttgtcacaactgactgaggcatcacaacaacagcagcagtcaccactacaagaacaagcacagactttacagcagcagatttcatcaaatatttttccatcaccaaatagtgtgagtcagcttcagaatactattcagcagctgcaagcagggagtttcacaggcagtactgctagtggcagcagtggaagtgttgacttggtccaacaagttttagaggcacagcagcagttatcttcagttttattttctgctccagatggtaatgagaatgttcaagagcagcttagtgcagatatttttcaacaagtcagtcaaattcagagtggtgtaagccctggaatgttttcctcaacagagccaacagtccataccagaccagataatttattacctggaagagctgaaagtgttcatccacagtctgaaaacacgttatctaatcaacagcagcagcagcagcagcaacagcaagtgatggaatcttcagccgcaatggtgatggagatgcaacagagtatctgccaggcagctgcccagattcagtcagagttattcccttcaactgcttcagcaaatggaaaccttcagcaatcgccagtttaccagcagacttctcacatgatgagtgcattgtctaccaatgaggatatgcaaatgcagtgtgaattgttttcttctcctcctgcagtttctggaaatgaaacttctacaactaccacacagcaggttgcaacccctggcactaccatgtttcagacatcaagttcaggagatggagaagaaactggaacacaagcaaaacagattcagaacagtgtctttcagaccatggtccaaatgcaacatagtggggacaatcaacctcaagttaaccttttttcatccacaaaaagtatgatgagtgttcagaatagtggtacccaacaacaaggtaatggtttattccagcaagggaatgagatgatgtcacttcaatctggaaattttttgcagcagtcttctcattcacaggcccaactttttcatcctcaaaatcctattgccgatgctcagaacctttcccaggaaactcaaggttctctctttcatagtccaaatcctattgtccacagtcagacttctacaacctcctctgaacaaatgcagcctccaatgtttcactctcaaagtaccattgctgtgttacagggctcttcagttcctcaagaccagcagtcaaccaacatatttctttcccagagtcccatgaataatcttcagactaacacagtagcccaagaagcattttttgcagcaccgaactcaatttctccacttcagtcaacatcaaacagtgaacaacaagctgctttccaacagcaagctccaatatcacacatccagactcctatgctttcccaagaacaggcacaacccccgcagcagggtttatttcagcctcaggtggccctgggctcccttccacctaatccaatgcctcaaagccaacaaggaaccatgttccagtcacagcactcaatagttgccatgcagagtaactctccatcccaggaacagcagcagcagcagcaacagcagcagcaacagcagcagcaacaacaacagagcattttattcagtaatcagaataccatggctacaatggcgtctccaaagcaaccaccaccaaacatgatattcaacccaaatcaaaatccaatggctaatcaggagcaacagaaccagtcaatttttcaccaacaaagtaacatggccccaatgaatcaagagcaacagcccatgcaatttcagagtcagtccacagtttcctcacttcagaacccaggtcctacccagtcggaatcatcacagacccccttgttccatagctctcctcagattcagttggtacaagggtcacctagttctcaagagcagcaagtaactctcttcttatctccagcatccatgtctgccttgcagaccagtataaatcaacaagatatgcaacagtctcctctttattcccctcagaacaacatgcctggaattcaaggagccacatcttcgcctcaaccacaggctactttatttcacaacacagcaggaggcacaatgaaccaactgcagaattctcctggctcatctcagcagacatcaggaatgttcttatttggcattcaaaataactgtagtcagcttttaacctctggaccagctacattgcctgatcagttgatggccataagtcagccaggccaaccacaaaacgagggccagccacctgtgacaacacttctttctcagcaaatgccagagaattctccactggcatcctctataaacaccaaccagaacatcgaaaagattgatttgcttgtttcattgcaaaaccaagggaacaacttgactggctccttttaactggatataaattccacgaagaaaatcctgattccaagatgtcctgagatcttgtggttccatgagaattattactttaaaaacaaaacaaaatataaaaaactgtgtttgagtaaactgatagattttactctgactgcaaaagagcacacctatgctgcttgttgcagtaactaaccaccaatgttaacatcttcatattttatattcctaataacagtgatgactgagaatctatttgagtttccagctggcagaattaattgttattattttcctaggcgcaatttccttaaacgtacagtttaaattcaaggctggaccactcagttattattgctattagaaaataatatatcatgtttacttttgttcttcattattttctttcctgcattgttttagtcaagtaatggcttttgaaaaagtaaagttcaataataactaaggctgtgatttttttcaatataaaaggcacagctgttggccaaagtgaaggaatcttttttcagttttattggagaaactgaagggtaacattctaacaagtaaactgtatgtgcagataaaagtactcttgatttaacacaaaggcagatgatacacttataaaactgggaacagctggaatgcttcttgattttattttttcagagagttgttagttctctgggtttctactaaggggtttagccataactgtgcatagaaaaataattatctgtaaaaaatgaaggggataatatatgataaattatgttctgatatcctcctacagtagtttaaattgacagaaaaatttgaatgttttcttcttaacccagtcttaggctggtattccctttttatatatatctatattacttttcacctctttttcactttactttagagaactattaatatactactggcttcatgaccctgtagcatctttggccactttaatctagggtgacctagcaatcctgcagcacagggcagagagtactgtcttaggaattattaggagttgattcctgagaaacaacacatttttccccatgaacggtgctgttctgaagtcttcaaatttttccctctaataggaaacagtataaattttaattaaaaaaaaaaggcaaactaaaatttcttgaaatatcacttctccctgatctgcagtgagtataaattcacttgtcacctcagtgctttacagtttgaagtggtcacttacctgatggttcccacaagccttaggctttacagggttgtatcattgacttaaaatgaagaattaacttgtgttacatctataaagagcaaaataacacactccagaacttggcagttgtagcattagttatacagttttgggtgttcttgccacccgtgggatgcctgcttctcactaccacctgtgtctggacacatgcttatgtctcattttccttttggcatgtggaaagctgtcaatgcagtgtaaggccaacgtgtgtgtggcttctatgtgttgagataatgttttggtatccttgtccgtttcatttattttttaagtgtacaaaaaataacctgttaattgttgaaggctacttttctgttctttttttttttttttttctatcctgtacatttagttgaactgtgcggaattgtggtgttggttttgtttacacagccagatttttccttctttttgttttgtgatgatcttcctttgttctttgaatgtgctcttttgtctttttctcttttttctcatgttttcttccctccacctccacccctttctttctttctctctctgattgagaggcattgaattacgttttcagtagtacaggcttcttgccgatatgaagggaacttttcagaaagagacctactctgggtcatttaattttgaatacagttttcaatcgttcaagttttggatggtttatatctaatgtgtgtttcatttttttggaaagctatattttgtatttaggaaatggtatactattttgctatttgtactgagtgagtacattggcataaatatagaaatttatatatatacatatatataaactattcttttttgccacacatttttgtggtaaatttgtgagtttgtctgatgttctaccacaacgtggcgtctgataacagtgagggggggtggggtttgttatgtctttattgagtatttaagtatcttttgaaacaaatgacctgttcatctgtggccattccatcaggcagttagttccttgatgtcagtagtgggctaaaggcagcttactgtgtgtttgctggagctttcactcagccaagtgttagagtcaggaaacccattgaggcaatggcgtcaaatggtgtttcacaagaatgagccattcagtctttgctcactatatatttaatattttattattgttgttattgttattattaattggctttctgtattctatgccttttatttataaagacactaagaaaacccatgtttgtaattttaataacatttttcccatcttgtaatatccagagctactttataaattctctgaaccaaaagtattttcctcagtgtatctcttctcccccagcccctattgggaaaaattacccagtatagttcaggttatgaggaggatcagccacacaatccagtgcttcagtttgaaaatgtaaaattctaaccctaaagtagggttggttgaaatttcagacaaagcaaacccagcaggtataaaaagtagtataaatacaaatctgtaagttatttttgaattttctgaacttttttctaagagattacataggagactaaagaaatctatctgttcaagttctaattaggatgattgttaatactgcactgtggatgaagtggcgactggcttgtgtgctgacttctgtggtttagcaagaggtttattgttatcaaatgctaattggcaatgccaagtcactgggaccaattttctgttttataatatctaagtttagaacagaatatatacctgaactgtagtggtttgatcggatggagacagaaaacccgatttttattctcataaattttgtggttatttatacaagggctgtgctatgctaccatattcttgttcaataataataggtttgttgttttttttacattgttaaatgttccttacccctaaaggtcaatgttaagtacaacattctgaaaatacaatttggctacgaagagtattcatcttctttgaagctcagtggttgatatttgtgctaataatgcaatttcctgattactgttacaagttatagctacatatgggagagactcagtgagccagcaaaggccatagaaacaacaatttattaaatgtatttatggcagaaggacctaaataaactgtgagccaccttttcttctttatattgttacatttaagtgttcttgctttcagcaactcacattaatgcttggagcttatctctttctctctctctctctctctctctgtgtgtgtgtgtgtatgtgtgtgtgtgtgtgtgtgtgtttccttattgtcattccattatatatccacaccaacatgggtgacgataattcaaagtcatattttgcctctaagcttgatcatgttacctttatgattaaagtatcatgttatttagccaatgcaaatctgttttaaaacaaatagtttaaaaaaagaacaagtttttaagggctttattatagaagaagtattaatgaaggactttccttcctccctccctttcctcccctccctgcctcccttcttcccttccatctccccctcctccctgccttctttgtttctccttcccttattcctccctccctcctttctcccttccttcctttcttccattcatccttccttgccttttatttttattttttgtaatatcacatgtgctgtagtttggaattttattctagtgcatttcttgctcatcagaacctcagctaatctacctaggaaaaatagtatcaaaggaaatgagaaagttgtatctgagtccctccagaactaagataattctttttgaccatttaagcctttataaatgcgttttgaccatttaagcctttataaatgcttgttttaggaaagtgaatctgttagatgcatcaacaaataatgaccaggacaaaacgatttaataattaaagtctcaaatcaccatggttatacattttcaccagaaatagtaatcttacaatttttcatttttctgatgaagatttctgttccaatatctgtttcctaatagattttttaaattaattagctttcctctgctttatgaccacaggttttatccctaaccgagacagctgtcttatatctgcatgccttagactgtgtggagggactccatgaagaaagaccataggttagaaaaataactcatagtatataccctagtaagtgggttagtagaatctcataacatgtattaaaaagaggttttcttctctgcttgtttgtgtcactagagcaaaattgtagagataatgctcataatgcagtaaatatcagaataatatctacaatatcatttgtggatggtcccaggtcccagtgctctagttactttacttctttttttttttttgagatggagtcttgctctgtctctcaggctagagcagtgtgcgatctcagctcactgcagcctccacctcccaggttcaagcgattctcctgcctcagcctcccaagtagccaggattacaggcaccctccactaggcccggctaattttttttgtatttttttagtagagatggggttttgccatgttggccaggctggtttcgaactcctaacctccagtgatccacctgcctcggcgtcccaaagtgctaggattacaggcatgagccaccacatccggcctaattacttctttaatccccatttatttttatgccattctagcctcatttattaataaaattatgtttttactttctctttcaggaaattttttaaattaatattttatatctagatctaatgctatggaaaagtgcctttttatcatttataatttcatttttcactatttccaaaaacacataaacaaatagtttcagtaggtcccagcttttactttttccatttaaaccttcttttctccatttcttccctttggcttaagaataaaagaaaaggtacattgctagaattgtttctttgggagagggtaaaagattacagaattagactgttcagcctttatataaactaaatttgtcttcatctcaaccagctaatggtaggtcttatctgaatactcatgagaattttagcatctgtgaaactccatgcaccagatgtgtgtaaatttcaggaagaaagtgttgaaagcattttctctgatgttaattagatggaaataaatcactaaaacatagtttaggtaaagcctgattatgccacttttttttaactagacagggcaaagttgtttatgttagtgtacttcttgtctatcctcagttaatttacctagacaaaaagtgtcaaaggaaatgagaaaaaggttatatctgactccctccagacctaagataattccttttgatcagatacagtcagatggagtgccttggtttttgttaattttgcctctattccagctccttaccacagcggtggtgcttaaagaaaggatcatcagcaacaggtcaggatagttctacctttgggatagggctgctttccccgtgctagtatttctgtgactgttagtggcactgaggactgcaaacttttatgcaatattcttaataccctattgatattatgcactttaatcattccaaagaagccaagaatgctgtatagtgatgattccttcctaatgaattcatcttaactatttagaatgttatgtcccttttcttttggatagccaacttggtataaatgttatatggatttttctaaaatgactatataggacttaagactttgaaatgtaatttacttataaggggaaataattatgctttagcacatcattttagaaacgtcacattttagaaacattcagcttgctaacctacatgtttgggaattcattaaaaccagttgtctatatattttgtgccatgtatataagaacattacaatatatctttttctacatatgtagtatgtgcaaccagtggttctcagagtatggttctcagcccaccagctagtatcagtatcacctgggaactagttagaaatgtaaattctttggccccatcccagacatactgagtcagaaactctggaatagggcccccgcaatctgttttcacaagccctccaggtgattctgatgcacactttaaagtttaggaaccactgggctaagactctgttgagatatagagtttttcttccactcagactgatatagttatacattgttcttcatgtaaattcagcttaacctggttatctataatcttttattggcaaaagttaattctcagtactgcctatagagatacagtgtattttatgtacatacacaattagtctaattcttgataattcagttaatttagtttggcattttcctaccacttactaaaaggtttacattaaatgactgatttaaatatataggtgcaatgttctatgtttattttaattgttatgacatttaagtagctaatataattgaccggtgctaaagtctcctgtttatccataaaatgggtacattatgggcagtgtaatacaagctttcttttcattgcctagtactttaccagcagaccacagttttgccctggctagaccaaccctcagaacaaaatcatcattccttgtatttatatttgtatctgagatagtaaacaagatggctggccaggtcaacatggcaccttaacttatttttttaataggtaaaacttcttcaaaagtagcttgctttgtataagaactaagctatcagtatagatatagctatccttggagcttatgtttcagacaagaattatttactaaaataaataataaacaagataatgcattatacaatttgggcatttctcgtttctcaagtgtatgcatcatggtaaatataaactaaccacaagataggtagattgattcatttcattttaatctccttgtgtaattcagtacctccataattgttctaatcttcttcccactgtttacaaattaccagttaattaactcgtgaaagaaaaattcacatatcagaataaaaataaatgtatactcactttataaaaatcaccactgctgtctttccttaatactagcagtggaaatgtaagtggcttactctacaaattttggtgctggcaaatacataggcaaactgttgggagctgctctagttacattcctcccttcttattccctttttctcttcctcactttattgcataacatattcctgtacccaaagcattctaccacagttctatttgactcccacttgtaataactcctttaaaaaattccatgtttaaccatatgaccctgcttgcttactcatattctccctccctctccccttcctttctctctcttccagaagtcatttgcctggtttgaaatattttgtagggattgcttattatattattttagctgatgaacctcaggacaacgtctacacacacacacatacatacacgcacacaaaatctcagctgttgaagagtgggcttggaatcagacttctgtgtccagtaaaaaactcctgcactgaagtcattgtgacttgagtagttacagactgattccagtgaacttgatctaatttcttttgatctaatgaatgtgtctgcttaccttgtttccttttaattgataagctccaagtagttgctaattttttgacaactttaaatgagtttcattcacttcttttacttaatgttttaagtatagtaccaataatttcattaacctgttctcaagtggtttagctaccattctgccatttttaatttttatttaattttatttgcttgagcacactgatcaaccactgaactgccttcttccattgtcctgcaatgatataagggttacatttttgtgtatatggctttcatagttgggatttcagagcactgataccagatattttcagtttgttctctgggggaatttcatttgcatctatgtttttagctatctgtgataacttgttaaatattaaaaagatattttgcttctattggaacatttgtatactcgcaactatatttctgtaaacagctgcagtcaaaaataaaacactgaaagttaaaaaaaaaaaaaaaaaaaa

SEQ ID NO: 17 - protein, 471 aa, Homo sapiens
MGGACSSFTTSSSPTIYSTSVTDSKAMQVESCSSAVGVSNRGVSEKQLTSNTVQQHPSTPKRHTVLYISPPPEDLLDNSRMSCQDEGCGLESEQSCSMWMEDSPSNFSNMSTSSYNDNTEVPRKSRKRNPKQRPGVKRRDCEESNMDIFDADSAKAPHYVLSQLTTDNKGNSKAGNGTLENQKGTGVKKSPMLCGQYPVKSEGKELKIVVQPETQHRARYLTEGSRGSVKDRTQQGFPTVKLEGHNEPVVLQVFVGNDSGRVKPHGFYQACRVTGRNTTPCKEVDIEGTTVIEVGLDPSNNMTLAVDCVGILKLRNADVEARIGIAGSKKKSTRARLVFRVNIMRKDGSTLTLQTPSSPILCTQPAGVPEILKKSLHSCSVKGEEEVFLIGKNFLKGTKVIFQENVSDENSWKSEAEIDMELFHQNHLIVKVPPYHDQHITLPVSVGIYVVTNAGRSHDVQPFTYTPDPAA

SEQ ID NO: 18 - protein, 16 aa, Homo sapiens
MAGPHPVIVITGPHEE

SEQ ID NO: 19 - DNA, 5994 bp, Homo sapiens
gcgctgcccgcctcgtccccaccccccaaccccccgcgcccgccctcggacagtccctgctcgcccgcgcgctgcagccccatctcctagcggcagcccaggcgcggagggagcgagtccgccccgaggtaggtccaggacgggcgcacagcagcagccgaggctggccgggagagggaggaagaggatggcagggccacgccccagcccatgggccaggctgctcctggcagccttgatcagcgtcagcctctctgggaccttggcaaaccgctgcaagaaggccccagtgaagagctgcacggagtgtgtccgtgtggataaggactgcgcctactgcacagacgagatgttcagggaccggcgctgcaacacccaggcggagctgctggccgcgggctgccagcgggagagcatcgtggtcatggagagcagcttccaaatcacagaggagacccagattgacaccaccctgcggcgcagccagatgtccccccaaggcctgcgggtccgtctgcggcccggtgaggagcggcattttgagctggaggtgtttgagccactggagagccccgtggacctgtacatcctcatggacttctccaactccatgtccgatgatctggacaacctcaagaagatggggcagaacctggctcgggtcctgagccagctcaccagcgactacactattggatttggcaagtttgtggacaaagtcagcgtcccgcagacggacatgaggcctgagaagctgaaggagccctggcccaacagtgacccccccttctccttcaagaacgtcatcagcctgacagaagatgtggatgagttccggaataaactgcagggagagcggatctcaggcaacctggatgctcctgagggcggcttcgatgccatcctgcagacagctgtgtgcacgagggacattggctggcgcccggacagcacccacctgctggtcttctccaccgagtcagccttccactatgaggctgatggcgccaacgtgctggctggcatcatgagccgcaacgatgaacggtgccacctggacaccacgggcacctacacccagtacaggacacaggactacccgtcggtgcccaccctggtgcgcctgctcgccaagcacaacatcatccccatctttgctgtcaccaactactcctatagctactacgagaagcttcacacctatttccctgtctcctcactgggggtgctgcaggaggactcgtccaacatcgtggagctgctggaggaggccttcaatcggatccgctccaacctggacatccgggccctagacagcccccgaggccttcggacagaggtcacctccaagatgttccagaagacgaggactgggtcctttcacatccggcggggggaagtgggtatataccaggtgcagctgcgggcccttgagcacgtggatgggacgcacgtgtgccagctgccggaggaccagaagggcaacatccatctgaaaccttccttctccgacggcctcaagatggacgcgggcatcatctgtgatgtgtgcacctgcgagctgcaaaaagaggtgcggtcagctcgctgcagcttcaacggagacttcgtgtgcggacagtgtgtgtgcagcgagggctggagtggccagacctgcaactgctccaccggctctctgagtgacattcagccctgcctgcgggagggcgaggacaagccgtgctccggccgtggggagtgccagtgcgggcactgtgtgtgctacggcgaaggccgctacgagggtcagttctgcgagtatgacaacttccagtgtccccgcacttccgggttcctgtgcaatgaccgaggacgctgctccatgggccagtgtgtgtgtgagcctggttggacaggcccaagctgtgactgtcccctcagcaatgccacctgcatcgacagcaatgggggcatctgtaatggacgtggccactgtgagtgtggccgctgccactgccaccagcagtcgctctacacggacaccatctgcgagatcaactactcggcgatccacccgggcctctgcgaggacctacgctcctgcgtgcagtgccaggcgtggggcaccggcgagaagaaggggcgcacgtgtgaggaatgcaacttcaaggtcaagatggtggacgagcttaagagagccgaggaggtggtggtgcgctgctccttccgggacgaggatgacgactgcacctacagctacaccatggaaggtgacggcgcccctgggcccaacagcactgtcctggtgcacaagaagaaggactgccctccgggctccttctggtggctcatccccctgctcctcctcctcctgccgctcctggccctgctactgctgctatgctggaagtactgtgcctgctgcaaggcctgcctggcacttctcccgtgctgcaaccgaggtcacatggtgggctttaaggaagaccactacatgctgcgggagaacctgatggcctctgaccacttggacacgcccatgctgcgcagcgggaacctcaagggccgtgacgtggtccgctggaaggtcaccaacaacatgcagcggcctggctttgccactcatgccgccagcatcaaccccacagagctggtgccctacgggctgtccttgcgcctggcccgcctttgcaccgagaacctgctgaagcctgacactcgggagtgcgcccagctgcgccaggaggtggaggagaacctgaacgaggtctacaggcagatctccggtgtacacaagctccagcagaccaagttccggcagcagcccaatgccgggaaaaagcaagaccacaccattgtggacacagtgctgatggcgccccgctcggccaagccggccctgctgaagcttacagagaagcaggtggaacagagggccttccacgacctcaaggtggcccccggctactacaccctcactgcagaccaggacgcccggggcatggtggagttccaggagggcgtggagctggtggacgtacgggtgcccctctttatccggcctgaggatgacgacgagaagcagctgctggtggaggccatcgacgtgcccgcaggcactgccaccctcggccgccgcctggtaaacatcaccatcatcaaggagcaagccagagacgtggtgtcctttgagcagcctgagttctcggtcagccgcggggaccaggtggcccgcatccctgtcatccggcgtgtcctggacggcgggaagtcccaggtctcctaccgcacacaggatggcaccgcgcagggcaaccgggactacatccccgtggagggtgagctgctgttccagcctggggaggcctggaaagagctgcaggtgaagctcctggagctgcaagaagttgactccctcctgcggggccgccaggtccgccgtttccacgtccagctcagcaaccctaagtttggggcccacctgggccagccccactccaccaccatcatcatcagggacccagatgaactggaccggagcttcacgagtcagatgttgtcatcacagccaccccctcacggcgacctgggcgccccgcagaaccccaatgctaaggccgctgggtccaggaagatccatttcaactggctgcccccttctggcaagccaatggggtacagggtaaagtactggattcagggcgactccgaatccgaagcccacctgctcgacagcaaggtgccctcagtggagctcaccaacctgtacccgtattgcgactatgagatgaaggtgtgcgcctacggggctcagggcgagggaccctacagctccctggtgtcctgccgcacccaccaggaagtgcccagcgagccagggcgtctggccttcaatgtcgtctcctccacggtgacccagctgagctgggctgagccggctgagaccaacggtgagatcacagcctacgaggtctgctatggcctggtcaacgatgacaaccgacctattgggcccatgaagaaagtgctggttgacaaccctaagaaccggatgctgcttattgagaaccttcgggagtcccagccctaccgctacacggtgaaggcgcgcaacggggccggctgggggcctgagcgggaggccatcatcaacctggccacccagcccaagaggcccatgtccatccccatcatccctgacatccctatcgtggacgcccagagcggggaggactacgacagcttccttatgtacagcgatgacgttctacgctctccatcgggcagccagaggcccagcgtctccgatgacactggctgcggctggaagttcgagcccctgctgggggaggagctggacctgcggcgcgtcacgtggcggctgcccccggagctcatcccgcgcctgtcggccagcagcgggcgctcctccgacgccgaggcccccacggccccccggacgacggcggcgcgggcgggaagggcggcagccgtgccccgcagtgcgacacccgggccccccggagagcacctggtgaatggccggatggactttgccttcccgggcagcaccaactccctgcacaggatgaccacgaccagtgctgctgcctatggcacccacctgagcccacacgtgccccaccgcgtgctaagcacatcctccaccctcacacgggactacaactcactgacccgctcagaacactcacactcgaccacactgcccagggactactccaccctcacctccgtctcctcccacgactctcgcctgactgctggtgtgcccgacacgcccacccgcctggtgttctctgccctggggcccacatctctcagagtgagctggcaggagccgcggtgcgagcggccgctgcagggctacagtgtggagtaccagctgctgaacggcggtgagctgcatcggctcaacatccccaaccctgcccagacctcggtggtggtggaagacctcctgcccaaccactcctacgtgttccgcgtgcgggcccagagccaggaaggctggggccgagagcgtgagggtgtcatcaccattgaatcccaggtgcacccgcagagcccactgtgtcccctgccaggctccgccttcactttgagcactcccagtgccccaggcccgctggtgttcactgccctgagcccagactcgctgcagctgagctgggagcggccacggaggcccaatggggatatcgtcggctacctggtgacctgtgagatggcccaaggaggagggccagccaccgcattccgggtggatggagacagccccgagagccggctgaccgtgccgggcctcagcgagaacgtgccctacaagttcaaggtgcaggccaggaccactgagggcttcgggccagagcgcgagggcatcatcaccatagagtcccaggatggaggacccttcccgcagctgggcagccgtgccgggctcttccagcacccgctgcaaagcgagtacagcagcatcaccaccacccacaccagcgccaccgagcccttcctagtggatgggctgaccctgggggcccagcacctggaggcaggcggctccctcacccggcatgtgacccaggagtttgtgagccggacactgaccaccagcggaacccttagcacccacatggaccaacagttcttccaaacttgaccgcaccctgccccacccccgccatgtcccactaggcgtcctcccgactcctctcccggagcctcctcagctactccatccttgcacccctgggggcccagcccacccgcatgcacagagcaggggctaggtgtctcctgggaggcatgaagggggcaaggtccgtcctctgtgggcccaaacctatttgtaaccaaagagctgggagcagcacaaggacccagcctttgttctgcacttaataaatggttttgctactgctaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2005/0100897 A1
(43) Pub. Date: May 12, 2005

(54) NFAT transcription factors in tumor progression

(76) Inventors: Alex Toker, Brookline, MA (US); Sebastien Jauliac, Paris (FR)

Correspondence Address: CLARK & ELBING LLP, 101 FEDERAL STREET, BOSTON, MA, 02110, US

(21) Appl. No.: 10/403,165

(22) Filed: Mar. 31, 2003

Related U.S. Application Data
(60) Provisional application No. 60/368,838, filed on Mar. 29, 2002.

(51) Int. Cl. C12Q 1/68

(52) U.S. Cl. 435/6

(57) ABSTRACT

The present invention provides therapeutic and diagnositic methods for neoplasia treatment. The invention also relates to determining the prognosis of a neoplasia or determining a treatment protocal. Further features of the invention are methods for identifying NFAT target genes that promote neoplasia progression.

### STATEMENT AS TO FEDERALLY SPONSORED RESEARCH

The present research was supported by a grant from the National Institutes of Health/National Cancer Institute (Number CA82695). The U.S. government has certain rights to this invention.

### CROSS-REFERENCE TO RELATED APPLICATIONS

This application claims benefit of U.S. provisional application No. 60/368,838, Mar. 29, 2002, which is hereby incorporated by reference.

### BACKGROUND OF THE INVENTION

The present invention features improved diagnostic and therapeutic methods for neoplasia treatment relating to NFAT (nuclear factor of activated T cells) nucleic acid molecules and polypeptides.

Approximately 1 million Americans will be diagnosed with neoplasia in 2003, and about half a million people in the United States will die of the disease. Worldwide, nearly one in five people will contract neoplasia during their lifetime. While improvements in neoplasia detection, diagnosis, and treatment have increased the survival rate for many types of neoplasia, only about 60 percent of people diagnosed with neoplasia are alive five years after treatment, making neoplasia the second leading cause of death in the United States.

Neoplasia includes a variety of related diseases, all of which are characterized by unrestrained and abnormal cell growth. Neoplasia cells that invade surrounding tissue or enter the bloodstream or lymphatic vessels form secondary tumors, or metastases, at a distance from the original tumor. Neoplasia that has metastasized is more difficult to treat and often has a poorer prognosis. Depending on the severity of the neoplasia (i.e., tumor size and invasiveness), a stage number is assigned, I, II, III, or IV. Stage I neoplasias are the least advanced and have the best prognosis. Stage II neoplasias typically include larger tumors and are associated with a somewhat poorer prognosis. Stage III and IV neoplasias have spread beyond their sites of origin and have the poorest prognosis.

### SUMMARY OF THE INVENTION

The present invention features improved diagnostic and therapeutic methods relating to NFAT nucleic acid molecules or polypeptides.

In one aspect, the invention generally features a method of diagnosing a patient as having, or having a propensity to develop, a neoplasia, the method involves determining the level of expression of an NFAT nucleic acid molecule in a patient sample, where an increased level of expression relative to the level of expression in a control sample, indicates that the patient has or has a propensity to develop a neoplasia. In one embodiment, the method further involves the step of determining the level of expression of a β4 integrin subunit relative to the level in a normal patient sample.

In a related aspect, the invention features a method of diagnosing a patient as having, or having a propensity to develop, a neoplasia, the method involves determining the level of expression of an NFAT polypeptide in a patient sample, where an increased level of expression relative to the level of expression in a control sample, indicates that the patient has or has a propensity to develop a neoplasia. In some embodiments, the method further involves the step of determining the level of expression of a β4 integrin subunit relative to the level in a normal patient sample. In other embodiments, the level of expression is determined in an immunological assay.

In a related aspect, the invention features a method of diagnosing a patient as having a neoplasia with a propensity to metastasize, the method involves determining the level of expression of an NFAT nucleic acid molecule in a patient sample, where an increased level of expression relative to the level of expression in a control sample, indicates that the patient has a neoplasia with a propensity to metastasize. In some embodiments, the invention further involves the step of determining the level of expression of a β4 integrin subunit relative to the level in a normal patient sample.

In another related aspect, the invention features a method of diagnosing a patient as having a neoplasia with a propensity to metastasize, the method involves determining the level of expression of an NFAT polypeptide in a patient sample, where an increased level of expression relative to the level of expression in a control sample, indicates that the patient has a neoplasia with a propensity to metastasize. In preferred embodiments, the method further involves the step of determining the level of expression of a β4 integrin subunit relative to the level in a normal patient sample. In other embodiments, the level of expression is determined in an immunological assay.

In another aspect, the invention features a method of monitoring a patient as having a neoplasia, the method involves determining the level of expression of an NFAT nucleic acid or polypeptide in a patient sample, where an alteration in the level of expression relative to the level of expression in a control sample indicates the severity of neoplasia in the patient. In preferred embodiments, the method monitors a patient being treated for a neoplasia. In other preferred embodiments, the method further involves the step of determining the level of expression of a β4 integrin subunit nucleic acid or polypeptide in a patient sample. In yet other preferred embodiments, the alteration is an increase, and the increase indicates an increased severity of neoplasia.

In another aspect, the invention features a method of determining the prognosis of a patient having, or having a propensity to develop, a neoplasia, the method involves determining the level of expression of an NFAT nucleic acid molecule in a patient sample, where an increased level of expression relative to the level of expression in a control sample, indicates that the patient has a poor prognosis. In some embodiments, the method further involves the step of determining the level of expression of a β4 integrin subunit relative to the level in a normal patient sample.

In another aspect, the invention features a method of determining the prognosis of a patient having, or having a propensity to develop, a neoplasia, the method involves determining the level of expression of an NFATpolypeptide in a patient sample, where an increased level of expression relative to the level of expression in a control sample, indicates that the patient has a poor prognosis. In one preferred embodiment, the method further involves the step of determining the level of expression of a β4 integrin subunit relative to the level in a normal patient sample. In another embodiment, the level of expression is determined in an immunological assay.

In yet another aspect, the invention features a method of diagnosing a patient as having a neoplasia with a propensity to metastasize, the method involves determining the level of expression of an NFAT nucleic acid molecule in a patient sample, where an increased level of expression relative to the level of expression in a control sample, indicates that the patient has a neoplasia with a propensity to metastasize. In some embodiments, the method further involves the step of determining the level of expression of a β4 integrin subunit relative to the level in a normal patient sample.

In preferred embodiments of the above aspects, the control sample is a normal patient sample, a reference sample taken from the patient, or a sample taken from a patient having a non-metastatic neoplasia. In other preferred embodiments, the patient sample is a blood or tissue sample.

In another aspect, the invention features a diagnostic kit for the diagnosis of a neoplasia in a patient containing a nucleic acid or amino acid sequence, or fragment thereof, selected from the group consisting of an NFAT-1, NFAT-4, or NFAT-5 nucleic acid, or fragment thereof, and any combination thereof. In preferred embodiments, the diagnostic kit further contains a β4 integrin subunit.

In yet another aspect, the invention features a method of treating or preventing a neoplasia, the method involves administering to a patient in need of such treatment an effective amount of a pharmaceutical composition containing an NFAT polypeptide having a dominant negative mutation selected from the group consisting of NFAT-1, NFAT-4, or NFAT-5.

In a related aspect, the invention features a method of treating or preventing a neoplasia, the method involves administering to a patient in need of such treatment an effective amount of a pharmaceutical composition containing a nucleic acid molecule selected from the group consisting of an NFAT-1, NFAT-4, and NFAT-5, complementary nucleic acids, or fragments thereof. In preferred embodiments, the nucleic acid molecule encodes an NFAT polypeptide having a dominant negative mutation. In other preferred embodiments, the nucleic acid molecule is a double stranded RNA that interferes with the expression of an NFAT nucleic acid molecule.

In another related aspect, the invention features a method of treating or preventing a neoplasia, the method involves administering to a patient in need of such treatment an effective amount of a pharmaceutical composition that decreases expression of an NFAT polypeptide.

In another aspect, the invention features a method of identifying a candidate compound that inhibits a neoplasia, the method involves contacting a cell that expresses an NFAT nucleic acid molecule with a candidate compound, and comparing the level of expression of the nucleic acid molecule in the cell contacted by the candidate compound with the level of expression in a control cell not contacted by the candidate compound, where a decrease in expression of the NFAT nucleic acid molecule identifies the candidate compound as a candidate compound that inhibits a neoplasia. In one embodiment, the decrease in expression is a decrease in transcription. In another embodiment, the decrease in expression is a decrease in translation.

In another aspect, the invention features a pharmaceutical composition including an NFAT polypeptide or portion thereof, having a dominant negative mutation, where the NFAT is selected from the group consisting of an NFAT-1, NFAT-4, and NFAT-5, formulated in a pharmaceutically acceptable carrier.

In a related aspect, the invention features a pharmaceutical composition containing an NFATnucleic acid molecule, or portion thereof, selected from the group consisting of an NFAT-1, NFAT-4, and NFAT-5, formulated in a pharmaceutically acceptable carrier. In preferred embodiments, the nucleic acid molecule encodes an NFAT polypeptide having a dominant negative mutation. In other preferred embodiments, the nucleic acid molecule encodes a double stranded RNA that interferes with the expression of an NFAT nucleic acid molecule or polypeptide.

In another aspect, the invention features a method of identifying a candidate compound that inhibits a neoplasia, the method involves contacting a cell that expresses an NFAT polypeptide with a candidate compound, and comparing the level of expression of the polypeptide in the cell contacted by the candidate compound with the level of polypeptide expression in a control cell not contacted by the candidate compound, where a decrease in the expression of the NFAT polypeptide identifies the candidate compound as a candidate compound that inhibits a neoplasia.

In a related aspect, the invention features a method of identifying a candidate compound that inhibits a neoplasia, the method involves contacting a cell that expresses an NFAT polypeptide with a candidate compound, and comparing the biological activity of the polypeptide in the cell contacted by the candidate compound with the level of biological activity in a control cell not contacted by the candidate compound, where a decrease in the biological activity of the NFAT polypeptide identifies the candidate compound as a candidate compound that inhibits a neoplasia.

In some embodiments of the preceding aspects, the decrease in expression is assayed using an immunological assay, an enzymatic assay, or a radioimmunoassay.

In another aspect, the invention features a microarray containing at least two NFAT nucleic acid molecules, or fragments thereof, bound to a solid support, where at least 90% of the nucleic acid molecules on the support are mitochondrial energy metabolism nucleic acid molecules. In some embodiments, the array contains NFAT nucleic acid molecules, or fragments thereof, selected from the group consisting of NFAT-1, NFAT-4, or NFAT-5 nucleic acid molecules. In other preferred embodiments, the array further contains a β4 integrin subunit nucleic acid molecule.

In a related aspect, the invention features a microarray containing at least two NFAT polypeptides, or fragments thereof, bound to a solid support, where at least 90% of the polypeptides on the support are NFAT polypeptides. In preferred embodiments, the array further contains a β4 integrin subunit.

In another aspect, the invention features a collection of primer sets, each of the primer sets containing at least two primers that bind to an NFAT nucleic acid molecule that encodes a polypeptide selected from the group consisting of an NFAT-1, NFAT-4, and NFAT-5, under high stringency conditions, the collection containing at least two different primer sets.

In another aspect, the invention features a method of identifying a candidate compound that inhibits a neoplasia, the method involves a) contacting a cell containing an NFAT nucleic acid molecule present in an expression vector that includes a reporter constructed; b) measuring the expression of the reporter gene; c) comparing the level of reporter gene expression in the cell contacted with the candidate compound with a control cell not contacted with the candidate compound, where a decrease in the level of the reporter gene expression identifies the candidate compound as a candidate compound that inhibits a neoplasia.

In a related aspect, the invention features a method for identifying an NFAT target gene that promotes neoplasia progression, the method involves a) providing a first cell that fails to express an NFAT polypeptide; b) providing a second cell having an invasive phenotype that expresses an NFAT polypeptide; and c) identifying differentially expressed genes in the second cell relative to the first cell, where a differentially expressed gene is an NFAT target gene. In some embodiments, the level of expression of the target gene is increased in the second cell relative to the first cell, and this increase identifies the target gene as a target gene that is transcriptionally activated by NFAT biological activity. In other embodiments, the level of expression of the target gene is decreased in the second cell relative to the first cell, and this decrease identifies the target gene as a target gene that is transcriptionally inhibited by NFAT biological activity. In preferred embodiments, the differentially expressed genes are identified using a microarray.

In another aspect, the invention features a method for identifying an NFAT regulated protein that promotes neoplasia progression, the method involves a) providing a first cell that fails to express an NFAT polypeptide; b) providing a second cell having an invasive phenotype that expresses an NFAT polypeptide; c) identifying differentially expressed proteins in the second cell relative to the first cell, where a differentially expressed protein is an NFAT regulated protein. In some embodiments, the level of the protein is increased in the second cell relative to the first cell, and this increase identifies the protein as a protein that is upregulated in response to NFAT biological activity. In other embodiments, the level of the protein is decreased in the second cell relative to the first cell, and this decrease identifies the protein as a protein that is downregulated in response to NFAT biological activity. In preferred embodiments, the differentially expressed proteins are identified using a microarray.

In another aspect, the invention features a method for identifying an NFAT target gene that promotes neoplasia progression, the method involves a) hybridizing a candidate nucleic acid molecule with an NFAT polypeptide dimer; b) determining binding of the candidate nucleic acid molecules and the dimer; c) transforming a cell with the candidate nucleic acid molecule; and d) determining the phenotype of the transformed cell in a matrigel cell invasion assay; where an invasive phenotype identifies the candidate nucleic acid molecule as an NFAT target gene. In some embodiments, the cell expresses an endogenous NFAT nucleic acid molecule. In other embodiments, the NFAT dimer contains an NFAT binding partner selected from the group consisting of bZIP class proteins, Rel family members, and GATA factor family polypeptides. In related embodiments, the NFAT dimer contains an NFAT binding partner selected from the group consisting of AP-1, ATF, CREB, GATA, MAF, MEF, NF-kappaB and Rel. In other preferred embodiments, the determining is by microarray analysis or chromatin immunoprecipitation.

In another aspect, the invention features a method for treating the progression of neoplasia, the method involves introducing an NFAT consensus DNA-binding nucleic acid sequence, the nucleic acid sequence is double stranded, the sequence inhibits NFAT biological activity, regardless of length of the nucleic acid sequence. In preferred embodiments, a consensus NFAT DNA binding sequence is selected from a group consisting of SEQ ID NO:1, SEQ ID NO:2, and SEQ ID NO:3.

In preferred embodiments of any of the above aspects, an NFAT nucleic acid molecule or NFAT polypeptide is an NFAT-1, NFAT-4, or NFAT-5 nucleic acid molecule (e.g., DNA, cDNA, RNA, double stranded RNA, anti-sense RNA, or dominant negative) or polypeptide (e.g., a wild-type, mutant, or dominant negative).

In other preferred embodiments of the above aspects, a β4 integrin subunit is α6β4. In yet other preferred embodiments of the above aspects, a neoplasia is a sarcoma, carcinoma, or plasmacytoma.

By “antisense” is meant a nucleic acid sequence, regardless of length, that is complementary to the coding strand or mRNA of an NFAT gene. Preferably, the antisense nucleic acid is capable of decreasing the expression of NFAT in a cell by at least 10%, 20%, 30%, 40%, or more preferably by at least 50%, 60%, 70%, or 75%, or even by as much as 80%, 90%, or 95% relative to an untreated control cell. The antisense nucleic acid may contain a modified backbone, for example, phosphorothioate, phosphorodithioate, or other modified backbones known in the art, or may contain non-natural internucleoside linkages.

By “assaying” is meant analyzing the effect of a treatment, be it chemical or physical, administered to whole animals or cells derived there from. The material being analyzed may be an animal, a cell, a lysate or extract derived from a cell, or a molecule derived from a cell. The analysis may be, for example, for the purpose of detecting altered gene expression, altered RNA stability, altered protein stability, altered protein levels, or altered protein biological activity. The means for analyzing may include, for example, enzymatic assays, immunoprecipitation, phosphorylation assays, and methods known to those skilled in the art for detecting nucleic acids and polypeptides.

By “candidate compound” is meant any nucleic acid molecule, polypeptide, or other small molecule, that is assayed for its ability to alter gene or protein expression levels, or the biological activity of a gene or protein by employing one of the assay methods described herein. Candidate compounds include, for example, peptides, polypeptides, synthesized organic molecules, naturally occurring organic molecules, nucleic acid molecules, and components thereof.

By “consensus NFAT DNA binding sequence” is meant any sequence recognized and specifically bound by an NFAT polypeptide. In one embodiment, an NFAP polypeptide recognizes, binds, and transactivates NF-κB-like consensus sequences that are found in the promoters of TNF-α, IL-8, E-selectin, GM-CSF and IL-2. In another embodiment, a consensus NFAT DNA binding sequence is SEQ ID NO. 1, SEQ ID NO. 2, or SEQ ID NO. 3.

By “detectably-labeled” is meant any means for marking and identifying the presence of a molecule, e.g., a polypeptide or fragment thereof, or a nucleic acid molecule. Methods for detectably-labeling a molecule are well known in the art and include, without limitation, radioactive labeling (e.g., with an isotope such as ³²P or ³⁵S) and non-radioactive labeling (e.g., chemiluminescent labeling or fluorescein labeling).

By “differentially expressed” is meant a difference in the expression level of a nucleic acid molecule or polypeptide. This difference may be either an increase or a decrease of at least 10%, 20%, 30%, 40%, 50%, 60%, 70%, 80%, 90% or 95% in expression, relative to a reference or to control expression.

By “dimer” is meant a protein complex of two proteins. In one embodiment, a dimer is an NFAT monomer in complex with any transcriptional binding partner. Dimers include homodimers and heterodimers. In one embodiment, a homodimer is composed of an NFAT monomer, such as NFAT-5, bound to another NFAT-5. In another embodiment, a heterodimer is composed of an NFAT molecule, such as NFAT-1 or NFAT-4, bound with, for example, AP-1 (activator protein-1), NF-κB (nuclear factor B), GATA, or MAF (musculoaponeurotic fibrosarcoma). Guidance relating to assaying protein interaction or protein function may be found in, for example, Ausubel et al. (Current Protocols in Molecular Biology, John Wiley & Sons, New York, 2000).

By “effective amount” is meant an amount sufficient to arrest, ameliorate, or inhibit the continued proliferation, growth, or metastasis (e.g., invasion, or migration) of a neoplasia.

By “promoter” is meant a nucleic acid sequence sufficient to direct transcription. A promoter may be cell type-specific, tissue-specific or inducible by external signals or agents; such elements may be located in the 5′ or 3′ regions of the native gene. Preferred promoters direct transcription of an NFAT protein or nucleic acid molecule in an epithelial cell; such promoters include, without limitation, promoters from the following genes: keratin-14, keratin-19, Her2/Neu/erbB2, kallikrein-10, Gro-alpha, and MDM-2. Other preferred promoters direct the transcription of an NFAT protein or nucleic acid in an embryonal cell.

By “greater than naturally-occurring amount of NFAT nucleic acid molecule, NFAT protein, or NFAT biological activity” is meant an increased amount of NFAT nucleic acid molecule, NFAT protein, or NFAT biological activity, that is at least 10%, 20%, 30%, or 40% greater, preferably is 50%, 60%, 75%, or 80% greater, or is even 90%, 100%, 200%, 500%, or 1000% greater than the amount of NFAT protein or NFAT activity that is naturally present within a particular cell or sample. A greater than naturally-occurring amount of NFAT protein may be generated by expressing a heterologous NFAT protein in a cell or sample. Alternatively, a NFAT protein may be expressed in a cell or sample using a heterologous promoter, by using multiple copies of the endogenous gene, or by exploiting a mutation in the NFAT gene or elsewhere in a cell chromosome that results in increased NFAT expression or activity.

By a “heterologous nucleic acid molecule” is meant a nucleic acid molecule (e.g., a cDNA, DNA or RNA molecule) that is not normally present in a cell or sample obtained from a cell. This nucleic acid may be from another organism, or it may be, for example, an mRNA molecule that is not normally expressed in a cell or sample.

By a “heterologous promoter” is meant a promoter that regulates the expression of a nucleic acid with which it is not normally associated. A “heterologous promoter” may be, for example, a viral promoter (e.g., a cytomegalovirus (CMV) promoter, a herpes simplex virus thymidine kinase promoter, or an adenovirus E1B promoter), a bacterial promoter, or a mammalian promoter, such as the β-globin promoter.

By “metastatic potential” is meant the ability of a neoplasia cell to metastasize, from the site of neoplasia origin to at least one other site, tissue, or organ.

By “metastasis” is meant the spread of at least one neoplasia cell from a site of origin to at least one additional site (tissue, or organ). Often metastasis occurs by way of the lymph system or bloodstream. Markers that may be associated with metastatic tumor growth include, but are not limited to, carcinoembryonic antigen (CEA), CA 19-9, p53 mutations, CA 15-3 (a mucin-like membrane glycoprotein), estrogen, progesterone receptors, c-erbB-2, and cathepsin-D. Metastatic neoplasias include, but are not limited to, neoplasias that have metastasized to the brain, bones, kidney, liver, lungs, or lymph nodes.

By “microarray” is meant an organized collection of at least two nucleic acid molecules or polypeptides affixed to a solid support. In some embodiments, a nucleic acid microarray is composed of oligonucleotides having at least a portion (e.g., 10, 15, 18, 20, 21, 22, 23, 24, 25, 26, 27, 28, 29, 30, 40, 50, 60, 70, 80, 90, or 100 nucleotides) of two or more nucleic acid sequences. A polypeptide microarray contains at least a portion of a polypeptide (e.g., 10, 20, 30, 40, 50, 75, or 100 amino acids). A microarray contains at least 2, 5, 10, 25, 50, 75, 100, 150, 200, 250, or 300 polypeptide or nucleic acid molecule members. In one embodiment, the nucleic acid molecules are arranged in a grid where the location of each nucleic acid remains fixed to aid in identification of the individual nucleic acids. A microarray may include, for example, DNA representing all, or a subset, of the open reading frames of an organism. Preferably, the DNA is derived from a region of the genome that shares limited homology to other regions of organism's genome.

By “modulating” is meant increasing or decreasing. In one example, the expression of an NFAT protein, NFAT nucleic acid molecule, or the biological activity of such a molecule is increased or decreased by at least 5%, 10%, or 15%; more preferably, the increase or decrease is 20%, 30%, or 40%; and most preferably, the increase or decrease is of 50%, 60%, 70%, 80%, 90%, or even 95%, relative to the levels observed in a cell that naturally expresses NFAT. Preferably, the NFAT protein expression, NFAT nucleic acid molecule expression, or NFAT biological activity is decreased.

By “mutation” is meant any alteration in a naturally-occurring or reference nucleic acid or amino acid sequence. A mutation can be an insertion, deletion (e.g., of at least 1, 2, 3, 4, 5, 6, 7, 8, 9, 10, 20, 30, 40, 50, 60, 70, 89, 90, 100 or more nucleotides or amino acids), frameshift mutation, silent mutation, nonsense mutation, or missense mutation. Preferably, the amino acid sequence encoded by the nucleic acid sequence has at least one amino acid alteration relative to a naturally-occurring sequence. In one embodiment, an NFAT nucleic acid molecule having a mutation encodes a dominant negative NFAT polypeptide.

By “neoplasia” is meant a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as sarcomas, carcinomas, or plasmacytomas (malignant tumor of the plasma cells).

By “NFAT nucleic acid molecule” is meant a polynucleotide (e.g., DNA, cDNA, RNA, mRNA antisense RNA, or double-stranded RNA), or fragment thereof, that shares homology with at least a portion of a nucleic acid sequence that encodes an NFAT polypeptide (e.g., a wild-type or mutant NFAT polypeptide, such as NFAT-1, NFAT-4, NFAT-5 polypeptides). For example, an NFAT-1 nucleic acid molecule is substantially identical to at least a portion of GenBank Accession Number NM_{—}012340 (SEQ ID NO:4 nucleic acid and SEQ ID NO:5 amino acid), U43342, or U43341; an NFAT-4 nucleic acid molecule is substantially identical to at least a portion of GenBank Accession Number NM_{—}004555 (SEQ ID NO:6 nucleic acid and SEQ ID NO:7 amino acid); and an NFAT-5 nucleic acid molecule is substantially identical to at least a portion of GenBank Accession Number AF163836 (SEQ ID NO:8 nucleic acid and SEQ ID NO:9 amino acid), AJ243299, AJ243298, NM_{—}006599, Nm_{—}138714, AF134870, or NP_{—}619728.

By “NFAT polypeptide” is meant an NFAT protein, or fragment thereof, such as a wild-type NFAT-1, NFAT-4, or NFAT-5 polypeptide, or a mutant NFAT-1, NFAT-4, or NFAT-5 (e.g., a dominant negative NFAT-1, -4, or -5). A wild-type NFAT polypeptide has the biological activity of a naturally-occuring NFAT polypeptide. Previously known biological activities of NFAT polypeptides include, but are not limited to, transactivation of target genes; interaction with specific transcription factor binding partners (e.g., AP-1, NF-κB, GATA, MAF, bZIP (basic leucine zipper) class proteins, Rel family members, and GATA); DNA binding (e.g., binding to nucleic acid sequences containing TGGAAANNYNY, where N is any nucleotide and Y represents any pyrimidine). The bZIP class of transcription factors includes, but is not limited to Fos-Jun family members, Maf, Mef, CREB, and ATF. Rel family members include Rel and NF-κB. As shown herein, NFAT polypeptides can induce an increased-migration phenotype when expressed in a cell. This NFAT biological activity can be assayed in a cell migration or cell invasion assay as described herein.

By “integrin” is meant a receptor polypeptide for an extracellular matrix ligand, composed of two subunits, α and β. One preferred α subunit is the α₆ subunit. The α6 subunit nucleic acid sequence is substantially identical to GenBank Accession No. NM_{—}000210 (SEQ ID NO:10 nucleic acid and SEQ ID NO:11 amino acid). An α subunit can combine with several integrin β subunits. One preferred integrin β subunit is the β4 subunit encoded by a nucleic acid sequence substantially identical to GenBank Accession No. NM_{—}000213 (SEQ ID NO:19).

By “NFAT dominant negative” is meant an NFAT polypeptide, or the nucleic acid molecule encoding it, having a mutation that selectively blocks or inhibits the biological activity of a wild-type NFAT protein, (e.g., the ability of an NFAT polypeptide to promote transcription, neoplasia progression, cell migration or cell invasion). Various NFAT dominant negative polypeptides and the nucleic acid molecules encoding them are known to the skilled artisan (see, for example, Lopez-Rodriguez, C. et al. Immunity 15: 47-58, 2001; Aramburu, J. et al. Science 285: 2129-33, 1999, and Northrop, J. P. et al. Nature 369:497-502, 1994). For example, an NFAT dominant negative mutant may have a mutation or deletion in the carboxyl terminus required for DNA-binding; or have a mutation or deletion that blocks dimerization of endogenous NFAT-5. In one embodiment, an NFAT-5 dominant negative mutant is DBD5. DBD5 comprises amino acids 175-471 (SEQ ID NO:14) of NFAT5a (Genebank Accession No.: NP_{—}619728, SEQ ID NO:15 protein; NM_{—}138714 SEQ ID NO:16 nucleic acid). In another embodiment, a dominant negative mutant is ND5. ND5 comprises amino acids 1-471 (SEQ ID NO:17) of NFAT5a (Genebank Accession No.: NP_{—}619728). The dominant negative mutant inhibits NFAT biological activity by at least 10%, 20%, 30%, 40%, 50%, 60%, 70%, 80%, 90%, or 95%.

By “operably linked” is meant that a nucleic acid molecule and one or more regulatory sequences (e.g., a promoter) are connected in such a way as to permit expression and/or secretion of the product (i.e., a polypeptide) of the nucleic acid molecule when the appropriate molecules (e.g., transcriptional activator proteins) are bound to the regulatory sequences.

By “pharmaceutically acceptable carrier” is meant a carrier that is physiologically acceptable to the treated mammal while retaining the therapeutic properties of the compound with which it is administered. One exemplary pharmaceutically acceptable carrier is physiological saline. Other physiologically acceptable carriers and their formulations are known to one skilled in the art and described, for example, in Remington: The Science and Practice of Pharmacy (20th ed., ed. A. R. Gennaro AR., 2000, Lippincott Williams & Wilkins).

By “portion” is meant a part of a whole. A portion may comprise at least 10%, 20%, 30%, 40%, 50%, 60%, 70%, 80%, 90%, or 95% of the entire length of an amino acid or nucleic acid sequence. A portion may also comprise at least 10, 20, 30, 40, 50, 60, 70, 80, 90, 100, 200, 300, 400, 500, 600, 700, 800, 900, or 1000 nucleotides or amino acids.

By “positioned for expression” is meant that the DNA molecule is positioned adjacent to a DNA sequence, which directs transcription or translation of the sequence (i.e., facilitates the production of, e.g., CAR receptor polypeptide).

By “neoplasia progression” is meant the spreading (e.g., invasion, migration, metastasis) or growth of a neoplasia. Neoplasia progression can occur in the presence or the absence of treatment. During neoplasia progression, alterations in the molecular control of cell growth and survival occur. Such alterations typically include genetic alterations that result in the neoplasia cell assuming a more aggressive growth phenotype.

By “prognosis” is meant a forecase of the probable outcome of neoplasia.

When a patient is diagnosed with neoplasia, the patient's prognosis is determined in part by the stage of the neoplasia. While the determinants of neoplasia stage vary, depending on the type of neoplasia, a higher stage number generally at least a portion of indicates an increase in the severity of the neoplasia and a poorer patient prognosis. Stage I neoplasias are generally the least severe, and have the best prognosis. Stage I neoplasias are typically small and localized, generally they have not penetrated the mucosal layer. Stage II neoplasias are somewhat more severe and have a somewhat poorer prognosis. Stage II neoplasias are typically larger in size, and may have penetrated into neighboring tissues beyond the site of origin. Stage III neoplasias have typically spread from the site of origin to nearby lymph nodes. In stage IV, neoplasia has typically spread from the site of origin to other tissues on organs.

By “protein” or “polypeptide” is meant a chain of amino acids, regardless of length or post-translational modification (for example, glycosylation or phosphorylation).

By “reporter gene” is meant a gene whose expression may be assayed; such genes include, without limitation, those encoding glucuronidase (GUS), luciferase, chloramphenicol acetyl transferase (CAT), green fluorescent protein (GFP), alkaline phosphatase, and β-galactosidase.

By “RNA interference (RNAi)” is meant the administration of a ribonucleic acid sequence, regardless of length, that inhibits the expression of an NFAT gene. The RNA may be an antisense RNA or a double stranded RNA. Typically, the administered RNA contains one strand that is complementary to the coding strand of an mRNA of an NFAT gene. RNAi is a form of post-transcriptional gene silencing initiated by the introduction of double-stranded RNA (dsRNA) or antisense RNA. Preferably, RNAi is capable of decreasing the expression of NFAT in a cell by at least 10%, 20%, 30%, or 40%, more preferably by at least 50%, 60%, or 70%, and most preferably by at least 75%, 80%, 90%, 95% or more. The double stranded RNA or antisense RNA is at least 10, 20, or 30 nucleotides in length. Other preferred lengths include 40, 60, 85, 120, or more consecutive nucleotides that are complementary to a NFAT mRNA or DNA, and may be as long as a full-length NFAT gene or mRNA. The double stranded nucleic acid may contain a modified backbone, for example, phosphorothioate, phosphorodithioate, or other modified backbones known in the art, or may contain non-natural internucleoside linkages. In one preferred embodiment, short 21, 22, 23, 24, or 25 nucleotide double stranded RNAs are used to down regulate NFAT expression. Such RNAs are effective at down-regulating gene expression in mammalian tissue culture cell lines (Elbashir et al., Nature 411:494-498, 2001, hereby incorporated by reference). The further therapeutic effectiveness of this approach in mammals was demonstrated in vivo by McCaffrey et al. (Nature 418:38-39. 2002). The nucleic acid sequence of an NFAT gene can be used to design small interfering RNAs that will inactivate an NFAT gene and that may be used, for example, as therapeutics to treat a variety of neoplasias.

By “substantially identical” is meant a polypeptide or nucleic acid exhibiting at least 75%, but preferably 85%, more preferably 90%, most preferably 95%, or even 99% identity to a reference amino acid or nucleic acid sequence. For polypeptides, the length of comparison sequences will generally be at least 20 amino acids, preferably at least 30 amino acids, more preferably at least 40 amino acids, and most preferably 50 amino acids. For nucleic acids, the length of comparison sequences will generally be at least 60 nucleotides, preferably at least 90 nucleotides, and more preferably at least 120 nucleotides.

Sequence identity is typically measured using sequence analysis software with the default parameters specified therein (e.g., Sequence Analysis Software Package of the Genetics Computer Group, University of Wisconsin Biotechnology Center, 1710 University Avenue, Madison, Wis. 53705). This software program matches similar sequences by assigning degrees of homology to various substitutions, deletions, and other modifications. Conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine.

By “substantially pure polypeptide” is meant a polypeptide that has been separated from the components that naturally accompany it. Typically, the polypeptide is substantially pure when it is at least 60%, by weight, free from the proteins and naturally occurring organic molecules with which it is naturally associated. Preferably, the polypeptide is a polypeptide that is at least 75%, more preferably at least 90%, and most preferably at least 99%, by weight, pure. A substantially pure polypeptide may be obtained, for example, by extraction from a natural source (e.g., a fibroblast) by expression of a recombinant nucleic acid encoding the polypeptide, or by chemically synthesizing the protein. Purity can be measured by any appropriate method, e.g., by column chromatography, polyacrylamide gel electrophoresis, or HPLC analysis.

A protein is substantially free of naturally associated components when it is separated from those contaminants, which accompany it in its natural state. Thus, a protein, which is chemically synthesized or produced in a cellular system different from the cell from which it naturally originates, will be substantially free from its naturally associated components. Accordingly, substantially pure polypeptides not only include those derived from eukaryotic organisms but also those synthesized in E. ColE or other prokaryotes.

By a “therapeutic amount” is meant an amount of a compound, alone or in combination with known therapeutics, that is sufficient to inhibit neoplasia growth, progression, or metastasis in vivo. The effective amount of an active compound(s) used to practice the present invention for therapeutic treatment of neoplasms (i.e., neoplasia) varies depending upon the manner of administration, the age, body weight, and general health of the subject. Ultimately, the attending physician or veterinarian will decide the appropriate amount and dosage regimen. An effective amount of an NFAT therapeutic for the treatment of neoplasia is as little as 0.005, 0.01, 0.02, 0.025, 0.05, 0.075, 0.1, 0.133 mg per dose, or as much as 0.15, 0.399, 0.5, 0.57, 0.6, 0.7, 0.8, 1.0, 1.25, 1.5, 2.0 or 2.5 mg per dose. The dose may be administered once a day, once every two, three, four, seven, fourteen, or twenty-one days. The amount administered to treat neoplasia is based on the activity of the therapeutic compound. It is an amount that is sufficient to effectively reduce cell proliferation, tumor size, neoplasia progression, or metastasis. It will be appreciated that there will be many ways known in the art to determine the therapeutic amount for a given application. For example, the pharmacological methods for dosage determination may be used in the therapeutic context.

By “transcriptional activation” is meant the activation of a transcription factor, for example, an NFAT polypeptide. NFAT5 transcriptional activition is induced, for example, by α6β4 integrin signaling. Increased NFAT polypeptide transcriptional activity leads to increased NFAT cellular expression, increased nuclear localization, increased target gene expression and the promotion of carcinoma invasion.

By “transgene” is meant any piece of nucleic acid that is inserted by artifice into a cell, or an ancestor thereof, and becomes part of the genome of the animal, which develops from that cell. Such a transgene may include a gene, which is partly or entirely heterologous (i.e., foreign) to the transgenic animal, or may represent a gene homologous to an endogenous gene of the animal.

By “treatment regimen” is meant the method or combination of methods used to decrease or ameliorate the progression, proliferation, metastasis, or severity of a neoplasia. A neoplasia treatment regimen typically includes chemotherapy, hormone therapy, immunotherapy, or radiotherapy. Aggressive treatment regimens are employed in patients having a poor prognosis, such as patients having metastatic neoplasia, or having a neoplasia with a propensity to metastasize (e.g., having an increased level of NFAT polypeptide or nucleic acid molecule expression relative to the level of NFAT expression in a control sample from a normal patient, or a patient having a non-metastatic neoplasia). In one embodiment, an aggressive treatment regimen features the administration of an increased dosage of a chemotherapeutic, hormone therapeutic, immunotherapeutic, or radiotherapeutic relative to the dosage to a patient with a poor prognosis relative to the dosage typically administered to a patient with a good prognosis. An aggressive treatment regimen generally features the use of therapeutics or dosages typically associated with increased toxicity or an increased risk of adverse side-effects.

Other features and advantages of the invention will be apparent from the following description of the desirable embodiments thereof, and from the claims.

### BRIEF DESCRIPTION OF DRAWINGS

FIGS. 1A-1F show that NFATS are expressed and active in carcinoma cells. FIG. 1A shows an electrophoretic mobility shift assay. Nuclear extracts from MDA-MB-435 breast carcinoma cells stably transfected with either vector alone (6D2, 6D7), with a mutated integrin Δ₄ subunit (Δ₄.ΔCYT (3C12)), with wild-type β₄ (3A7, 5B3), or parental cells were incubated with a ³²P-labeled NFAT probe (top panel) or a ³²P-labeled AP-1 probe (bottom panel), in duplicate, and separated on 6% polyacrylamide gel. The positions of the specific NFAT- and AP-1 probe complexes are indicated. The position of a non-specific band (NS, right) is also shown. FIG. 1B shows the results of a supershift experiment. The specific anti-NFAT-1 antibody, (clone 67.1), or control non-immune serum (N.I. serum) were pre-incubated with nuclear extracts of the 5B3 clone prior to the EMSA with NFAT probe. The position of the supershifted NFAT/antibody/probe complex is shown. The first lane shows nuclear extract incubated with NFAT probe only. FIG. 1C is a graph. The transcriptional activity of NFAT was measured by transiently transfecting a luciferase-NFAT reporter construct (NFAT-luc) into the MDA-MB-435 clones and parental cells. Luciferase activity is shown as relative luciferase units. FIG. 1D shows an immunoblot of total cell lysates from MDA-MB-435 (parental, vector alone (6D2), β₄.ΔCYT and wild-type β₄ (3A7)). The cell lysates were immunoblotted with the NFAT-1-specific antibody (clone 67.1) (top left panel), or with an antibody against the p85 subunit of PI 3-K (α-p85, bottom left panel). Nuclear extracts from the same cells were also prepared and immunoblotted with the same NFAT-1-specific antibody (right panel). FIG. 1E shows an immunoblot of total cell lysates from 3A7 and 5B3 clones that were resolved by SDS PAGE and immunoblotted with an anti-NFAT-1 antibody (α-NFAT-1, clone 67.1). FIG. 1F shows an immunoblot of total cell lysates from MDA-MB-231 and clone A cells that were resolved by SDS PAGE and immunoblotted with anti-NFAT-1 (oc-NFAT-1, clone 67.1, left panel), anti-NFAT-4 (α-NFAT-4, right panel) or anti-p85 (α-p85, bottom panels) antibodies. The positions of multiple phosphorylated NFAT species (arrowheads, right) and molecular mass markers (kDa, left) are indicated. The data are representative of 3 independent experiments, and expressed as a mean±SEM, performed in triplicate (FIG. 1C).

FIGS. 2A-2B are graphs showing that NFAT activity is both necessary and sufficient to drive carcinoma invasion. FIG. 2A is a graph showing the results of a Matrigel chemoinvasion assay of MDA-MB-435 β4⁺-expressing cells (3A7). FIG. 2B is a graph showing the results of a Matrigel chemoinvasion assay of MDA-MB-231cells. Cells were transiently transfected with DN-NFAT, the VIVIT peptide (GFP-VIVIT), the constitutively active NFAT mutant (NFAT-mSPx3), or constitutively active calcineurin alleles ΔCnA and CnB, either alone or co-transfected with VIVIT. Following the transfection protocol, the ability of the transfected cells to invade Matrigel was assayed. The data are shown as the relative invasion of NFAT transfected cells compared to vector alone-transfected cells (100%). The data shown are mean values (±SEM) of 2 experiments performed in triplicate.

FIGS. 3A-3C show the role of NFAT-5 in mediating carcinoma invasion. FIG. 3A is a graph showing luciferase levels in MDA-MB-435 cells expressing vector alone, β4.ΔCYT (3C12), or wild-type β4⁺ (3A7 clone) that were transiently transfected with an NFAT-5-luciferase reporter construct (NFAT-5-luc). Luciferase activity is shown as relative luciferase units. FIG. 3B is an immunoblot that shows NFAT5 or p85 expression in cell lysates of Clone A, MDA-MB-435 cells transfected with 3A7, β4.ΔCYT, or 6D2, parental control cells, and MDA-MB-231 cells. The lysates were resolved by SDS PAGE and immunoblotted with anti-NFAT-5 (α-NFAT-5, top panel) or anti-p85 (α-p85, bottom panel) The position of the 175 kDa and 90 kDa markers are indicated (left). FIG. 3C shows three graphs of cell invasion assays. Dominant negative NFAT-5 constructs (ND5, DBD5), or control inactive DBD5 (DIM1) were transiently transfected into MDA-MB-435 β4⁺-expressing cells (3A7 clone), MDA-MB-231 cells and clone A cells, as indicated. The ability of the transfected cells to invade Matrigel was then assayed and the data are shown as the relative invasion of NFAT-5-transfected cells compared to vector alone-transfected cells (100%).

FIGS. 4A-4D are graphs showing that NFAT-5 transcriptional activity is induced in an α6β4-dependent manner. FIG. 4A is a graph showing NFAT-5 transcriptional activity in control parental cells, β4 (3A7), and β4.ΔCYT (3C12) MDA-MB-435 transfectants that were transiently transfected with NFAT-5-luc. The cells were grown in complete media for twenty-four hours, trypsinized, incubated with anti-α6 (clone 135-13C) for 30 minutes, then plated onto secondary antibody-coated plates, either in the presence (+) or absence (−) of NIH 3T3 conditioned medium (CM), for twenty-four hours. As a positive control, cells were also stimulated with PMA (phorbol 12-myristate 13-acetate) and ionomycin (100 nM each) for twenty-four hours. Following stimulation, cells were lysed and NFAT-5 transcriptional activity was assayed. The data are presented as fold induction in NFAT-5 transcriptional activity compared to control, unstimulated cells (−). FIG. 4B is a graph showing transcriptional activity in Clone A cells that were transiently transfected with the NFAT-5-luc reporter and incubated (as described in FIG. 4A) in the presence (+) or absence (−) of CM, or with control PMA/ionomycin alone. NFAT-5 transcriptional activity was measured as described above. FIG. 4C is a graph showing NFAT-5 transcriptional activity in MDA-MB-435 cells expressing β4⁺ (3A7 clone) that were transiently transfected with GFP-ND5 and NFAT-5-luc. The cells were treated with CM, anti-α6, and PMA/ionomycin as described above. FIG. 4D is a graph showing NFAT-5 transcriptional activity in MDA-MB-435 β4⁺ transfectants (3A7) that were transiently transfected with NFAT-5-luc, grown in complete media for twenty-four hours, trypsinized, and plated onto laminin-1-coated plates (lanes 3 and 4) or uncoated plates (lanes 1, 2 and 5) in the presence (+) or absence (−) of CM for twenty-four hours. NFAT-5 transcriptional activity was measured as described above. In all cases, the data are representative of 2 independent experiments performed.

FIGS. 5A-5B are graphs and immunoblots showing the effects of NFAT1 and NFAT5 over expression on transfected cells. Parental MDA-MB-435 β4-negative cells were transiently transfected with wild-type NFAT-1 (FIG. 5A) tagged with the HA epitope (NFAT-1) or with wild-type NFAT-5 (FIG. 5B) tagged with the myc epitope (NFAT-5) or control vector alone (vector). Following the transfection protocol, cells were assayed for their ability to migrate or to invade Matrigel. Relative migration/invasion were determined by comparing the amount of migration/invasion obtained from NFAT transfectants to that obtained from cells transfected with vector alone, which was assigned a value of 100%. An aliquot of transfected cells was lysed and nuclear extracts were resolved by SDS PAGE and immunoblotted with anti-HA or anti-myc. The data shown are mean values (±SEM) of 3 experiments performed in triplicate.

FIGS. 6A-6R are photomicrographs showing NFAT-1, NFAT-5, and β4 integrin expression detected by immunofluorescence in human breast carcinoma tissue sections. All sections were obtained from four different individuals (patients 1-4) with grade III invasive ductal breast carcinoma. Sections from two different individuals (patient 1, FIGS. 6A-6C, patient 2, FIGS. 6D-6F) were stained with anti-cytokeratin (FIGS. 6A, 6D) and anti-NFAT-1 (FIG. 6B, 6E). The merged images are shown in FIGS. 6C and 6F. Arrows indicate cells that express NFAT-1, but not cytokeratin (FIGS. 6E, 6F). A separate carcinoma section and surrounding cells from patient 2 was stained with anti-NFAT-1 (FIG. 6G) and anti-CD3 (FIG. 6H). The overlay (FIG. 61) reveals infiltrating lymphocytes that express both NFAT-1 and CD3 (arrows). A section from patient 3 was stained with anti-cytokeratin (FIG. 6J) and NFAT-5 antibodies (FIG. 6K). The overlay is shown in FIG. 6L. β4 integrin expression in invasive ductal carcinoma is shown in FIGS. 6M-R. A section from patient 2 was stained with anti-β4 (FIG. 6m) and anti-NFAT-1 (FIG. 6N). A section from patient 4 was stained with anti-β4 (FIG. 6P) and anti-NFAT-5 (FIG. 6Q). The merged images are shown in panels 6O and 6R respectively. Magnification is 20× for FIG. 6A-6L and 60x for panels 6M-6R.

FIGS. 7A-7H are graphs showing the results of FACScan flow cytometry. Twenty-four hours after transfection of the indicated plasmids into Clone A, MDA-MB-231, and MDA-MB-435 cells, as indicated, cells were trypsinized, washed in PBS and labeled for 20 minutes with anti-α6, anti-β1, and anti-β4 antibodies directly coupled to phycoerythrin. After labeling, the cells were washed twice in PBS and analyzed for α6, β1, and β4 expression on a FACScan flow cytometer (Becton Dickinson). The data are plotted as relative cell number against log fluorescence intensity for each of the indicated antibodies. Thus, expression of the various indicated NFAT mutants does not alter the expression of the surface α6β4 and α6β1 integrins, such that the alteration in carcinoma cell migration is specifically linked to NFAT action, and not to alteration in integrin expression or function.

FIG. 8 is a table showing the percentage of annexin-V positive cells in a transfected cell population. Twenty-four hours following transfection of the indicated plasmids into MDA-MB-435 β4⁺ (3A7) cells, the cells were trypsinized, washed in PBS, and labeled for thirty minutes with an anti-annexin-V antibody coupled to phycoerythrin, according to the manufacturer's instructions. After labeling, cells were washed twice in PBS and annexin-V positive cells analyzed on a FACScan flow cytometer (Becton Dickinson) gated for transfected cells (GFP-positive). The data are presented as the percentage of annexin-V positive cells in the transfected population. The data demonstrate that expression of the indicated NFAT mutant proteins does not lead to increased cell death, and therefore increased apoptosis cannot account for the altered carcinoma migration and invasion observed when NFAT function is increased or decreased.

FIG. 9 is a graph showing luciferase expression in MDA-MB-435 β4 transfectants (3A7) that were transiently transfected with the NFAT-luc reporter. Twenty-four hours later the cells were trypsinized, incubated with anti-α6 antibody for 20 minutes, and plated onto secondary antibody-coated plates for twenty-four hours, either in the presence (+) or absence (−) of conditioned media. Control cells were also stimulated with PMA/ionomycin (100 nM each). The cells were then lysed and assayed for NFAT-luciferase activity, and luciferase activity was plotted as relative luciferase units. As shown, activation or engagement of the α6β4 integrin does not specifically lead to an increase in the intrinsic transcriptional activity of NFAT-1 in carcinoma cells, rather it is the increase in NFAT-1 expression (FIG. 1) that mediates the increased invasive phenotype in α6β4-expressing cells. In the same cells, the control stimuli PMA+ionomycin does increase NFAT-1 transcriptional activity, indicating that a distinct signaling pathway is responsible for α6β4-dependent NFAT-1 activation.

FIG. 10 is a graph showing luciferase expression in MDA-MB-435 β4 transfectants that were transiently transfected with the NFAT-luc reporter construct, and treated with either FK506 (10 nM) or vehicle alone (methanol) for 1 hour, prior to stimulation with PMA+ionomycin (100 nM each) for twenty-four hours. Cells were then lysed and NFAT-luciferase activity was determined, and plotted as relative luciferase units. Therefore, in carcinoma cells, the Ca ²⁺/calcineurin pathway leading to NFAT activation does not appear to be necessary for induction of NFAT-1 activation, although this pathway is present because the control stimuli PMA/ionomycin do stimulate NFAT-1 activity and this can be blocked with FK506. This is reminiscent of previous studies, which have also indicated that two distinct mechanisms are required for NFAT-1 activation in T-cells, one mechanism that is FK506-sensitive (and thus Ca²⁺/calcineurin-dependent), and one that is not.

### DETAILED DESCRIPTION

The present invention features improved diagnostic and therapeutic methods for neoplasia treatment.

As reported in more detail below, we have discovered that NFAT is involved in promoting carcinoma invasion. We provide evidence that both NFAT1 and the recently identified NFAT5 isoform are expressed in invasive human ductal breast carcinomas. Similarly, both NFAT isoforms are expressed at high levels and are constitutively active in cell lines derived from human breast and colon carcinomas. This activity correlates with the expression of the α6β4 integrin and importantly, is sufficient to promote carcinoma invasion. In addition, the transcriptional activity of NFAT5 is induced by α6β₄ clustering in the presence of chemoattractants, leading to enhanced migration of cells. These observations show for the first time that NFATs are novel targets of α6β₄ integrin signaling and demonstrate that they are both necessary and sufficient to promote carcinoma invasion, highlighting a novel function for this family of transcription factors in human neoplasia.

Overview

The transition of a benign, non invasive tumor to an invasive and metastatic one involves alterations in gene expression and intracellular signaling pathways. Integrins, receptors for extracellular matrix ligands, are critical regulators of the invasive phenotype. Specifically, the α6β₄ integrin has been linked with epithelial cell motility, cellular survival, and carcinoma invasion, which are hallmarks of metastatic tumors. The α6β₄ integrin in cells derived from various human carcinomas mediates carcinoma invasion by amplifying several intracellular signaling pathways, such as the phosphoinositide 3-OH kinase (PI 3-K) and the Met receptor tyrosine kinase pathways. Here, we investigate the involvement of NFAT in promoting carcinoma invasion.

NFAT Polypeptides

An NFAT polypeptide typically comprises (from the amino-terminus): an amino terminal acidic/hydrophobic transactivation domain; a calcineurin binding domain, also referred to as the NFAT homology region; a nuclear localization sequence; a DNA binding domain; and a carboxy-terminal transactivation domain. Between the calcineurin-binding domain and the DNA binding domain are a number of serine-rich and serine/proline-rich regions that are able to be phosphorylated. NFAT-1 and NFAT-4 mRNA and polypeptides are expressed in hematopoietic tissues and cells, as well as in brain (Plyte et al., J. Biol. Chem. 276:14350-8, 2001), muscle (Boss et al., J. Biol. Chem. 273:9664-71, 1998), kidney, placenta, and endothelium. NFAT-5 is ubiquitously distributed.

Some NFAT polypeptides (e.g., NFAT-5) form homodimers, while others (e.g., NFAT-1, 2, 3, or 4) form heterodimers with various transcriptional binding partners (e.g., bZIP class proteins, Rel family members, or GATA). Dimerization of NFAT polypeptides and their subsequent binding to target DNA typically results in an increase in the transcription of a target gene. NFAT target genes include cytokines (e.g., GM-CSF, IFN-γ, interleukins -2, -4, -5, and -13) and lymphocyte markers (e.g., CD40L and CTLA-4). NFAT polypeptides also are able to recognize and transactivate NF-κB-like consensus sequences that are found in the promoters of TNF-α, IL-8, E-selectin, GM-CSF and IL-2. The expression of an NFAT target gene is increased when consensus NFAT DNA binding sequences are adjacent to DNA binding sequences of their transcriptional binding partners.

NFATs are Expressed and Active in Carcinoma Cells.

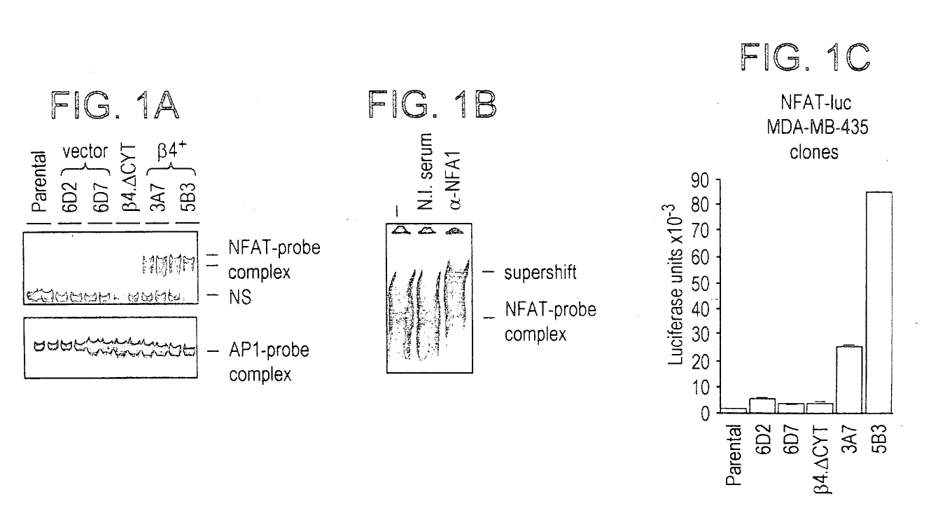

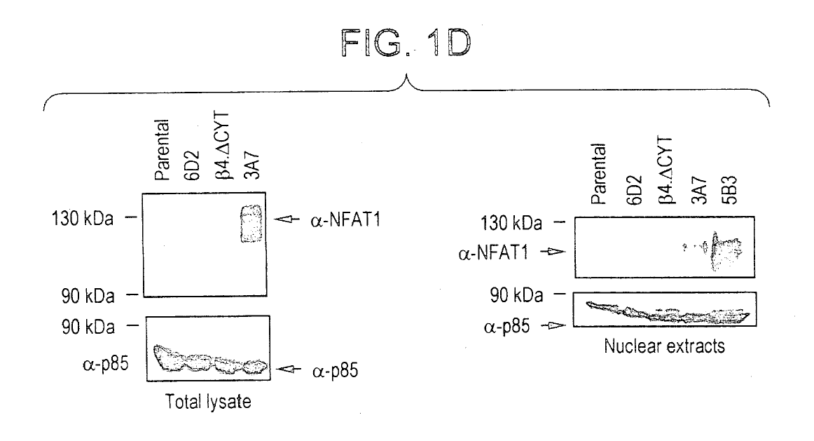

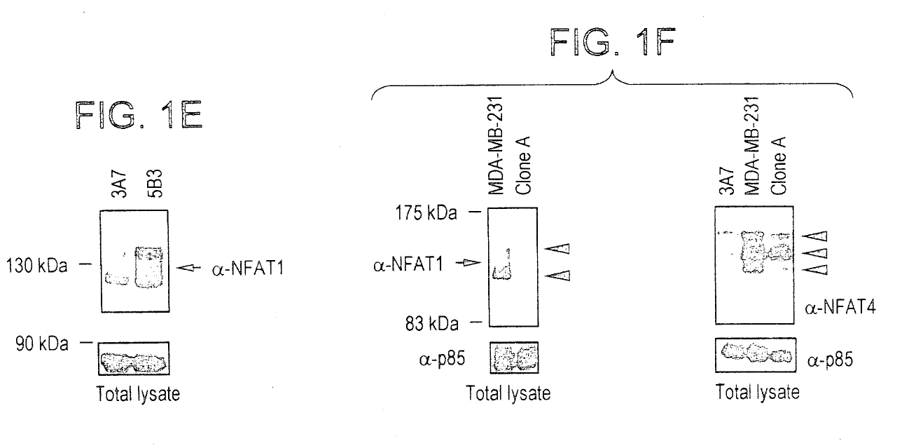

Signaling through β4 integrin has been linked with epithelial cell motility (Rabinovitz & Mercurio, J. Cell Biol. 139:1873-1884, 1997). We sought to determine the relevance of NFAT expression in carcinoma cells which express α6β4. For this purpose, we used MDA-MB-435 breast carcinoma cells which endogenously express the α6β1 integrin, and that have been stably transfected with the β4 subunit leading to expression of functional α6β4 (β4+), or a mutated β4 subunit lacking the β4 cytoplasmic domain (β4.ΔCYT) that is unable to activate downstream signaling pathways (Shaw et al., Cell 91:949-960, 1997). MDA-MB-231 breast carcinoma and Clone A colon carcinoma cells that endogenously express α6β4 were also used. MDA-MB-435 cells were found to constitutively express nuclear NFAT. This was determined in an electrophoretic mobility shift assay (EMSA) using a specific ³²P-labeled nucleotide probe for the NFAT DNA recognition sequence (FIG. 1A). Our results indicated that the NFAT complex was predominantly found in two separate clones of cells expressing α6β4, whereas in parental, β4.ΔCYT or vector alone-transfected cells, this complex was present at lower levels. In many instances, NFATs co-operate with other transcription factors, most notably AP-1 (Fos/Jun) to mediate gene expression, although there are examples of AP-1-independent NFAT function (Macian et al., Embo. J. 19:4783-95, 2000). AP-1 was detected in all MDA-MB-435 clones. Unlike NFAT, its DNA-binding activity was not increased in cells expressing α6β4 (FIG. 1A). We therefore focused on determining the relevance of increased nuclear NFAT in these cells.

A supershift experiment was performed by pre-incubating nuclear extracts with an anti-NFAT-1 antibody, or non-immune serum (FIG. 1B). This experiment showed that the NFAT-1 isoform was present in nuclear extracts of β4⁺-expressing cells. The NFAT-1 detected in MDA-MB-435 β4⁺-expressing cells was transcriptionally active with a high basal activity, measured using an NFAT luciferase reporter construct (FIG. 1C). Expression of NFAT-1 in MDA-MB-435 cells was confirmed by immunoblotting total cell lysates with an NFAT-1 specific antibody (Feske et al., J. Immunol. 165:297-305, 2000). NFAT-1 protein resolved as multiple bands on SDS-PAGE (FIG. 1D, left panel), indicating the presence of differentially phosphorylated species (Okamura et al., Mol. Cell 6:539-50, 2000), and correlating with an observed increase in nuclear NFAT-1 in MDA-MB-435 β4⁺-expressing cells (FIG. 1D, right panel). Two separate clones that expressed the α6β4 integrin showed distinct NFAT transcriptional activities and interestingly, the clone with the highest NFAT transcriptional activity (5B3) also showed the highest expression of total cellular NFAT-1 (FIG. 1E) and nuclear NFAT (FIG. 1D, right panel).

To determine that the presence of NFAT-1 in β4⁺-expressing-cells was not restricted to clonally derived cell lines, expression of NFAT-1 was also evaluated in MDA-MB-231 breast cancer cells and Clone A colon cancer cells that endogenously expressed α6β4. Immunoblotting experiments revealed that NFAT-1 was also expressed at high levels in MDA-MB-231 cells, whereas clone A cells predominantly expressed NFAT-4 (FIG. 1F). As a control for equivalent protein loading, lysates were also probed with an antibody that recognizes the p85 subunit of PI3-K. Equal amounts of p85 were detected in all cell lines tested (FIGS. 1D, 1E, and 1F). We demonstrated that three distinct epithelial carcinoma cell lines expressed NFATs, and that this correlated with expression of the β4 subunit.

NFAT Activity Drives Carcinoma Invasion

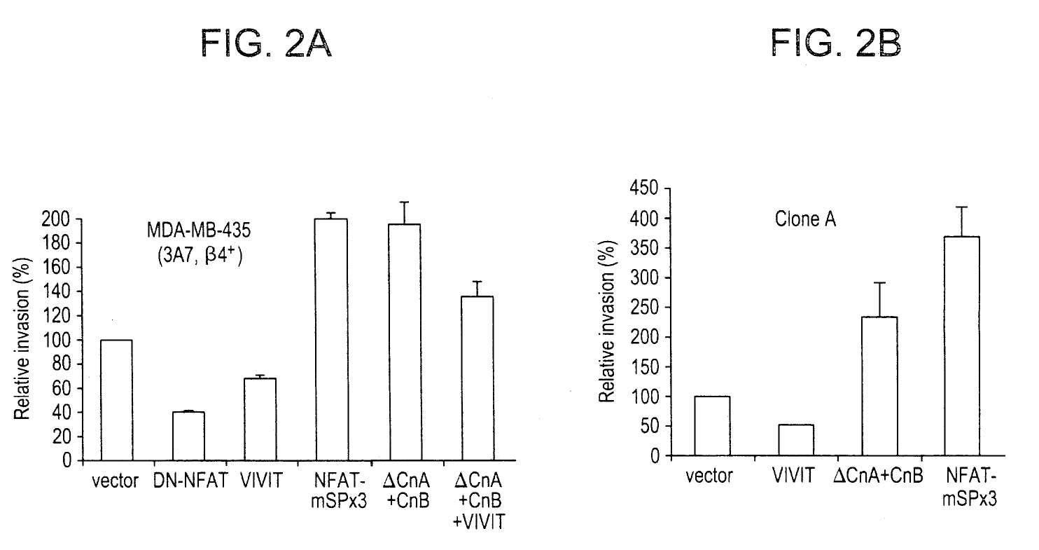

We used a molecular genetic approach to investigate the role of NFAT in carcinoma invasion. This approach involved expressing dominant negative alleles of NFAT as well as constitutively active mutants in transfected carcinoma cells. Two distinct NFAT dominant negative mutants were used, one deleted in the carboxyl terminus in the sequence necessary for DNA-binding (Northrop et al., Nature 369:497-502, 1994) and the VIVIT peptide (SEQ ID NO:18) (Aramburu et al., Science 285:2129-33, 1999), which blocks the ability of calcineurin to dephosphorylate and activate NFAT. Both mutants reproducibly inhibited carcinoma invasion when transfected into MDA-MB-435 β4⁺-transfectants, as measured in a standard Matrigel chemoinvasion assay (FIG. 2A). Conversely, transfection of an activated NFAT allele, mutated in three serines required for NFAT nuclear export, caused an increase in the invasive phenotype (FIG. 2A). Similarly, a constitutively active calcineurin allele, containing a mutant catalytic A subunit and a wild type regulatory B subunit (Milan et al., Cell 79:437-47, 1994; O'Keefe et al., Nature 357:692-4, 1992), was also able to significantly increase carcinoma invasion. This invasion was also inhibited by the VIVIT peptide.

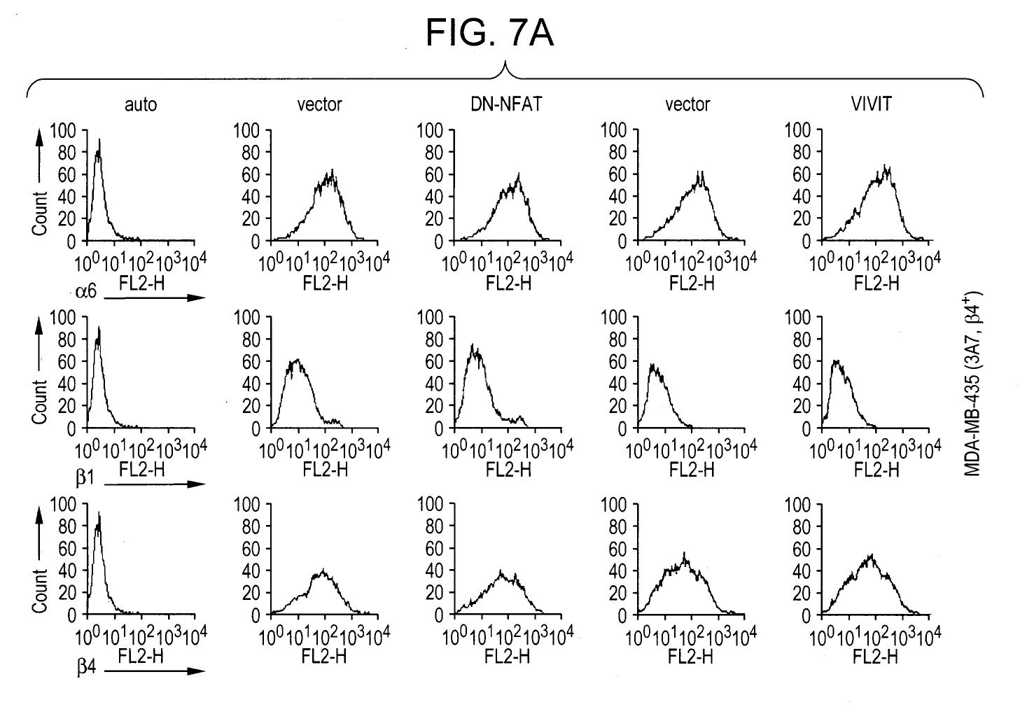

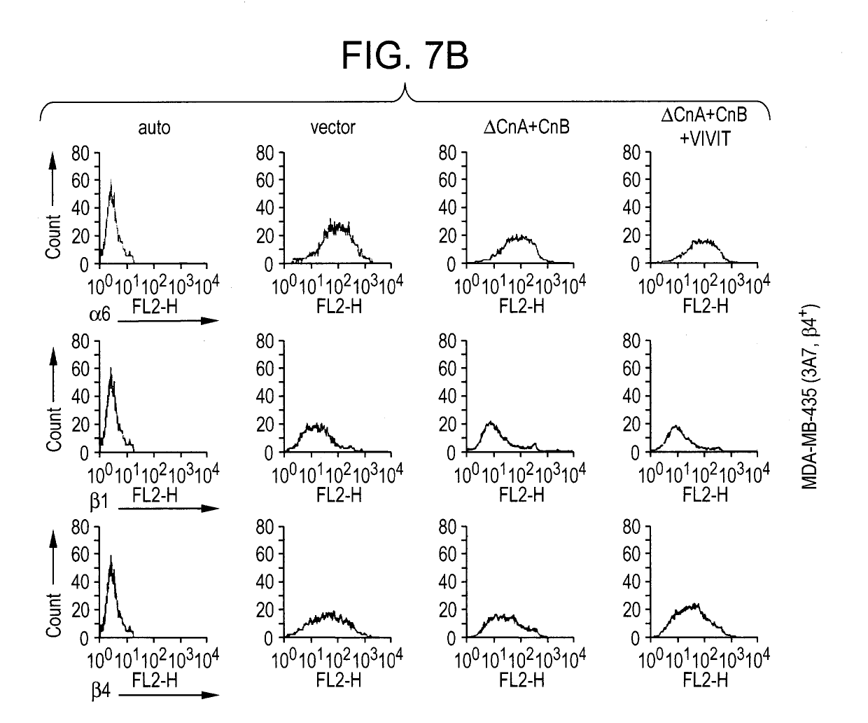

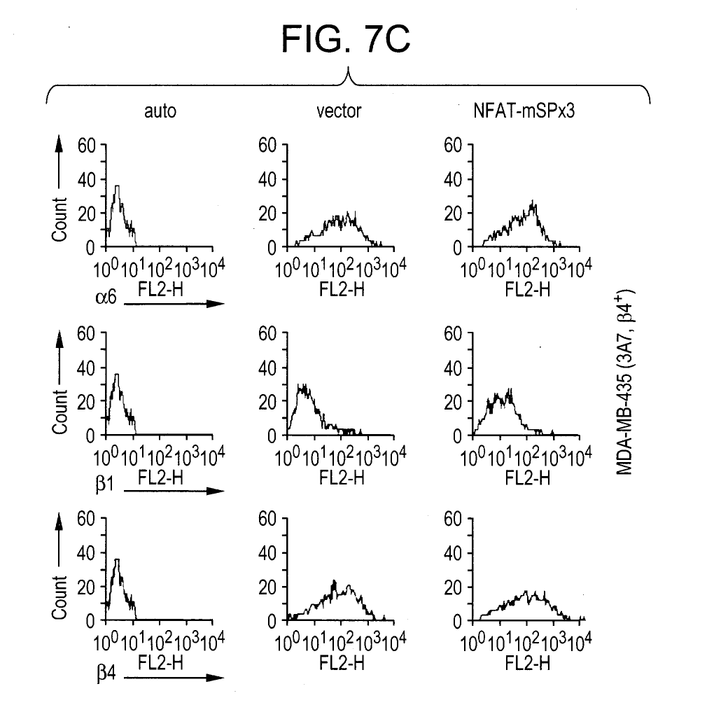

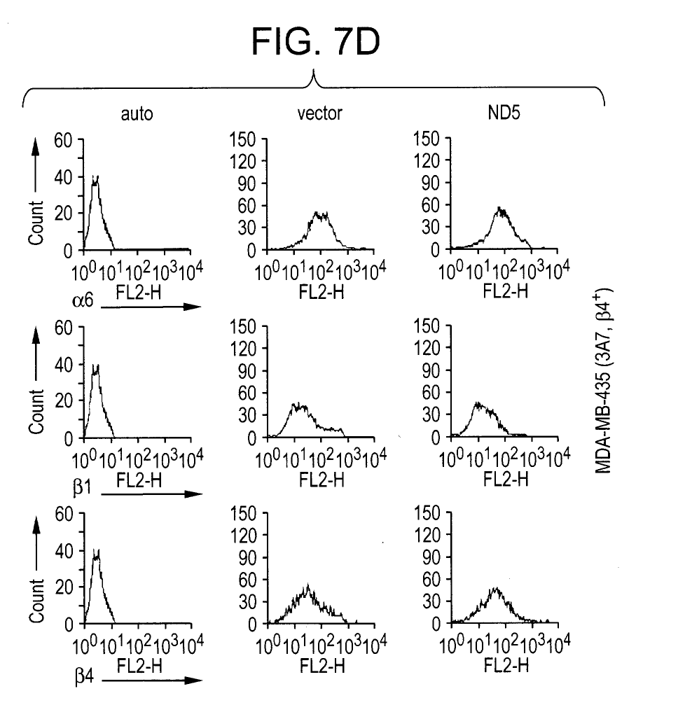

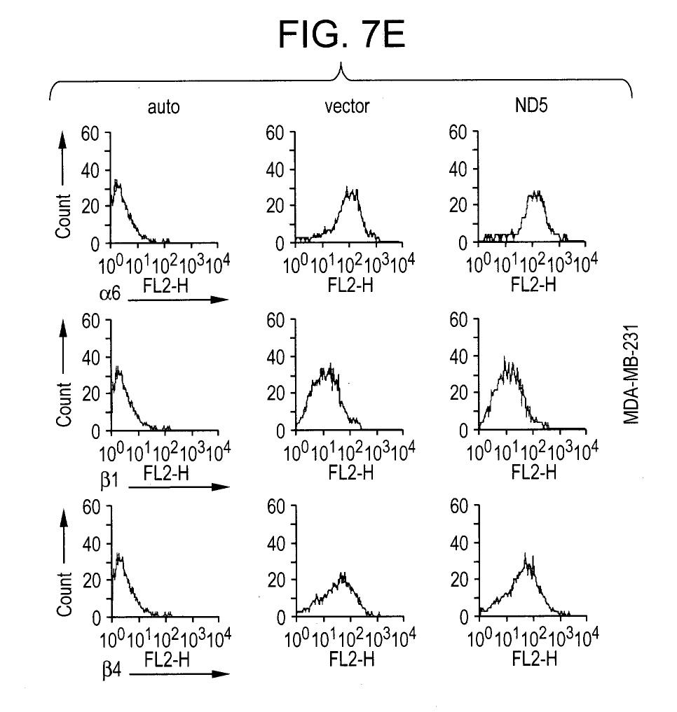

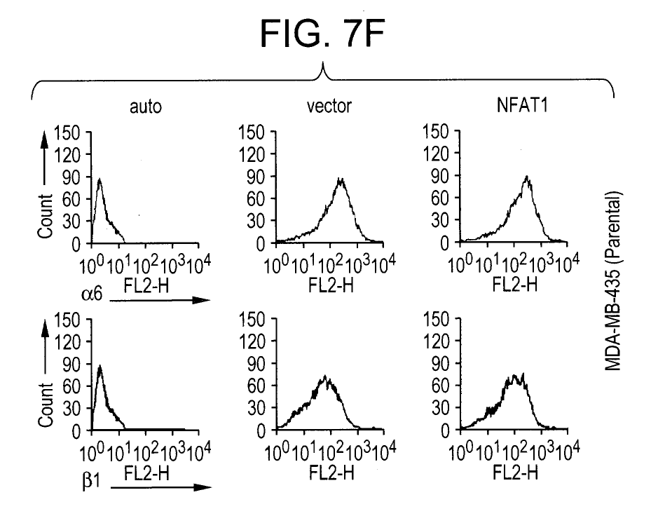

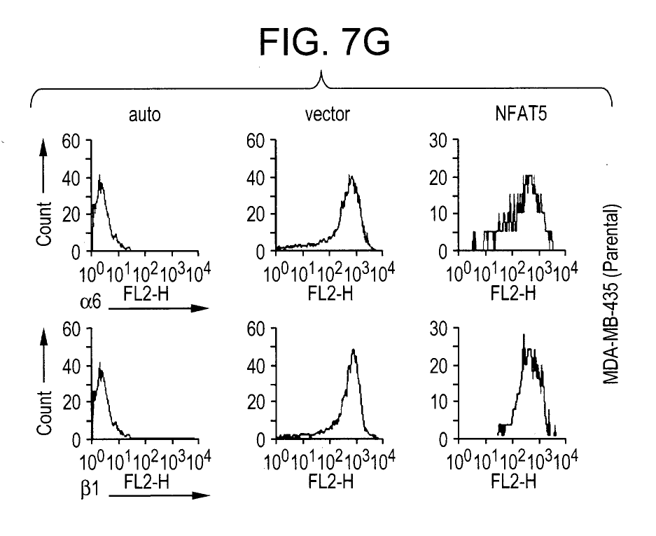

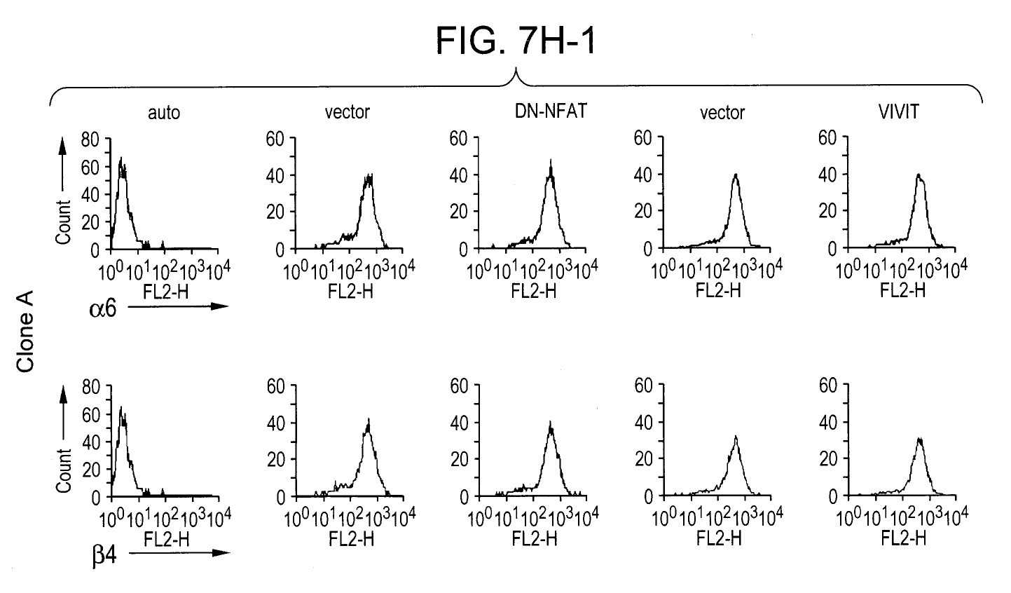

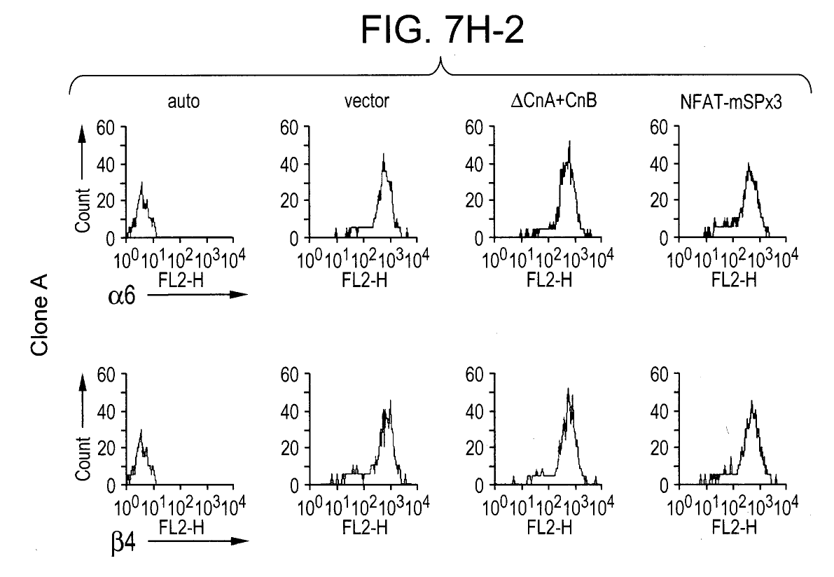

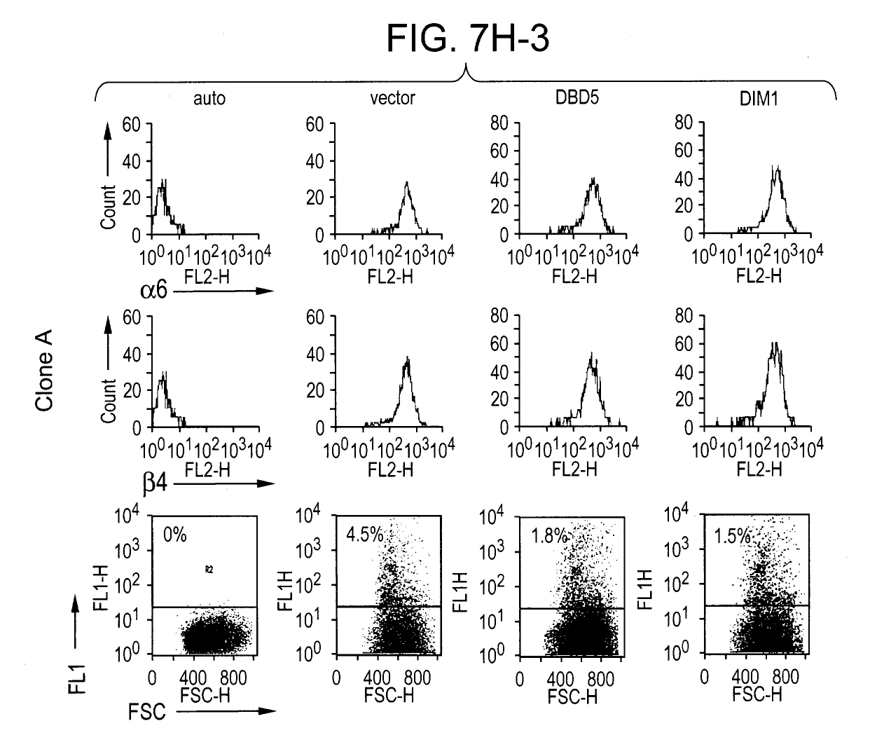

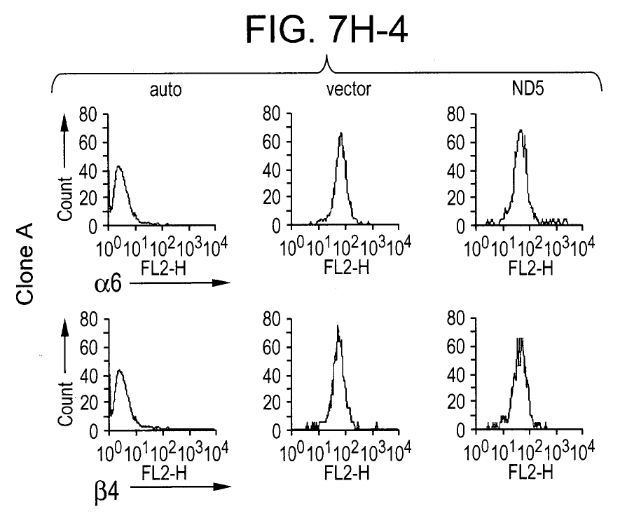

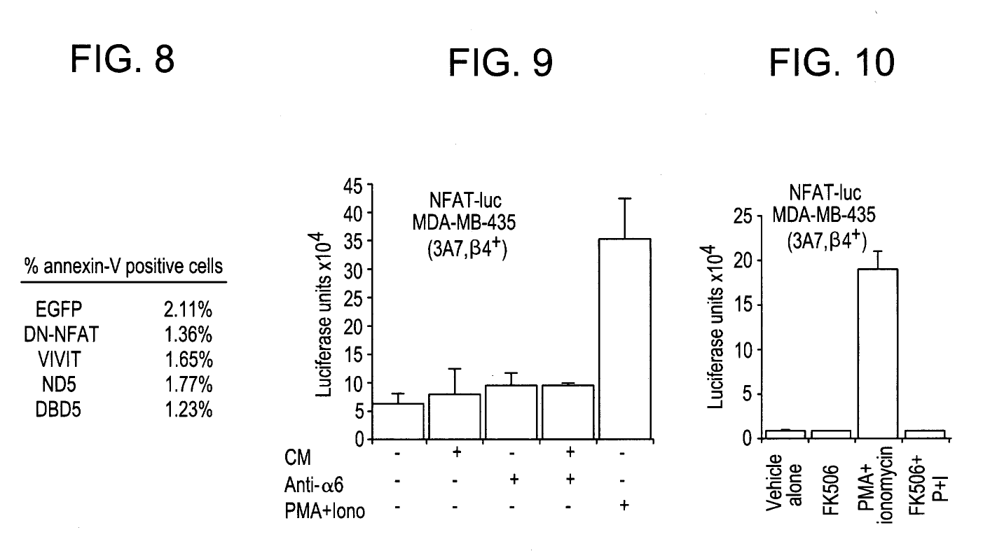

Collectively, these data indicate that conventional NFATs function in carcinoma migration and invasion. Expression of the NFAT mutants did not quantitatively alter the expression of the surface integrins, as judged by FACS analysis, and did not modify cell viability compared to cells transfected with vector alone (FIGS. 7A-7H, FIGS. 8, 9, and 10). Identical results were obtained in the Clone A colon cancer cell line, where the VIVIT peptide blocked invasion, and the activated NFAT and calcineurin mutants increased the basal level of carcinoma invasion (FIG. 2B). Importantly, the parental cell line (MDA-MB-435), which does not express α6β4, but does express NFAT-1, is also invasive, but to a lesser extent that the β4+-expressing transfectants (Shaw et al., Cell 91:949-960, 1997). In the parental cells, invasion is also blocked by the two dominant negative NFAT alleles, demonstrating that regardless of the expression of the β4 integrin, the invasive phenotype is NFAT-dependant. Therefore, expression of this integrin mediates amplification of PI 3-K (Shaw et al., Cell 91:949-960, 1997), Met (Trusolino et al., Cell 107:643-54, 2001), and NFAT pathways leading to invasion. These results showed that NFAT is both necessary and sufficient to induce carcinoma invasion. Significantly, this is the first demonstration that NFAT plays a role in mediating the invasive phenotype.

In α6β4-expressing cells, integrin engagement did not enhance the transcriptional activity of conventional NFAT, suggesting that the high basal NFAT activity observed in these cells is sufficient to promote invasion (see FIG. 7-10, inclusive). Neither cyclosporin A nor FK506 significantly inhibited α6β4-dependent carcinoma invasion in the Matrigel assay, although FK506 quantitatively blocked inducible NFAT activity when cells were stimulated with PMA (phorbol 12-myristate 13-acetate) and ionomycin. These antagonists did not block the high basal NFAT transcriptional activity that was observed in these cells (FIGS. 7A-7H, FIGS. 8, 9, 10). This correlates with the lack of inhibition in the invasion assay.

The Role of NFAT5 in Mediating Carcinoma Invasion

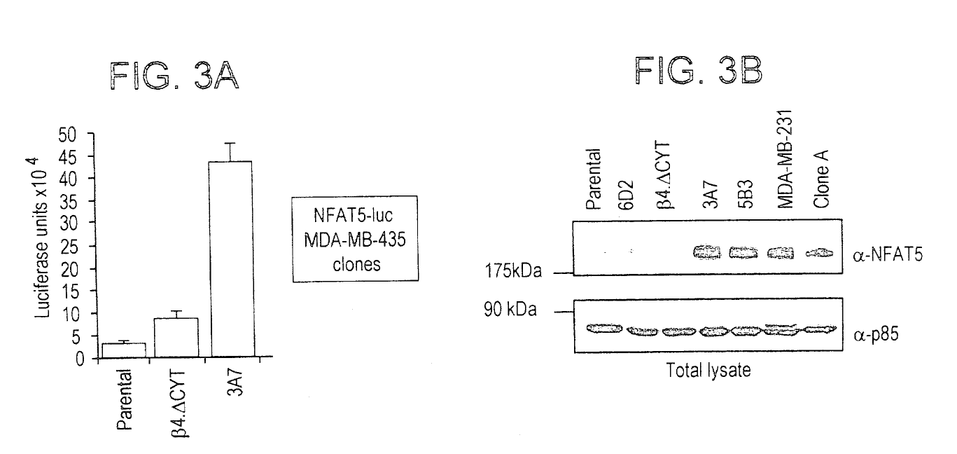

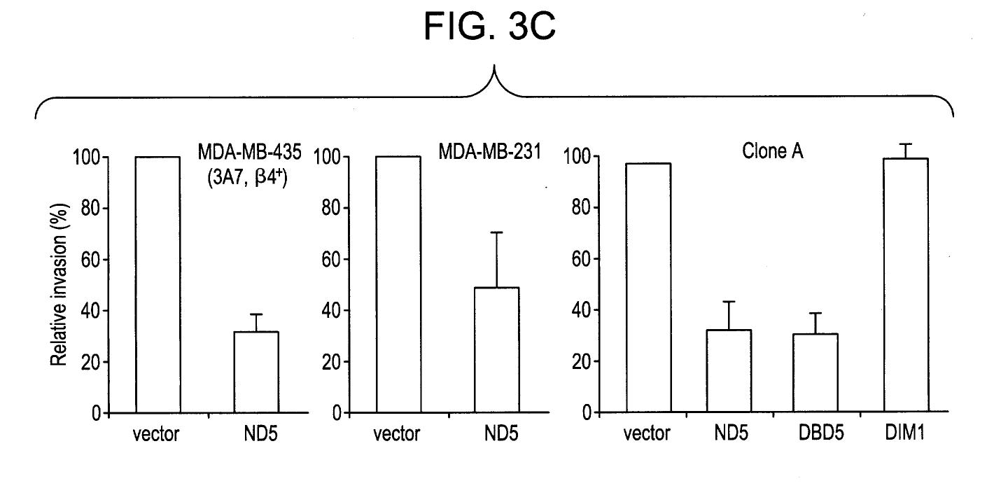

The activity of the novel and ubiquitously-expressed NFAT-5 family member is not regulated by calcineurin because it lacks the amino-terminal NFAT homology region, although it does contain an NFAT-related DNA-binding domain (Lopez-Rodriguez et al., Proc. Natl. Acad. Sci. USA 96:7214-9, 1999). As with conventional NFATs, the transcriptional activity of NFAT-5 was significantly increased in MDA-MB-435 cells expressing β4+ when compared to parental cells or cells expressing β4.ΔCYT (FIG. 3A).

We evaluated the expression of NFAT-5 in these cells using an NFAT-5-specific antibody in immunoblotting experiments. We found that the expression of NFAT-5 in β4+-expressing cells (β4+(3A7, 5B3), MDA-MB-231 and clone A) was significantly higher than in non-β4+-exprressing cells (FIG. 3B). Thus, as with conventional NFAT-1, increased NFAT-5 expression correlated with β4 expression.

We therefore evaluated the role of NFAT-5 in mediating carcinoma invasion in α6β4-expressing cells. Homodimerization of NFAT-5 is required to achieve full transcriptional activity (Lopez-Rodriguez et al., Immunity 15:47-58, 2001). A dominant negative NFAT-5 mutant (ND5), which blocks dimerization of endogenous NFAT-5, significantly inhibited invasion when transiently transfected into MDA-MB-435, MDA-MB-231, and Clone A cells (FIG. 3C). Moreover, a distinct NFAT-5 dominant negative mutant (DBD5) also inhibited invasion in clone A cells. An inactive DBD5 allele (DIM 1), which does not block dimerization, did not inhibit invasion (FIG. 3C). As with NFAT-1, the dominant negative NFAT-5 alleles were able to inhibit invasion in the parental MDA-MB-435 cell line.

NFAT5 Transcriptional Activity is Induced in an α6β4-Dependent Manner

Unlike calcineurin-sensitive NFAT, the transcriptional activity of NFAT-5 was inducible. The standard Matrigel chemoinvasion assay was carried out in the presence of conditioned media obtained from confluent NIH 3T3 fibroblasts. This media contains chemoattractants that carcinoma cells migrate towards in a chemotactic fashion. Both integrin ligation and conditioned media are required to mediate the full invasive phenotype.

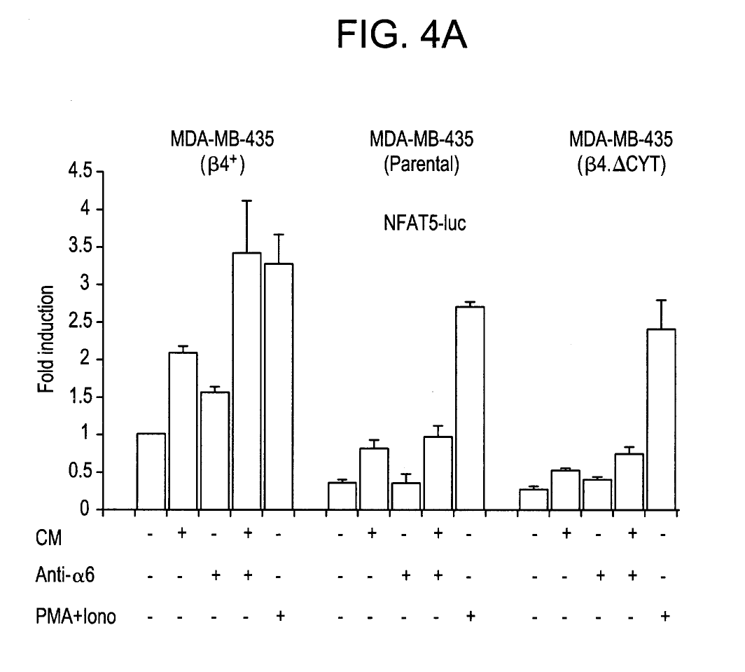

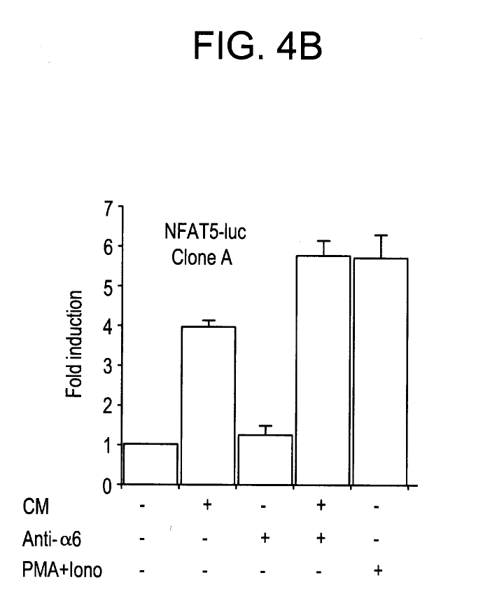

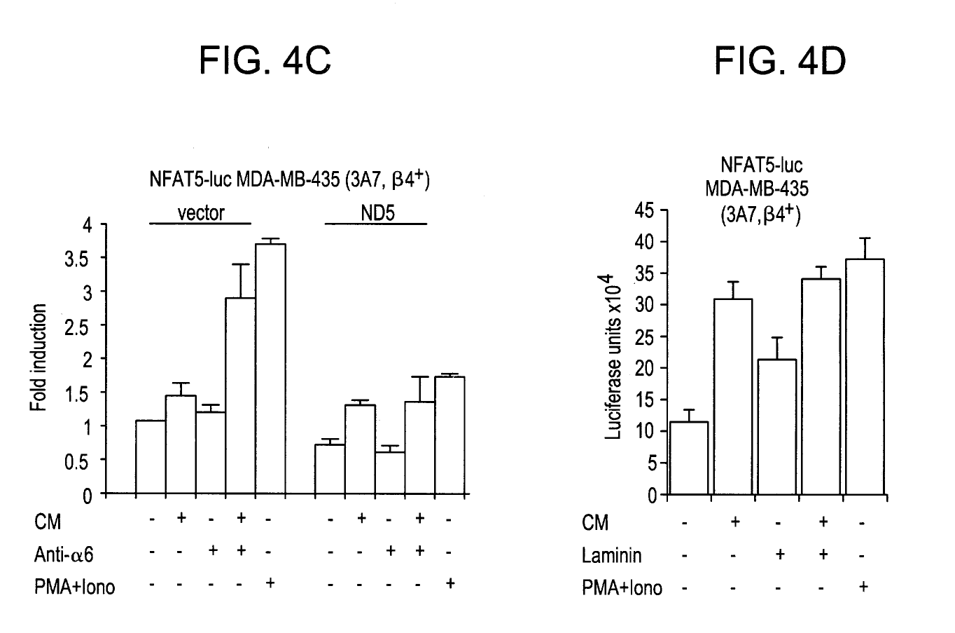

We found that clustering α6β4 with α6 monoclonal antibodies or adding conditioned media alone to MDA-MB-435 cells resulted in a small, but significant increase in NFAT-5 transcriptional activity (FIG. 4A). When integrin clustering was induced in the presence of conditioned media, there was an additive, 3-fold increase in NFAT-5 transcriptional activity that was comparable to activity levels achieved with the addition of PMA and ionomycin. Importantly, this co-stimulation of NFAT-5 activity required the presence of an intact β4cytoplasmic tail, as it was not observed in β4-negative or β4.ΔCYT-expressing cells. Co-stimulation of NFAT-5 transcriptional activity by α6β4 clustering and conditioned media was also observed in Clone A colon cancer cells (FIG. 4B).

Finally, co-stimulation of NFAT-5 transcriptional activity by α6β4 and conditioned media, as well as PMA/ionomycin treatment was inhibited when cells were co-transfected with the dominant negative NFAT-5 mutant (FIG. 4C) (Lopez-Rodriguez et al., Immunity 15:47-58, 2001). This inhibition correlated with the ability of this mutant to block carcinoma invasion in the Matrigel assay (FIG. 3C). Thus, NFAT-5 was not only transcriptionally active in these cells, but also participated in integrin-mediated invasion. NFAT-5 activity was also induced by plating cells on the α6β4 ligand, laminin-1. Maximal transcriptional activity was also observed when cells were plated on laminin-1 in the presence of conditioned media (FIG. 4D).

Overexpression of Wild-Type NFAT1 and NFAT5 in MDA-MB-435 Cells

The analysis of NFAT in the carcinoma cell lines tested here showed increased levels of NFAT-1 and NFAT-5 in β4⁺-expressing cells when compared to cells that do not express this integrin subunit (FIGS. 1D and 3B). Increased cell migration was intimately linked with carcinoma invasion. We tested whether overexpression of either wildtype NFAT-1 or NFAT-5 in the parental cell line MDA-MB-435, which does not express the β4 integrin, was sufficient to promote cellular migration and/or invasion. Chemotactic migration assays were carried in vector alone-, NFAT-1-, or NFAT-5-transiently transfected parental MDA-MB-435 cells in a transwell chamber in the absence of Matrigel and in the presence of conditioned media.

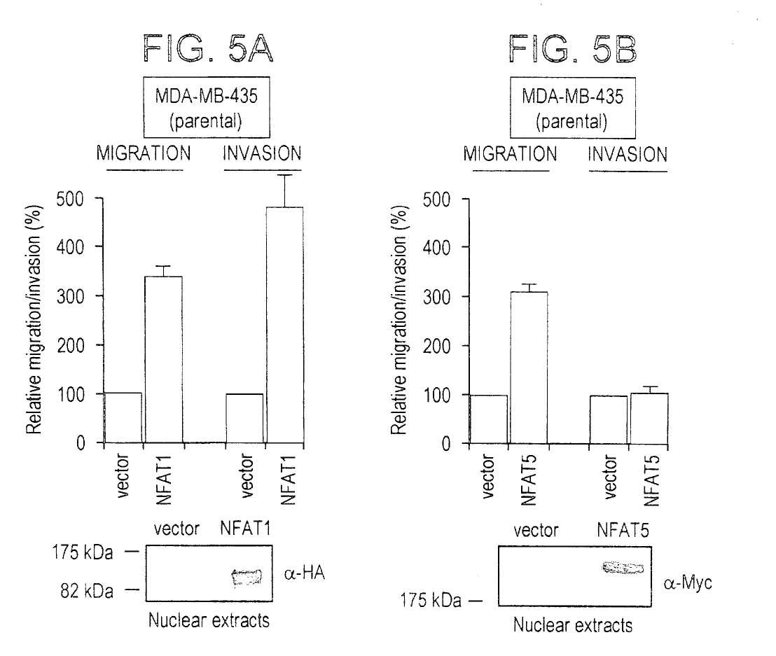

The results presented in FIG. 5 demonstrated that both wild-type NFAT-1 and wild-type NFAT-5 are sufficient to increase chemotactic migration of MDA-MB-435 cells. Strikingly, while expression of NFAT-1 also induced carcinoma invasion (FIG. 5A), this was not observed with the wild-type NFAT-5 allele (FIG. 5B). Immunoblot analysis of the nuclear extracts of the transfected cells revealed expression of NFAT 1 and NFAT-5 (FIGS. 5B and 5D). These results indicated that NFAT-1 and NFAT-5 likely induced a distinct subset of genes important for migration (NFAT-5) and invasion (NFAT-1). Since expression of the wild-type NFAT-1 allele was sufficient to promote invasion, likely targets of NFAT-1 transcriptional activity are the promoters of matrix metalloproteases, which are also critical for inducing the invasive phenotype (John & Tuszynski, Pathol. Oncol. Res. 7:14-23, 2001).

These results clearly indicated that NFAT expression in carcinoma cells induced an increased-migration phenotype. It is important to note that endogenous NFAT may promote carcinoma invasion in the absence of a functional α6β4 integrin, because wild-type NFAT alleles are able to promote invasion in parental MDA-MB-435 cells (FIG. 5), and dominant negative NFAT mutants block invasion in these cells (data not shown). This is reminiscent of PI 3-K-dependent invasion in these cells, which can occur in the absence of α6β4 signaling. NFAT1, NFAT5 and β4 integrin are expressed in human breast carcinomas We have shown that NFATs are functionally expressed in cell lines derived from human carcinomas. NFATs are not restricted to cells of the immune system (Graef et al., Curr. Opin. Genet. Dev. 11:505-12, 2001), and recent studies have revealed expression in other tissues including heart (de Ia Pompa et al., Nature 392:182-6, 1998), muscle (Boss et al., J. Biol. Chem. 273:19664-71, 1998), and brain (Plyte et al., J. Biol. Chem. 276:14350-8, 2001). At the time our experiments were conducted, no information existed regarding whether NFAT was expressed in epithelial tissues or tumors.

We evaluated the presence of NFAT in tissue samples derived from five breast cancer patients with grade III invasive ductal carcinoma. Sections of adjacent histologically normal breast tissue were also examined in two patients. Four of the five patients studied had lymph node metastases at the time of diagnosis.

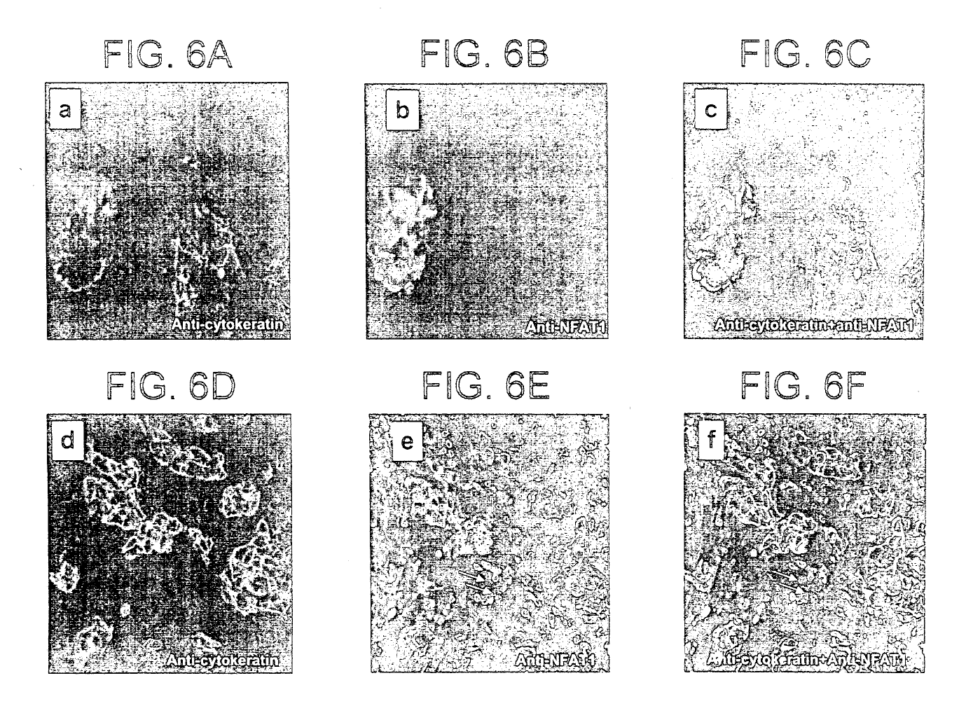

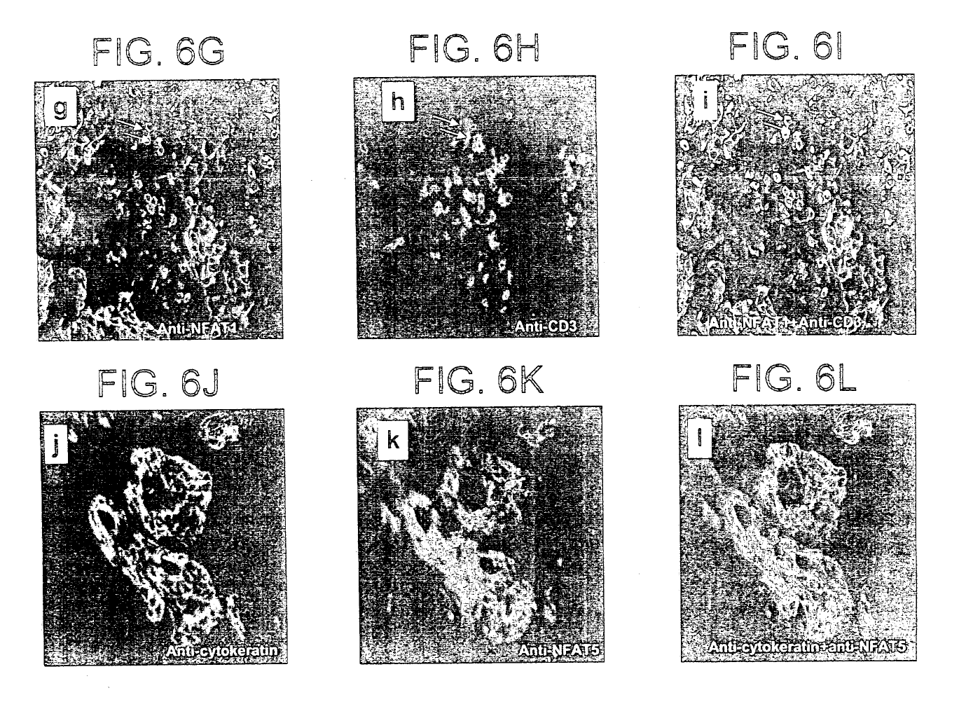

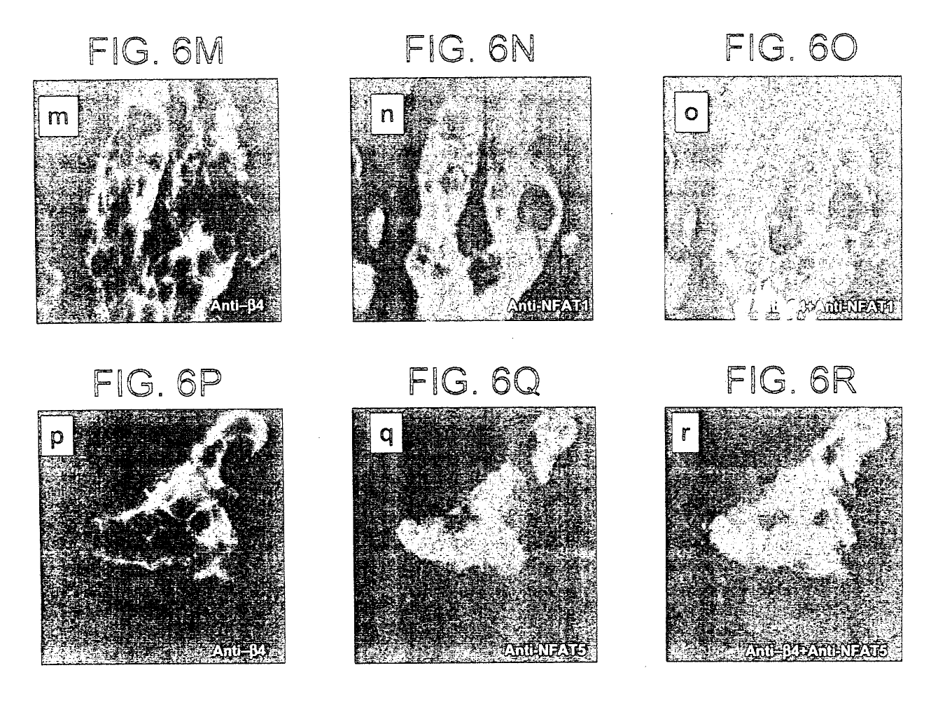

To evaluate NFAT expression, sections were labeled with an anti-cytokeratin antibody to identify the carcinoma, and with an anti-NFAT-1 antibody. The results in FIG. 6A-6C show two distinct breast carcinomas that scored positive for cytokeratin (FIG. 6A) and NFAT-1 expression (FIG. 6B). Only one of these carcinomas expressed NFAT-1 as shown in the overlay (FIG. 6C). Breast carcinoma from a distinct breast cancer patient (FIG. 6D-F) was also positive for NFAT-1 expression (FIG. 6E). Interestingly, in this section certain cells scored positive for NFAT-1 expression, but not for cytokeratin (indicated with arrows in FIGS. 6E and F). We hypothesize that these cells represent infiltrating lymphocytes, which are known to express NFAT-1. To test this hypothesis, a tissue section from this patient was labeled with anti-NFAT-1 (FIG. 6G) and an anti-CD3 antibody (FIG. 6H) to identify lymphocytes. Both the breast carcinoma and the surrounding cells scored positive for NFAT-1 (FIG. 6G), and cells present in the center of the section scored positive for both CD3 and NFAT-1 (FIGS. 6H and I) confirming their identity as infiltrating lymphocytes. In similar labeling experiments, we evaluated the presence of the recently identified NFAT-5 isoform. Like NFAT-1, tissue sections derived from grade III invasive ductal breast carcinoma showed a strong reactivity with an anti-NFAT-5 specific antibody (FIG. 6K), and this was found to co-localize with the carcinoma as shown in the anti-cytokeratin overlay (FIG. 6L).

The α6β4 integrin has been shown to be highly expressed in human breast carcinomas (Mecurio & Rabinovitz, Semin. Cancer Biol. 11:129-141, 2001). Conversely, β4 expression in normal tissue is primarily restricted to the surrounding myoepithelial cells (Koukoulis et al., Am. J. Pathol. 139:787-99, 1991; Tagliabue et al., Clin. Cancer Res. 4:407-10, 1998). We therefore evaluated the expression of the β4 integrin in these tissue sections. All sections scored positive for β4 expression (FIGS. 6M And 6P). Interestingly, we detected co-localization with both NFAT-1 (FIG. 60) and NFAT-5 (FIG. 6R). Therefore, human breast carcinomas that express the β4 integrin also express NFAT-1 and NFAT-5 (FIGS. 6A-6R). While we have detected expression of NFAT1 and NFAT5 in normal breast tissue, the restricted expression of the β4 integrin in breast carcinoma (Koukoulis et al., Am. J. Pathol. 139:787-99, 1991; Tagliabue et al., Clin. Cancer Res. 4:407-10, 1998) is consistent with the above data demonstrating that NFATs are targets of α6β4-dependent signaling leading to invasion. Whether altered expression or activation of NFATs occurs during the various stages of carcinoma progression remains to be determined.

These results provide evidence for a functional role of NFAT transcription factors in promoting invasion of carcinoma cells and highlight a role for the α6β4 integrin in the activation of conventional NFATs and NFAT5. This is the first demonstration that this family of transcription factors is expressed in human breast carcinomas and that they are functionally relevant for mediating invasion, one of the hallmarks of tumor metastasis. A number of important concepts have emerged from these studies; first, we have discovered that NFAT1 and NFAT5 are expressed in tissues derived from invasive human breast carcinomas. Second, we have found that the increased expression and/or activation of conventional NFAT isoforms and NFAT5 in breast and colon cancer cells is linked to the presence of a functional α6β4 integrin. In the case of NFAT1, the functional consequence is increased constitutive transcriptional activity, which is mediated, at least in part, by an increase in protein expression. The net result is an increase in carcinoma invasion that can be blocked with dominant negative NFAT alleles, but surprisingly, not with cyclosporin A or FK506. The most likely explanation for this observation is that in these carcinoma cells, conventional NFATs are constitutively active and this basal activity is not sensitive to antagonists that target the ability of immunophilins to bind to and regulate calcineurin.

Moreover, our results also show that unlike NFAT1, NFAT5 transcriptional activity is increased by engagement of the α6β4 integrin. Prior to our discover, only hyperosmotic stress, T cell activation through the T-cell receptor, and PMA/ionomycin treatment have been shown to activate NFAT5 (Lopez-Rodriguez et al., Immunity 15:47-58, 2001; Miyakawa, et al., Proc. Natl. Acad. Sci. USA 96:2538-42, 1999; Traim et al., J. Immunol. 165:4884-94, 2000). In kidney cells, the activation of NFAT5 by hyperosmotic stress is necessary to enable the cell to re-equilibrate its osmolality (Miyakawa et al., Am J. Physiol. 274:F753-61, 1998). In carcinoma cells, full NFAT5 activation is achieved by co-stimulation of the α6β4 integrin in the presence of chemoattractants present in conditioned medium derived from NIH 3T3 cells, and it is well established that it is also required for carcinoma invasion in the Matrigel assay. The identity of the ligands present in this medium which mediate this costimulation have yet to be identified, although potential candidates include chemokines such as CXCL12 and CCL21, which along with their receptors CXCR4 and CCR7 were recently shown to be highly expressed in breast cancer cells, malignant breast tumors and metastases (Muller et al., Nature 410:50-6, 2001).

These studies show for the first time the functional expression of NFAT in invasive human breast cancer and cells derived from breast carcinomas, and provide evidence for a previously uncharacterized signaling pathway leading to NFAT activation and carcinoma invasion. These unexpected results indicate that NFAT-dependent gene expression regulates the motility of immune cells, a phenotype that is critical for an efficient immune response. Given these results, the subset of target genes that are induced by NFAT1 and NFAT5 in invasive tumors and the importance of these genes in the progression of the disease can now be identified. The fact that NFAT1 is sufficient to promote both carcinoma migration and invasion, whereas NFAT5 is restricted to promoting carcinoma cell migration highlights the possibility of distinct genes induced by these transcription factors. Moreover, these results suggest novel avenues for drug discovery targeted towards blocking NFAT function.

Tissue Samples and Immunofluorescence

Tissue samples of primary tumors and adjacent breast tissue from untreated patients (n=5) with grade III invasive ductal breast carcinomas were obtained. Histopathological diagnosis was confirmed for each specimen. Institutional review board (IRB) approval for specimen collection was obtained. Tissues were collected fresh, thinly sliced, fixed in 4% paraformaldehyde for 4 hours at 4° C., incubated in 30% sucrose in phosphate buffered saline (PBS) overnight at 4° C. and embedded in TISSUETEK-OCT (optimal cutting temperature) compound (Sakura, Calif.) a water soluble glycol and resin compound. Immunostaining was performed on frozen sections. The anti-NFAT-1 antibody (clone 67.1, rabbit polyclonal) (Feske et al., J. Immunol. 165:297-305, 2000) was used at 1:200 dilution, the anti-NFAT-5 (rabbit polyclonal, AFFINITY BIOREAGENTS) at 1:200 dilution, the anti-cytokeratin (mouse monoclonal, SIGMA) at 1:100 dilution, the anti-CD3 (mouse monoclonal, UCHT1 clone, PHARMINGEN) at 1:200 dilution, and the anti-β4 (mouse monoclonal, ANCELL) at 1:200 dilution. Antibodies were diluted in 1% BSA (bovine serum albumin) and tris-buffered saline (50 mM Tris-Cl pH 7.4, 150 mM NaCl). Secondary fluorophore-coupled antibodies used were an anti-rabbit coupled to Cy-2 and an anti-mouse coupled to Cy-3. These were used at 1:200 dilution and 1:800 dilution according to manufacturer's instructions (JACKSON-IMMUNORESEARCH LABORATORIES). Before incubating the sections with specific antibodies, slides were blocked with a non-specific goat non-immune serum (Life Technologies).

Migration/Invasion Assays

Invasion assays were performed using standard methods (see Shaw et al., Cell 91:949-960, 1997) using Transwell chambers coated with Matrigel. Briefly, cells were co-transfected with the indicated NFAT constructs, 1 μg EGFP-N1 (Clontech) and 1 μg pCS2-(n)β-Gal, a •gal expression vector (Dave Turner, University of Michigan) using the Lipofectamine procedure (Life Technologies), and after twenty-four hours, the cells were resuspended in serum-free medium containing 0.1% bovine serum albumin (BSA) and added to each well. After 6 hours cells that had migrated to the lower surface of the filters were fixed in 4% paraformaldehyde and stained with PBS containing 1 mg/ml BLUO-GAL (BioVectra, P.E.I., Canada) (5-BROMO-3-INDOLYL-BETA-D-GALACTOPYRANOSIDE). Cells that stained positive for β-galactosidase expression were counted, and the mean of triplicate assays for each experimental condition was used as % relative invasion. The co-transfection of EGFP-N1 plasmid was used to analyze by FACS the expression of the α6, β1 and β4 integrins of transfected cells. For migration assays, the same procedure was performed except that Transwell chambers were not coated with Matrigel.

Electrophoretic Mobility Shift Assay (EMSA)

Growing cells were washed in PBS and resuspended in hypotonic buffer (10 mM Hepes pH 7.9, 10 mM KCl, 0.1 mM EDTA, 0.1 mM EGTA, 1 mM DTT, 1 mM PMSF) with 0.1% NP-40. After centrifugation, nuclear pellets were washed twice in hypotonic buffer and lysed in high salt buffer (20 mM Hepes pH 7.9, 400 mM NaCl, 1 mM EDTA, 1 mM EGTA, 1 mM DTT, 1 mM PMSF). The same quantity of protein from the resulting nuclear extracts for each clone was used in an EMSA using NFAT or AP-1 nucleotide probes. For supershift experiments nuclear extracts were preincubated with non-immune serum or specific antibodies against NFAT-1 (anti-NFAT-1, clone 67-1) prior to addition of the labeled probe, and separated on a 6% polyacrylamide gel.

Cell Transfections

Cells were transiently co-transfected with an NFAT luciferase reporter construct (NFAT-luc, 10 μg) and 1 μg pCS2-(n)β-gal using the Lipofectamine procedure (Life Technologies). Twenty-four hours after the transfection, luciferase and beta-galactosidase assays were performed using standard assays on total cell lysates and measured on a luminometer. For each clone the luciferase activity was normalized to the beta-galactosidase activity.

Identification of Compounds that Modulate NFAT Biological Activity

Methods of observing changes in NFAT interactions and NFAT biological activity are exploited in high throughput assays for the purpose of identifying compounds that modulate NFAT protein-protein or protein-nucleic acid interactions. Compounds that inhibit NFAT binding to transcription factor partners, to NFAT consensus DNA sequences, or that inhibit NFAT biological activity (e.g., NFAT's activity as a transcriptional activator or repressor), may be identified by such assays. In addition, compounds that modulate the expression of a NFAT polypeptide or nucleic acid molecule whose expression is altered in a patient having a neoplasia may be identified.

Any number of methods are available for carrying out screening assays to identify new candidate compounds that decrease the expression of an NFAT nucleic acid molecule. In one working example, candidate compounds are added at varying concentrations to the culture medium of cultured cells expressing one of the nucleic acid sequences of the invention. Gene expression is then measured, for example, by microarray analysis, Northern blot analysis (Ausubel et al., supra), or RT-PCR, using any appropriate fragment prepared from the nucleic acid molecule as a hybridization probe. The level of gene expression in the presence of the candidate compound is compared to the level measured in a control culture medium lacking the candidate molecule. A compound which promotes an increase in the expression of a NFAT gene, or a functional equivalent thereof, is considered useful in the invention; such a molecule may be used, for example, as a therapeutic to treat a neoplasia in a human patient.

In another working example, the effect of candidate compounds may be measured at the level of polypeptide production using the same general approach and standard immunological techniques, such as Western blotting or immunoprecipitation with an antibody specific for a polypeptide encoded by an NFAT gene. For example, immunoassays may be used to detect or monitor the expression of at least one of the polypeptides of the invention in an organism. Polyclonal or monoclonal antibodies (produced as described above) that are capable of binding to such a polypeptide may be used in any standard immunoassay format (e.g., ELISA, Western blot, or RIA assay) to measure the level of the polypeptide. In some embodiments, a compound that promotes an increase in the expression or biological activity of the polypeptide is considered particularly useful. Again, such a molecule may be used, for example, as a therapeutic to delay, ameliorate, or treat a neoplasia in a human patient.

In yet another working example, candidate compounds may be screened for those that specifically bind to a polypeptide encoded by an NFAT gene. The efficacy of such a candidate compound is dependent upon its ability to interact with such a polypeptide or a functional equivalent thereof. Such an interaction can be readily assayed using any number of standard binding techniques and functional assays (e.g., those described in Ausubel et al., supra). In one embodiment, a candidate compound may be tested in vitro for its ability to specifically bind a polypeptide of the invention. In another embodiment, a candidate compound is tested for its ability to inhibit the biological activity of a polypeptide described herein, such as a NFAT polypeptide. The biological activity of an NFAT polypeptide may be assayed using any standard method, for example, a matrigel cell invasion or cell migration assay.

In another working example, a nucleic acid described herein (e.g., an NFAT nucleic acid) is expressed as a transcriptional or translational fusion with a detectable reporter, and expressed in an isolated cell (e.g., mammalian or insect cell) under the control of a heterologous promoter, such as an inducible promoter. The cell expressing the fusion protein is then contacted with a candidate compound, and the expression of the detectable reporter in that cell is compared to the expression of the detectable reporter in an untreated control cell. A candidate compound that alters the expression of the detectable reporter is a compound that is useful for the treatment of a neoplasia. Preferably, the compound decreases the expression of the reporter.

In one particular working example, a candidate compound that binds to a polypeptide encoded by an NFAT gene may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the NFATpolypeptide is identified on the basis of its ability to bind to the polypeptide and be immobilized on the column. To isolate the compound, the column is washed to remove non-specifically bound molecules, and the compound of interest is then released from the column and collected. Similar methods may be used to isolate a compound bound to a polypeptide microarray. Compounds isolated by this method (or any other appropriate method) may, if desired, be further purified (e.g., by high performance liquid chromatography). In addition, these candidate compounds may be tested for their ability to increase the activity of an NFAT polypeptide (e.g., as described herein). Compounds isolated by this approach may also be used, for example, as therapeutics to treat a neoplasia in a human patient. Compounds that are identified as binding to a polypeptide of the invention with an affinity constant less than or equal to 10 mM are considered particularly useful in the invention. Alternatively, any in vivo protein interaction detection system, for example, any two-hybrid assay may be utilized.

Potential antagonists include organic molecules, peptides, peptide mimetics, polypeptides, nucleic acids, and antibodies that bind to a nucleic acid sequence or polypeptide of the invention (e.g., an NFAT polypeptide or nucleic acid molecule).

Each of the DNA sequences listed herein may also be used in the discovery and development of a therapeutic compound for the treatment of neoplasia. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques (Ausubel et al., supra).

Optionally, compounds identified in any of the above-described assays may be confirmed as useful in an assay for compounds that modulate the propensity of a neoplasia to metastasize.

Small molecules of the invention preferably have a molecular weight below 2,000 daltons, more preferably between 300 and 1,000 daltons, and most preferably between 400 and 700 daltons. It is preferred that these small molecules are organic molecules.

Test Extracts and Compounds

In general, compounds that modulate NFAT expression, biological activity, or NFAT-dependent signaling are identified from large libraries of both natural products, synthetic (or semi-synthetic) extracts or chemical libraries, according to methods known in the art. Preferably, these compounds decrease NFAT expression or biological activity.

Those skilled in the art will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modifications of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from, for example, Brandon Associates (Merrimack, N.H.), Aldrich Chemical (Milwaukee, Wis.), and Talon Cheminformatics (Acton, Ont.)

Alternatively, libraries of natural compounds in the form of bacterial, fungal, plant, and animal extracts are commercially available from a number of sources, including, but not limited to, Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art (e.g., by combinatorial chemistry methods or standard extraction and fractionation methods). Furthermore, if desired, any library or compound may be readily modified using standard chemical, physical, or biochemical methods.

NFAT Production

In general, polypeptides of the invention, such as NFAT may be produced by transformation of a suitable host cell, for example, a eukaryotic cell, with all or part of a polypeptide-encoding nucleic acid molecule, or a fragment thereof in a suitable expression vehicle.

Those skilled in the field of molecular biology will understand that any of a wide variety of expression systems may be used to provide the recombinant protein. Eukaryotic NFAT peptide expression systems may be generated in which an NFAT peptide gene sequence is introduced into a plasmid or other vector, which is then used to transform living cells. Constructs in which the NFAT peptide cDNA contains the entire open reading frame inserted in the correct orientation into an expression plasmid may be used for protein expression. Eukaryotic expression systems allow for the expression and recovery of NFAT peptide fusion proteins in which the NFAT peptide is covalently linked to a tag molecule that facilitates identification and/or purification. An enzymatic or chemical cleavage site can be engineered between the NFAT peptide and the tag molecule so that the tag can be removed following purification.

Typical expression vectors contain promoters that direct the synthesis of large amounts of mRNA corresponding to the inserted NFAT nucleic acid in the plasmid-bearing cells. They may also include an origin of replication sequence allowing for their autonomous replication within the host organism, sequences that encode genetic traits that allow vector-containing cells to be selected for in the presence of otherwise sequences that increase the efficiency with which the synthesized mRNA is translated. Stable long-term vectors may be maintained as freely replicating entities by using regulatory elements of, for example, viruses (e.g., the OriP sequences from the Epstein Barr Virus genome). Cell lines may also be produced that have integrated the vector into the genomic DNA, and in this manner the gene product is produced on a continuous basis.

The precise host cell used is not critical to the invention. A polypeptide of the invention may be produced in any eukaryotic host (e.g., Saccharomyces cerevisiae, insect cells, such as Sf21 cells, or mammalian cells, such as NIH 3T3, HeLa, COS cells, or fibroblasts). Such cells are available from a wide range of sources (e.g., the American Type Culture Collection, Rockland, Md.; also, see, e.g., Ausubel et al., Current Protocols in Molecular Biology, Wiley Interscience, New York, 2001). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

Native NFAT can be isolated from human cells that produce it naturally, or from transgenic eukaryotic cells that have been engineered to express a recombinant NFAT gene. Methods for natural or recombinant production of NFAT are generally described in U.S. Pat. Nos. 4,758,510, 4,124,702, 5,827,694, 4,680,261, 5,795,779, 5,376,567, and 4,130,641.

Once the appropriate expression vectors are constructed, they are introduced into an appropriate host cell by transformation techniques, such as, but not limited to, calcium phosphate transfection, DEAE-dextran transfection, electroporation, microinjection, protoplast fusion, or liposome-mediated transfection.

Once the recombinant polypeptide of the invention is expressed, it is isolated, e.g., using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra). The recombinant protein can be purified by any appropriate techniques, including, for example, high performance liquid chromatography chromatography or other chromatographies (see, e.g., Fisher, Laboratory Techniques In Biochemistry And Molecular Biology, eds., Work and Burdon, Elsevier, 1980).

Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.).

These general techniques of polypeptide expression and purification can also be used to produce and isolate useful peptide fragments or analogs (described herein).

Target Gene Identification

Methods useful for the identification of target genes are well known to the skilled artisan and include, but are not limited to, serial analysis of gene expression (SAGE), differential display, DNA or RNA oligonucleotide microarray analysis, yeast 2-hybrid analysis (or variations thereof), chromatin immunoprecipitation (ChIP analysis), or Rnase protection assays. Many of the above-mentioned techniques are standard in the art (see for example, in Expression Genetics: Accelerated and high-throughput methods, Eds. McClelland, M. and Pardee, A. 1999, BioTechniques Press; Takahashi et al., Science's STKE (stke@sciencemag.org) Oct. 31, 2000; Carter et al., Proc. Natl. Acad. Sci. 96:13118-23, 1999; Velculescu et al., Science 270:484-487, 1995). Typically, microarray analysis is accomplished by hybridizing mRNAs (or cDNAs) extracted from the cells of interest onto fixed arrays of nucleotides. Control and NFAT over-expressing cells can be analysed, and differences in gene expression determined. Optionally, a subtractive hybridization step may be carried out or mRNAs may be extracted following polysome fractionation, allowing highly translated RNA species to be isolated.

Therapeutic Uses

The present invention features methods for treating neoplasia or the progression of neoplasia by administering NFAT nucleic acid molecules (e.g., double stranded NFAT RNAs or dominant negative NFAT alleles), NFAT polypeptides (e.g., a polypeptide having a dominant negative mutation), or compounds that decrease the expression or biological activity of an NFAT nucleic acid molecule or polypeptide. Compounds of the present invention may be administered by any appropriate route for the treatment or prevention of neoplasia. These may be administered to humans, domestic pets, livestock, or other animals with a pharmaceutically acceptable diluent, carrier, or excipient, in unit dosage form. Administration may be parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in Remington: The Science and Practice of Pharmacy (20th ed., ed. A. R. Gennaro, 2000, Lippincott Williams & Wilkins). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycholate and deoxycholate, or may be oily solutions for administration in the form of nasal drops, or as a gel. The concentration of the compound in the formulation will vary depending upon a number of factors, including the dosage of the drug to be administered, and the route of administration.

The compound may be optionally administered as a pharmaceutically acceptable salt, such as a non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like.

Administration of compounds in controlled release formulations is useful where the compound of formula I has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release in which the rate of release outweighs the rate of metabolism of the therapeutic compound. For example, controlled release can be obtained by the appropriate selection of formulation parameters and ingredients, including, e.g., appropriate controlled release compositions and coatings. Examples include single or multiple unit tablet or capsule compositions, oil solutions, suspensions, emulsions, microcapsules, microspheres, nanoparticles, patches, and liposomes.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

Microarrays

The nucleic acid molecules or polypeptides of the invention are useful as hybridizable array elements in a microarray. The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28:e3.i-e3.vii, 2000), MacBeath et al., (Science 289:1760-1763, 2000), Zhu et al. (Nature Genet. 26:283-289), and in U.S. Pat. No. 6,436,665, hereby incorporated by reference.

Nucleic Acid Microarrays

To produce a nucleic acid microarray oligonucleotides may be synthesized or bound to the surface of a substrate using a chemical coupling procedure and an ink jet application apparatus, as described in PCT application WO95/251116 (Baldeschweiler et al.), incorporated herein by reference. Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient, preferably as a bodily fluid (such as blood, cerebrospinal fluid, phlegm, saliva, or urine) or tissue sample (e.g. a tissue sample obtained by biopsy). For some applications, cultured cells (e.g., lymphocytes) or other tissue preparations may be used. The mRNA is isolated according to standard methods, and cDNA is produced and used as a template to make complementary RNA suitable for hybridization. Such methods are described herein. The RNA is amplified in the presence of fluorescent nucleotides, and the labeled probes are then incubated with the microarray to allow the probe sequence to hybridize to complementary oligonucleotides bound to the microarray.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art.

A detection system may be used to measure the absence, presence, and amount of hybridization for all of the distinct sequences simultaneously (e.g., Heller et al., Proc. Natl. Acad. Sci. 94:2150-2155, 1997). Preferably, a scanner is used to determine the levels and patterns of fluorescence.

Protein Microarrays

Families of proteins, such as those described herein, may also be analyzed using protein microarrays. Such arrays are useful in high-throughput low-cost screens to identify peptide or candidate compounds that bind a polypeptide of the invention, or fragment thereof. Typically, protein microarrays feature a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., polypeptides encoded by a nucleic acid molecule listed in Table 2 or Table 4 or antibodies against such polypeptides) are spotted on a substrate using any convenient method known to the skilled artisan (e.g., by hand or by inkjet printer). Preferably, such methods retain the biological activity or function of the protein bound to the substrate (Ge et al., supra; Zhu et al., supra).

The protein microarray is hybridized with a detectable probe. Such probes can be polypeptide, nucleic acid, or small molecules. For some applications, polypeptide and nucleic acid probes are derived from a biological sample taken from a patient, such as a bodily fluid (such as blood, urine, saliva, or phlegm); a homogenized tissue sample (e.g. a tissue sample obtained by biopsy); or cultured cells (e.g., lymphocytes). Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

Gene Therapy

Gene therapy is another therapeutic approach in which nucleic acid encoding dominant negative or inactivating NFAT mutants are introduced into cells. The transgene is delivered to cells in a form in which it can be taken up and expressed in an effective amount to inhibit neoplasia progression.

Transducing retroviral, adenoviral, or human immunodeficiency viral (HIV) vectors are used for somatic cell gene therapy because of their high efficiency of infection and stable integration and expression (see, for example, Cayouette et al., Hum. Gene Ther., 8:423-430, 1997; Kido et al., Curr. Eye Res. 15:833-844, 1996; Bloomer et al., J. Virol. 71:6641-6649, 1997; Naldini et al., Science 272:263-267, 1996; Miyoshi et al., Proc. Natl. Acad. Sci. USA, 94:10319-10323, 1997). For example, NFAT nucleic acid, or portions thereof, can be cloned into a retroviral vector and driven from its endogenous promoter, from the retroviral long terminal repeat, or from a promoter specific for the target cell type of interest (such as epithelial carcinoma cells). Other viral vectors that can be used include, but are not limited to, adenovirus, adeno-associated virus, vaccinia virus, bovine papilloma virus, vesicular stomatitus virus, or a herpes virus such as Epstein-Barr Virus.

Gene transfer can be achieved using non-viral means requiring infection in vitro. This would include calcium phosphate, DEAE-dextran, electroporation, and protoplast fusion. Liposomes may also be potentially beneficial for delivery of DNA into a cell. Although these methods are available, many of these are of lower efficiency.

Combination Therapies

NFAT nucleic acids of polypeptides may be administered in combination with any other standard neoplasia therapy; such methods are known to the skilled artisan (e.g., Wadler et al., Cancer Res. 50:3473-86, 1990), and include, but are not limited to, chemotherapy, hormone therapy, immunotherapy, radiotherapy, and any other therapeutic method used for the treatment of neoplasia.

Patient Monitoring

The disease state or treatment of a patient having neoplasia can be monitored using the methods and compositions of the invention. In one embodiment, a microarray is used to assay the expression profile of an NFAT nucleic acid molecuel. Such monitoring may be useful, for example, in assessing the efficacy of a particular drug in a patient. Therapeutics that decrease the expression of at least one NFAT or integrin nucleic acid molecule or polypeptide are taken as particularly useful in the invention.

While methods of neoplasia treatment vary depending on the type of neoplasia, the stage of neoplasia, and the patient's age, health, and physical condition, more aggressive treatment regimens will be used in patients having a poor prognosis (e.g., patients having a metastatic neoplasia or a neoplasia with a high metastatic potential). As described above, the methods of the invention are useful in determining the prognosis of a patient having neoplasia, such as a neoplasia with increased metastatic potential. In such patients aggressive therapies may be used. These include therapies having increased toxicity and those having an increased risk of adverse side-effects. Aggressive therapies are employed earlier and at higher doses in patients having a poor prognosis.

Other Embodiments

From the foregoing description, it is apparent that variations and modifications may be made to the invention described herein to adopt it to various usages and conditions. Such embodiments are also within the scope of the following claims. All publications mentioned in this specification are herein incorporated by reference to the same extent as if each independent publication or patent application was specifically and individually indicated to be incorporated by reference.

1. A method of assessing a patient as having, or having a propensity to develop, a neoplasia, said method comprising determining the level of expression of an NFAT nucleic acid molecule or polypeptide in a patient sample, wherein an increased level of expression relative to the level of expression in a control sample, indicates that said patient has or has a propensity to develop a neoplasia.

2. The method of claim 1, wherein said patient sample is a blood or tissue sample.

3. The method of claim 1, wherein said method further comprises the step of determining the level of expression of a β4 integrin subunit nucleic acid molecule or polypeptide relative to the level in a control sample.

4. The method of claim 3, wherein said β4 integrin subunit is α6β4.

5. The method of claim 1, wherein said method comprises determining the level of expression of said NFAT nucleic acid molecule.

6. The method of claim 1, wherein said method comprises determining the level of expression of said NFAT polypeptide.

7. The method of claim 6, wherein said level of expression is determined in an immunological assay.

8. The method of claim 1, wherein said method is used to diagnose a patient as having neoplasia.

9. The method of claim 1, wherein said method is used to determine the treatment regimen for a patient having neoplasia.

10. The method of claim 1, wherein said method is used to monitor the condition of a patient being treated for neoplasia.

11. The method of claim 1, wherein said method is used to determine the prognosis of a patient having neoplasia.

12. The method of claim 11, wherein a poor prognosis determines an aggressive treatment regimen for said patient.

13. A method of diagnosing a patient as having a neoplasia with a propensity to metastasize, said method comprising determining the level of expression of an NFAT nucleic acid molecule or polypeptide in a patient sample, wherein an altered level of expression relative to the level of expression in a control sample, indicates that said patient has a neoplasia with a propensity to metastasize.

14. The method of claim 13, wherein said patient sample is a blood or tissue sample.

15. The method of claim 13, wherein said method further comprises the step of determining the level of expression of a β4 integrin subunit relative to the level in a control sample.

16. The method of claim 14, wherein said β4 integrin subunit is α6β4.

17. The method of claim 13, wherein said NFAT nucleic acid molecule or polypeptide is NFAT-1, NFAT-4, or NFAT-5.

18. The method of claim 13, wherein said method comprises determining the level of an NFAT nucleic acid molecule.

19. The method of claim 13, wherein said method comprises determining the level of an NFAT polypeptide.

20. The method of claim 13, wherein said level of expression is determined in an immunological assay.

21. The method of claim 13, wherein said alteration is an increase in the level of expression of an NFAT nucleic acid molecule.

22. The method of claim 21, wherein said increase determines an aggressive treatment regimen for said patient.

23. A diagnostic kit for the diagnosis of a neoplasia in a patient comprising a nucleic acid or amino acid sequence, or fragment thereof, selected from the group consisting of an NFAT-1, NFAT-4, or NFAT-5 nucleic acid, or fragment thereof, and any combination thereof.

24. The diagnostic kit of claim 23, further comprising a β4 integrin subunit.

25. The diagnostic kit of claim 23, wherein said β4 integrin subunit is α6β4.
